(19) United States
(12) Patent Application Publication
(10) Pub. No.: US 2021/0120808 A1
(43) Pub. Date: Apr. 29, 2021

(54) Methods and Systems for Synergistic Continuity Approaches to Treatment and Preservation of Biological Cells

(71) Applicant: Membrane Protective Technologies, Inc., Fort Collins, CO (US)

(72) Inventors: LIsa A. Herickhoff, Fort Collins, CO (US); Myles Shepherd, Nunn, CO (US); Christopher Bennett, Loveland, CO (US)

(21) Appl. No.: 16/766,254

(22) PCT Filed: Nov. 21, 2018
(86) PCT No.: PCT/US2018/062269

§ 371 Date: May 21, 2020

Related U.S. Application Data
(60) Provisional application No. 62/589,422, filed on Nov. 21, 2017.
(60) Provisional application No. 62/594,394, filed on Dec. 4, 2017.

(51) Int. Cl. A01N 1/02

(52) U.S. Cl. CPC A01N 1/0226; A01N 1/0215; A01N 1/0284

(57) ABSTRACT

Embodiments of the present invention may provide synergistic continuity approaches to treatment of biological cells which may mitigate damage thereto including but not limited to preserving a collection of biological cells (6) harvested from a in vivo source (7) perhaps in a holding media (10) which may be adapted from an anticipated cell damage limiting regimen (8) and even a predetermined use (9). Some embodiments of the present invention provide a uniform environment (15) around biological cells.

### PRORITY CLAIM

This application is an international PCT application claiming priority to and the benefit of U.S. Provisional Application No. 62/589,422 filed Nov. 21, 2017 and U.S. Provisional Application No. 62/594,394 filed Dec. 4, 2017, each hereby incorporated by reference herein.

### TECHNICAL FIELD

The present invention may relate to synergistic continuity approaches to treatment of biological cells which may mitigate damages associated with environmental exposure, movement, and storage that can occur right after harvesting cells and during transportation to a processing facility and perhaps through the steps of preservation and later use or analysis. Treatment of the cells may be provided perhaps by the addition of natural solutions such as with various extracts or the like. Biological cells may be protected with highly uniform environments for each cell.

### BACKGROUND OF THE INVENTION

In vitro processing of biological cells or tissues may include removal of cells from their native, perhaps in vivo, environment, transporting such cells, perhaps preserved in some manner, then utilized and/or analyzed at some future time. Specifically, there may be five steps in the processing of biological cells. A first step may be a harvesting step, then a transportation step, then a cooling or even a cryopreservation step, then a thawing or even a warming step, and finally using the cells such as implantation, fertilization, in vivo use, in vitro use, or the like. Additionally, there may be an analysis step where quality of the cells may be tested for potential use or perhaps to ascertain the potential end result of cell or tissue use.

In the past, treatment of biological cells started only when they have been received from transporting from a harvesting site. During the transportation, cells are subject to damaging environments, may grow bacteria, and may even have reduced viability, modified membrane structure or even be partially dead. At the processing facility, different collections may have to be combined to provide enough living cells for processing. Because these cells have been damaged and may contain bacteria or other biological contaminants, typically antibiotics are needed to be added to the cells.

In each of these steps it may be important, and perhaps even vital, to optimize the environment such that the cells (e.g., tissues, organs, or the like) are able to perform at a maximum when they are later utilized. It is well understood that it may be necessary to transport cells prior processing them, such as with cryopreservation. Cells may be collected at one location but processed and even frozen at another location. As but one non-limiting example, one can consider cryopreservation of stem cells that might be collected at a hospital location where a patient may be located, then the cells may be shipped to a second location such as a laboratory having cryopreservation expertise, then perhaps later utilized for transplant into a recipient at a third location. In another non-limiting example, sperm cells may be collected at a first location of the male animal and may need to be transported to a second location for a laboratory to process the cells for cryopreservation. Therefore, sperm cells may require transportation for up to about 24 hours before processing for cryopreservation. In addition, cells or tissues collected for medical/diagnostic analysis may require transportation to a facility where they are being analyzed or utilized. As yet another example, consider organ transplants. Often patients may be too ill to travel to the location of the organ harvest therefore said organ must be transported. If such cells (e.g., tissues) cannot undergo immediate cryopreservation, the transportation process may cause detrimental changes that could change the outcome of some diagnostic analysis, or ultimate use such as in cryopreservation of gametes.

In the first step, cells may be harvested from an in vivo source. Second, in order for the cells to be later successfully utilized in vivo, they should be free from harmful moieties including perhaps such items as bacteria or oxidants or the like that could be associated with the cells perhaps as a result of the harvesting procedure. Third, each distinct cell may need to be in a uniform environment perhaps contacted by the various beneficial components homogenously. Fourth, the materials may need to be surrounded by compounds that may help the molecular components of the cells survive the rigors of the full process. Fifth, it may be desirable that the materials utilized be compatible with the recipient source such that further processing or handling (and concomitant damage) may not be required.

Cells may be stored at a cooled temperature during transportation which may include storage at about 4° C., about 17° C. or even at an ambient temperature. In each of these cases, the cells (e.g., tissues or organs) may continue to respire and even actively metabolize. Doing so may result in the production of oxidants, other metabolites, and even metabolic biproducts that may immediately or even ultimately harm cells. In fact, for each about 10° C. decrease in temperature, most enzymes may show about 1.5 to about 2-fold decrease in metabolic activity. However, cooling may causes a lipid phase transition which may even cause cumulative damage to membranous structures, acidosis, mechanical trauma, free radical production, contracture, or even apoptosis, or the like. (See, for example, Rubinsky, Boris, 2003, “Principles of Low Temperature Cell Preservation,” Heart Failure Reviews, 8: 277-84, hereby incorporated by reference herein). In addition, transportation itself may cause movements, mechanical trauma, mixing or the like, that may result in the dissolution of oxygen or even superoxide into the solution which may ultimately harm the integrity of the cells.

In biological cell processing steps cells may undergo physical injury. The injury, such as mechanical trauma, can release endogenous damage associated with molecular patterns from the mitochondria (MTDs) that can prompt an immune response (See, for example, Qin Zhang, Mustafa Raoof, Chen Yu, Sumi Yuka, Tolga Sursal, Junger Wolfgang, Karim Brohi, Kiyoshi Itagaki, and Carl J. Hauser, 2010, “Circulating mitochondrial DAMPs cause inflammatory responses to injury,” Nature, 464: 104-07, each hereby incorporated by reference herein). While this may not cause an issue while the cells are held in vitro, once the cells may be in an in vivo environment, these MTDs may elicit neutrophil-mediated injury. In fact, these MTDs may prompt a response similar to sepsis perhaps because MTDs may be similar to bacteria (e.g., microbial pathogen-associated molecular patterns). Injection of MTDs into a rat liver intravenously seems to have caused marked inflammatory response as quickly as about 3 hours post injection (See, for example, Qin et al. 2010). One can then infer that a method to reduce the damage to collected cells which may decrease MTDs, may result in a more effective transfer of cells to a secondary environment. Considering stem cell harvesting and even transplantation for therapy: the elicitation of an immune response perhaps when injecting stem cells as healing therapy may be the anthesis of the anticipated and even desired response; therefore it may be imperative to develop a method to inhibit such responses such as by using natural and even non-injurious solutions. In addition, some antibacterial or bacteriostatic properties of a solution might be useful.

Using biological cells, perhaps in addition to the above issues, may include at least some dead cells from a donor source, or components of cells from a donor source which can provoke an inflammatory response in the host. (See, for example, Kono, Hajime, Dipti Karmarkar, Yoichiro Iwakura, and Kenneth L. Rock, 2010, “Identification of the Cellular Sensor That Stimulates the Inflammatory Response to Sterile Cell Death,” The Journal of Immunology, 184: 4470-78, hereby incorporated by reference herein.) As a non-limiting example, if stem cells are harvested, cleaned, then inserted into a secondary location, they may evoke an inflammatory response in the secondary location if some percentage of the cells have died during the processing. This response may have a number of consequences in the host location including perhaps, most detrimentally, rejection of the stem cells. Minimizing the death of cells in an in vitro solution may result in better acceptance of the intact, live cells when delivered in vivo.

In step one, biological cells may be retrieved with associated fluids and even materials. Such associated fluids may contain components that are, in fact, detrimental to the cells. Prior to transportation of such cells, it may be beneficial to inhibit the activity of the components, as might be the case with phospholipase A2 or similar phospholipases, which can be found in ejaculates from goats and other mammals (see, for example, Purdy, P. (2006), “A review on goat sperm cryopreservation.” Small Ruminant Research 63(3):215-225). Phospholipase A2 may lead to a pro-inflammatory response by hydrolyzing phospholipids (see, for example Iritani, A. and Y. Nishikawa (1963), “Studies on the egg-coagulating enzyme in goat semen: IV. On the position of yolk constituents attacked by the coagulating enzyme,” Jpn J Anim Reprod 8(4): 113-117 and Purdy (2006), each hereby incorporated by reference herein) which may damage the cells. Additionally, phospholipases might cause depolarization of mitochondria in cells such as sperm cells. The depolarization may result in decreased motility of sperm cells perhaps impairing the ultimate function of the cell. Compounds that are inadvertently collected such as Phospholipases may also negatively affect the solutions used for further processing of the cells perhaps resulting in multiplying its negative impact.

Other compounds such as bacterial cells may be collected in conjunction with the biological cells. These may be expelled from the source of the cells or may inadvertently be collected. Inadvertent collection may occur because of bacteria residing on the surface of the skin of an animal or perhaps because of bacteria residing on the equipment used for collection of the cells. Regardless of the source of contaminant cells, bacteria may be detrimental to the cells collected and also may be detrimental to the recipient of the cells such as but not limited to tissue or cells for transplantation as well as for cells used for reproduction. In addition, bacteria may be an integral part of the tissue or cells collected, as is well understood in the gut, rumen and other digestive tract organs. While in vivo, these bacteria live synergistically with the cells or tissues, once in an in vitro environment, the combination of the bacteria and cells or tissues may cause deterioration of said cells or tissues which may be injurious to their final purpose.

As mentioned above, cryopreservation may be a good method for preserving cells such as gametes, germ cells, unique cell lines, stem cells, umbilical cord tissues, bacterial, fungal, algal cells, and the like. Unfortunately, cryopreservation may also negatively affect the integrity of the cell, for example by causing changes to a lipid bilayer and even the proteins within the lipid bilayer as well as oxidative damage perhaps causing damage to the DNA and even organelles. Moreover, the lipid composition of the membrane can affect the success of cryopreservation.

Cryopreservation can affect the DNA, the mitochondria, or perhaps even other organelles in the cell. Such changes can be fatal to the cell. For example, in cryopreserving equine and bovine sperm cells, at least about 50% of the cells may be dead when said cells are thawed or perhaps returned to a non-frozen state. Similarly, in cells such as umbilical cord blood, depending on the technique used, only about 40% or less of the cells may be viable. But when cryopreserving boar sperm, which may have more phosphatidylethanolamine and sphingomyelin (see, for example, Johnson, L. A., et al. (2000) “Storage of boar semen.” Animal Reproduction Science 62(1): 143-172, hereby incorporated by reference herein), as much as about 100% of the boar sperm may be dead upon thawing, potentially due to this difference in phospholipid composition. As another example, different goat species may freeze with more or less success (about 0% to about 50% post-thaw motility) which may be due to membrane lipid composition.

The lipid composition of membranes may have a direct effect on the ability to cryopreserve a cell. In the past, attempts have been made with varying degrees of success to add cholesterol and similar fluidity inducing fatty acids to sperm cells. In addition, lipids have been added to culture medium when cells are growing.

Cryopreserved cells may be stored at around −20° C., about −80° C., or even −196° C. (e.g., liquid nitrogen storage). In the past, cells may be frozen in solutions that may contain sugars, lipids, antioxidants and perhaps even a cryoprotectant such as glycerol, DMSO, trehalose, methanol, or the like. While these may mitigate some of the damage and may have enabled cryopreservation for some species, sperm and cells from other species such as boars and felines, may still have too much damage to be effectively frozen commercially.

During each stage of the treatment process, cells may be surrounded by a non-uniform environment which may further damage the cells or even create more damage. For example, a cell may be in direct contact with sugar moieties but not with lipids nor glycerol. Alternatively, a cell may be in direct contact with another cell rather than a solution. Ultimate success of a cell when added to media may be in part dependent on exposure to each individual cell.

Solutions previously provided may focus only on one aspect or even one particular step of a process, but have never addressed the full process. For example, past methods to protect cells from damage may include transportation at reduced temperatures such as hypothermic preservation (above freezing), and perhaps even the addition of moieties as may inhibit, prevent or otherwise slow damage induced by negative compounds. Current moieties may include nonelectrolytes such as sucrose, raffinose, saccharoids, high molecular weight anions, buffers, glutathione for acidosis and the like (see, for example, Rubinsky, Boris, 2003, “Principles of Low Temperature Cell Preservation,” Heart Failure Reviews, 8: 277-84, hereby incorporated by reference herein.). Citrate might be used to delay activation of moieties such as phospholipase A2, but might be insufficient in some animals (see, for example, Roy, A. (1957), “Egg yolk-coagulating enzyme in the semen and Cowper's gland of the goat,” Nature 179(4554): 318, hereby incorporated by reference herein). Suppression of these bacteria might be achieved by chemical means such as perhaps by antibiotics. However, such compounds may be expensive and may be prohibited due to later interactions with recipient cells or tissues.

U.S. Pat. No. 6,864,046 may teach a method for collecting multiple cellular samples from an animal and may teach a method of diluting said cellular sample in a solution that may be specific to said species. However, this may not teach a method to modify the temperature of said cells, nor protecting cells from damage. Similarly, U.S. Pat. No. 8,685,563 (Ostermeier et al.) may teach a kit containing components necessary for cryopreservation but may not teach shipping of said cells in preparation for cryopreservation. US Pat. Pub. No. 2010/0196872 may teach a method to cryopreserve cells using phospholipids, surfactants, carbohydrates, freezing agents and perhaps buffers but does not address shipment or holding of cells at a temperature above freezing.

U.S. Pat. No. 6,982,172 may teach a method for cryopreservation of oocytes and embryos but may not discuss the method for obtaining or transporting said oocytes or embryos. US Pat. Pub. No. 20170367324 may teach a method to use vitamin B12 and alpha-ketogluterate for a sperm cell composition and diluent that can be held for perhaps 90 minutes. Unfortunately, in most instances this is insufficient time for shipment or transportation of cells from a collection site.

U.S. Pat. No. 5,358,931 may teach a method to protect cells using antifreeze polypeptides derived from polar fish species. This method may be applicable to cells at temperatures that are elevated relative to the cell which may be detrimental to some cell types.

U.S. Pat. No. 6,238,920 may teach a method of transporting bovine embryos in a non-frozen condition which may contain thiol compounds. Said thiol compounds may be detrimental to embryos at certain concentrations and therefore may not be optimal to cells or tissues and may not be effective in all species.

U.S. Pat. No. 6,395,305 may teach a method for increasing sperm survival using phospholipids but does not teach a method to ensure that sperm cells are exposed to a uniform environment of said phospholipids.

### DISCLOSURE OF THE INVENTION

The present invention includes a variety of aspects which may be selected in different combinations based upon the particular application or needs to be addressed. In one basic form, embodiments of the present invention provide synergistic continuity approaches to treatment and even preservation of biological cells which may encompass processes beginning at harvesting the biological cells to shipping the cells to use of the cells. In addition, embodiments of the present invention may include methods and systems which protect biological cells such as by providing uniform environments around each cell.

As to the goals of this invention, it may be understood that attempts at shipping biological cells have been fraught with hurdles including, but not limited to, inducing damage and artifacts that might be misinterpreted as being inherent in the cell rather than a function of shipping. Therefore, one goal of the present invention may include protecting biological cells from the hazards of shipping perhaps by treating cells based on their ultimate use.

Another broad goal of the present invention may be to provide pre-processing of biological cells during transportation so that when the cells arrive at the processing lab, the remaining processing may be reduced and the cells have been protected from the damages of transportation.

Yet another goal may be to provide holding media, perhaps specific for the ultimate use of the cells, that can be added to biological cells before or even during transportation.

Another goal of the present invention may be to provide appropriate processing for the transportation of biological cells which are to be cryopreserved for later use.

Yet another goal of the present invention may include providing a uniform cell environment to protect biological cells.

Naturally, further objects, goals and embodiments of the inventions are disclosed throughout other areas of the specification, drawings, and claims.

### BRIEF DESCRIPTION OF THE DRAWINGS

FIG. 1 provides a conceptual representation of various embodiments of the present invention.

FIG. 2 provides a conceptual representation of a biological cell in an uniform environment as may be understand in the various embodiments of the present invention.

FIG. 3 provides a conceptual representation of a biological cell in an environment as may be understand in the various embodiments of the present invention.

FIG. 4 provides a graph of the percentage of motility of boar sperm in shipping extenders including the average motility of cooled boar sperm prior to shipping (“pre_shipping_motility”), the average motility of cooled boar sperm upon arrival to a laboratory (e.g., after shipping) (“post_shipping_motility”), and the average motility of boar sperm after cryopreservation (“post_thaw_motility”).

FIG. 5 provides a graph of the total and progressive motility of rooster sperm held for 3 hours at either 4° C. or 22° C. in a control extender (BPSE with no additives (“Ct”)), 5% juice in BPSE (“juice—5%”), 5% Pulp Extract in BPSE (“pulp extract—5%”), and 0.25% leaf hydroglycerine extract in BPSE (“leaf hydroglycerine—0.25%”)

FIG. 6 shows a LC-MS chromatogram demonstrating the retention time of Sea Buckthorn extract indicating the presence of antimicrobials, anti-inflammatory, and antioxidants.

FIG. 7 shows non-limiting examples of pictorial representation of coagulation that can occur in egg yolk containing extenders if phospholipase A2 is not inhibited.

FIG. 8 shows a graph of a comparison of the area of coagulation of seminal plasma in a control (containing no phospholipase A2 inhibitor) versus in a seminal plasma plus a formulation (curcumin) containing an inhibitor which resulted in a statically significant decrease in coagulation of egg yolk particles compared to Control.

FIG. 9 shows a comparison of the number of bacterial colonies in shipped boar semen between control samples (untreated) compared to those treated with Formulation 1 or Formulation 2.

### MODE(S) FOR CARRYING OUT THE INVENTION

As mentioned earlier, the present invention includes a variety of aspects, which may be combined in different ways. The following descriptions are provided to list elements and describe some of the embodiments of the present invention. These elements are listed with initial embodiments; however, it should be understood that they may be combined in any manner and in any number to create additional embodiments. The variously described examples and preferred embodiments should not be construed to limit the present invention to only the explicitly described systems, techniques, and applications. The specific embodiment or embodiments shown are examples only. The specification should be understood and is intended as supporting broad claims as well as each embodiment, and even claims where other embodiments may be excluded. Importantly, disclosure of merely exemplary embodiments are not meant to limit the breadth of other more encompassing claims that may be made where such may be only one of several methods or embodiments which could be employed in a broader claim or the like. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

Embodiments of the present invention may provide methods and systems for protecting transported biological cells with synergistic continuity. It may be a full system perhaps considering five steps in processing of biological cells which may enable the decrease in damage of cells over the course of processing and even subsequent increase in post-processing cellular health. The synergistic continuity achieved by developing a fully integrated system, rather than a pieced system, may allow increased success of the cells, tissues or organs at the final point of use. By coordinating individual steps, one may decrease the processing time of the whole system thereby limiting damage to the cells. Compatibility within the system may allow protection at each individual step from components or compounds that may later cause a decrease in effectiveness. Much of the damage within in vitro systems may be cumulative. By limiting the initial damage, the accumulation of total damage can be decreased.

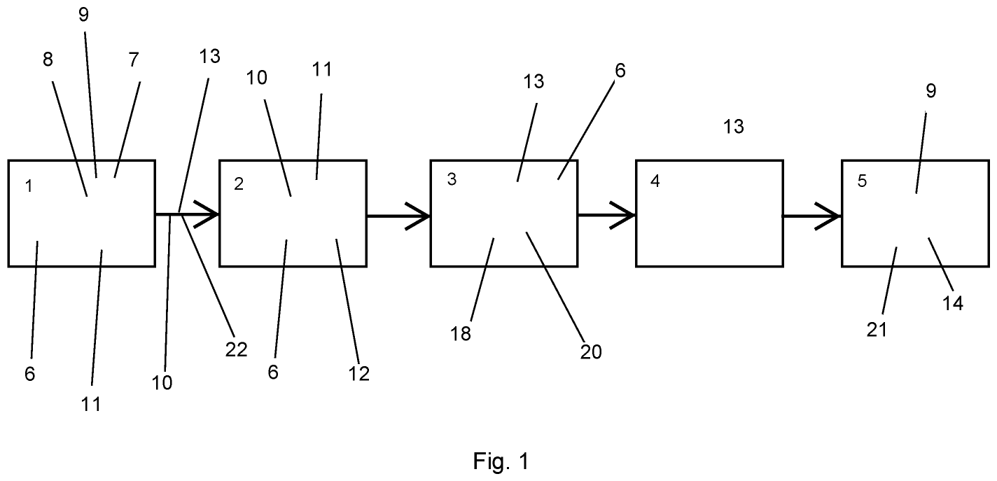

Embodiments of the present invention may consider the individual steps for processing but rather than optimizing the individual steps, optimizes the entire system for items that occur within each of the steps. As may be understood from the conceptual diagram of FIG. 1, a first step in a processing system may be a harvesting step (1) where a collection of biological cells (6) may be harvested from an in vivo source (7) , a second step may include transporting (2) biological cells, a third step may include hypothermic treatment (3) of said biological cells, a fourth step may include warming (4) of the biological cells, and perhaps even a fifth step of using (5) the biological cells.

Embodiments of the present invention may provide a method of protecting in vitro biological cells with synergistic continuity comprising the steps of harvesting a collection of biological cells from an in vivo source; preserving said collection of said biological cells based on a anticipated cell damage limiting regimen and a predetermined use; providing a holding media applicable for said anticipated cell damage limiting regimen and said predetermined use; adding said holding media to said collection of said biological cells; transporting said collection of said biological cells in said holding media based on said anticipated cell damage limiting regimen and said predetermined use; receiving said collection of said biological cells after said step of transporting said collection of said biological cells in said holding media; preparing said biological cells to be hypothermically treated; hypothermically treating said biological cells; warming said biological cells; and perhaps even using said biological cells for said predetermined use. In some embodiments, the present invention may provide a biological cell transport preservation composition comprising a collection of biological cells obtained from an in vivo source; a holding media to be applied to said collection of biological cells before transporting said collection of biological cells, said holding media applicable for an anticipated cell damage limiting regimen and a predetermined use of said collection of biological cells; and perhaps even a hypothermic treatment preparation media to be applied to said collection of biological cells after said step of transporting said collection of biological cells.

Other embodiments of the present invention may provide a method for maximizing viability of each cell in a collection of biological cells comprising the steps of harvesting a collection of biological cells from an in vivo source; establishing a uniform environment around each biological cell of said collection of biological cells; and perhaps even adding a phospholipase inhibitor to said collection of biological cells. In yet another embodiment of the present invention, it may provide a method for preserving harvested biological cells comprising the steps of harvesting a collection of biological cells from an in vivo source; creating a uniform environment around substantially each biological cell in said collection of said biological cells; hypothermically treating said biological cells; warming said biological cells; and perhaps even using said biological cells. Embodiments of the present invention may provide a method for maximizing viability of each cell in a collection of biological cells comprising the steps of harvesting a collection of biological cells from an in vivo source; preserving said collection of said biological cells; and even adding a phospholipase inhibitor to said collection of biological cells.

Embodiments of the present invention may provide a biological cell transport preservation composition comprising a collection of biological cells obtained from an in vivo source; a holding media to be applied to said collection of biological cells before transporting said collection of biological cells, said holding media applicable for an anticipated cell damage limiting regimen and a predetermined use of said collection of biological cells; and even a hypothermic treatment preparation media to be applied to said collection of biological cells after said step of transporting said collection of biological cells. Other embodiments of the present invention may provide a biological cell preservation composition comprising a collection of biological cells obtained from an in vivo source; a uniform environment established around each biological cell of a collection of said biological cells; and perhaps even a phospholipase inhibitor. In yet other embodiments of the present invention may provide a biological cell preservation composition comprising a collection of biological cells obtained from an in vivo source; a holding media to be applied to said collection of biological cells, said holding media configured to establish a uniform environment around each biological cell; and even a hypothermic treatment preparation media to be applied to said collection of biological cells after said step of transporting said collection of biological cells.

A collection of biological cells (6) may include, but is not limited to, cells, tissues, sperm, equine sperm, bovine sperm, caprine sperm, ovine sperm, porcine sperm, fowl sperm, ovaries, oocytes, embryos, organs, stem cells, genetically modified cells, artificially derived cells, any combination thereof, or the like. A collection of biological cells (6) may be harvested from an in vivo source (7) which may include, but is not limited to, mammal, human, rodents, equine, bovine, caprine, ovine, porcine, fowl, fish, shell fish, reptile, nephropidae, poikilothermic, aquatic vertebrates, or the like.

Once biological cells have been collected, embodiments of the present invention may provide preserving a collection of biological cells perhaps based on an anticipated cell damage limiting regimen (8) and even a predetermined use (9). As a non-limiting example, embodiments of the present invention may provide including an anti-inflammatory compound in a cryopreservation media (e.g., in step 3) that may be useful only when sperm cells are introduced to a uterine environment (e.g., in step 5). Therefore, embodiments of the present invention may consider the effectiveness of treatments such as the addition of media, addition of compounds, or the like that may be applied in an earlier or even a later step in a process thereby creating a synergistic continuity within a total system.

A predetermined use (9) may be a use of the biological cells at an end of a process. As a non-limiting example, a predetermined use may include insemination, implantation, culturing, research, diagnostic testing, replication, gamete preservation, genetic preservation, cryopreservation, reproduction, any combination thereof, or the like.

Embodiments of the present invention may include a method and formulation for protecting cells during further processing prior to processing for a predetermined use, such as freezing. For example, systems may include protecting cells during concentration steps such as centrifugation or filtration, during media changes or transitions, during temperature transitions, and the like. Embodiments of the present invention may be utilized to flush oocytes or even embryos and store them during transportation to a laboratory. The invention may also include devices appropriate to enable such utilization. It should be understood that synergistic continuity treatments at all steps or even levels may help to improve the health of cells, tissues or biological aggregates for future use and cryopreservation.

An anticipated cell damage limiting regimen (9) may include an expectation of what kind of cell damage may occur to a specific type of biological cells perhaps for a specific type of use and trying to limit such damage by modifying the process. Non-limiting examples of an anticipated cell damage limiting regimen (9) may include a reduction in cell damage perhaps caused from an aspect such as biological contamination, chemical contamination, contamination caused by invasive species, chemical residues, detergents, disinfectant residues, solvent compounds, organic compounds, photo activation, photo modification, improper handling, bacteria, fungi, mycoplasma, virus, any combination thereof, or the like. The present invention, in embodiments, may provide a holding media (10) perhaps applicable for an anticipate cell damage limiting regimen and even a predetermined use. A holding media may include components which may be tailored to limit cell damage for a system and may even be tailored for a predetermined use. A holding media (10) may include, but is not limited to, natural ingredients, non-animal derived components, microbial inhibitor, bacteriostatic compound, bactericidal compound, a compound that inhibits bacterial replication, antibacterial component, phospholipase inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Tetrapleura tetraptera extract, silibinin, phosphofructokinase, carnosine, lignans, fagaronine, ellagitannins, eschscholtzidine, saponin, any combination thereof, or the like. In some embodiments of the present invention, a holding media may be provided in a preservation kit (22).

A holding media (10) may be added to a collection of biological cells and a collection of biological cells in holding media may be transported. A holding media may be added immediately to biological cells after harvesting or may be added at some point after harvesting, perhaps before transporting. Transportation of biological cells may be any kind of transportation, including, but not limited to, shipping, automotive, carrier, or the like. A holding media, when added to biological cells, may allow the cells to pre-process perhaps during a preserving or even a transportation step. In some embodiments, a holding media may be a pre-processing media (12). Accordingly, embodiments of the present invention may provide a method to shorten the entire process by preparing the cells or tissues for the later steps, earlier in the process. This preparation may include optimally preparing cells to receive downstream (or later) processing. Accordingly, some embodiments of the present invention provide reducing an amount of in vitro exposure laboratory time spent on preparing cells to by hypothermically preserved or other processing. This may include decreasing an equilibration time of cells at a laboratory perhaps because the cells have been pre-processed earlier in the process, e.g., before or even during transportation. In embodiments, the present invention may provide that when using the biological cells, one may only need to utilize a single collection of biological cells perhaps because more of the biological cells have survived or are in a condition that can be used. In the past, a single collection of cells may not have provided enough for processing or use.

Some embodiments of the present invention may provide a system to decrease the number of cells or amount of tissue that may be required for harvesting, storage, and ultimately use which may provide a method to optimize the output of any given hypothermically treatment, such as cryopreservation, and storage system for finite resources such as cells, tissue or organs produced from a biological being.

Embodiments of the present invention may relate to the use of biological extracts or even plant extracts that may function as an anti-inflammatory, a natural antibacterial, an antioxidant, and an ice nucleator, or the like, as but a few non-limiting examples. A combination of extracts can be added to any variety of cell or tissue types perhaps thereby protecting the cell or tissue into which the cells or tissues are transplanted. In some embodiments, extracts may be derived from a plant source such as one that produces sap, berries, seeds, leaves, flowers, stems, bark, any combination thereof, or other parts that may be utilized to create an extract. These may be derived from any variety of plants such as the genus Hippophae, Parthenium, milk thistle or the like and may be derived from other genera of plants. Extracts may include a crude plant extract, a single source plant extract, a combination of extracts from more than one source, alcohol extracts, juice components, sodium hydroxide extracts, aqueous extracts, hydroglycerine extracts, any combination thereof, or the like. In addition, embodiments of the present invention may relate to the use of chemically defined media which may achieve a similar balance of active or even bioactive compounds. Such bioactive compounds may be derived from any number of sources.

As mentioned above, embodiments of the present invention may provide a crude plant extract, or combination of extracts from multiple sources which may include an anti-inflammatory compound, a compound that suppresses bacterial growth, a compound that inhibits bacterial replication or reproduction, antioxidants, membrane, or membrane phospholipid, stabilizing compounds, immune suppressant compounds, a compound that may act as an ice nucleator or as antiprotease compounds, or the like. In some embodiments, a system may include compounds derived from plants that may act in the above capacities or may even use chemically defined compounds, or yet may even include compounds known to function in such a capacity but in a novel combination of uses or applications, or even in portions of the process previously left untreated. Membrane stabilizing compounds may include extracts of Fagara zanthoxyloides, Olax subscorpioides, Hippophae rhamnoides, and Tetrapleura tetraptera. They may also include silibinin, sugars such as trehalose, phosphofructokinase, carnosine and similar extracts. Bacteriocidal compounds may include lignans, fagaronine, ellagitannins, eschscholtzidine, saponin, and various other phytochemicals, carbohydrates, or other bioactive compounds such as phenolics and organic acids or the like.

Embodiments of the present invention may include compounds such as heptadecanoyl ethanolamide, or steroid-like triterpenes that may be non-injurious to the recipient tissue and that may even help to allay an inflammatory response from the recipient tissue.

In some embodiments, compounds utilized may include naturally or artificially contain heptadecanoyl ethanolamide, triterpenes, steroid-like triterpenes, lipoglycopeptides, natural gums, natural resins, essential oils, tea tree oil, hyperenone A, hypercalin B, hyperphorin, phenolics, polyphenols, terpenes, flavonoids, alkaloids, propolis, spermidine, rutin, quercetin, coumarins, kaempferol, stigmasterol, campesterol, tocopherol, carotenoids, horseradish juice extract, tobramycin, any combination thereof, or the like, and such that may function as antimicrobial compounds or component. In addition, embodiments of the present invention may include an exogenous addition of compounds perhaps to increase a total concentration of antimicrobials, bacteriostatic or bacteriocidal compounds, perhaps to function in the various capacities. A microbial inhibitor may be a plant derived component.

Embodiments of the present invention may be understood to contain naturally occurring compounds that may function in more than one capacity. A compound such as a phenolic compound may function also as an antimicrobial compound.

In some embodiments, present invention may include a variety of widely and commonly used media which may be supplemented with plant extracts, or other antioxidants, lipids, sugars and the like to accomplish the functions of antimicrobials, anti-inflammatory, membrane stabilization and antioxidants.

Embodiments of the present invention could address a means of adding holding media, antioxidants, antimicrobials and anti-inflammatory compounds, and similar beneficial moieties, or the like perhaps at a metered rate during shipment such that the protection conferred by the moieties may not be depleted over the time of the shipment. This could include time release (e.g. time released compounds in a holding media), increased quantities (e.g., adding enough holding media to last throughout a transportation step), metered or even drip addition (e.g., adding additional holding media during a transportation step), and the like to keep biological activity perhaps at an optimum level. Moreover, embodiments of the present invention may include, within the system, the addition of various compounds at all points of the processes. In some embodiments, the invention may limit the changes of media within the processing perhaps by adding various compounds at the beginning to increase effectiveness throughout the entire process.

In embodiments of the present invention a holding media may include at least two components of: an antioxidant, a phospholipase inhibitor, membrane stabilizing agent, and a an antimicrobial agent, or the like.

Some embodiments of the present invention may provide a phospholipase inhibitor perhaps in a holding media or the like. A phospholipase inhibitor may be a phospholipase A2 inhibitor and may even be zinc, manganese, citric acid, and any combination thereof, or the like. These may be used in combination with plant extracts and/or other additives to a variety of cells, tissues, organs, or the like. The concentrations required may depend on the synergism with other additives in the system and may also depend on the amount and type of phospholipase present in the cellular collection exudate. In some embodiments, the present invention may provide natural phospholipase inhibitors such as plant extracts, cucurmin, extracts from Gingko biloba, Centella asiatica, Hippophae extracts as well as chemical phospholipase inhibitors pyrrolidone-based compounds, aristolochic acid, spermine and perhaps even neomycin sulfate which can also function as an antimicrobial compound, any combination thereof, or the like.

In embodiments of the present invention, an osmotic agent may be a plant extract.

Embodiments of the present invention may provide that preserving biological cells or even during transportation of biological cells may include cooling of the biological cells perhaps in a holding media such as with a cooler (11). This may include the ramping of cooling such that processing of the cells, tissues, and biologic extracts, may be available immediately upon arrival at a given facility location. It may include specialized methods to modify the temperature of the cells during treatment which may enable or optimize a process. Biological cells may be cooled to a temperature such as but not limited to between about 0° C. to about 37° C., about 4° C., about 10° C., and about 17° C., or the like. Cooling may be at a given rate, and perhaps to a given end temperature to facilitate positive membrane composition. Cooling of cells may be gently cooling perhaps in a way that may minimizes damage to the cells. Of course, the cooling rate may vary depending on the type of collection of cells, its lipid bilayer composition, volume, or the like. An example of a cooling rate for biological cells may be from between about 0.01° C./min to about 1° C./min. Embodiments of the present invention may also include a method for controlling the cooling rate as well as a prescribed cooling rate to optimize the health of said cells. It may also include specialized cooling methods to enable or even optimize the invention.

Embodiments of the present invention may provide holding cells at a given temperature perhaps to further enhance the functionality of the antimicrobials, anti-inflammatory, membrane stabilization and antioxidants. Some embodiments of the present invention may include compounds that are bacteriostatic, slowing the growth of bacterial compounds in place of, or bacteriocidal, eliminating the growth of bacterial compounds, in addition to an antimicrobial compound.

Embodiments of the present invention may provide receiving a collection of biological cells perhaps after transporting. Received biological cells may have a characteristic such as but not limited to reduced bacterial growth, increased bacteriostatic effect, and increased bactericidal effects, or the like, perhaps due to earlier treatment. The biological cells may then be prepared to be hypothermically treated such as with a hypothermic treatment preparation media (18) which may include various components such as a cryoprotectant when a hypothermic treatment may be cryopreservation. A hypothermic treatment preparation media (18) may include hypothermic components such as but not limited to antibiotics, natural ingredients, non-animal derived components, microbial inhibitor, bacteriostatic compound, bactericidal compound, a compound that inhibits bacterial replication, antibacterial component, phospholipase inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemical s, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Tetrapleura tetraptera extract, silibinin, phosphofructokinase, carnosine, lignans, fagaronine, ellagitannins, eschscholtzidine, saponin, any combination thereof, or the like. In some embodiments, the present invention may provide adding an antioxidant to biological cells that are to be hypothermically treated such as in a media, which may include but is not limited to, allene oxide synthase, phenolics, flavonoids, ascorbic acid, tocopherols, carotenoids, tannins, butylated hydroxyani sole, butylated hydroxytoluene, tert-butylhydroxyquinone, propyl gallate, and compounds, plant derived or synthetic, sufficient to reduce or scavenge reactive oxygen species superoxide, hydroxyl, peroxyl, alkoxyl, nitric oxide, singlet oxygen, hydrogen peroxide, any combination thereof, or the like.

Embodiments of the present invention may provide that a hypothermic treatment (20) may include, but is not limited to, cooling, cryopreservation, freeze-drying, lyophilization, vitrification, or the like.

When preparing biological cells to be hypothermically treated such as with a hypothermic treatment preparation media, less antibiotics may be utilized than for biological cells that have not been treated earlier in the process, such as before transporting or the like. Use of less antibiotics may include but is not limited to less than about 50 IU/ml penicillin, less than about 100 IU/ml penicillin, less than about 50 μg/ml streptomycin, less than about 100 μg/ml streptomycin, less than about 500 μg/ml streptomycin, less than about 500 IU/ml penicillin, less than about 150 ug/ml lincomycin, less than about 300 ug/ml spectinomycin, or the like. In some embodiments, antibiotics that may be added to biological cells, such as cells that have been shipped, may be substituted, at least in part, with a plant extract. This may include, but is not limited to, substituting about 10% of the antibiotic with a plant extract; substituting about 20% of the antibiotic with a plant extract; substituting about 30% of the antibiotic with a plant extract; substituting about 40% of the antibiotic with a plant extract; substituting about 50% of the antibiotic with a plant extract; substituting about 60% of the antibiotic with a plant extract; substituting about 70% of the antibiotic with a plant extract; substituting about 80% of the antibiotic with a plant extract; substituting about 90% of the antibiotic with a plant extract; substituting about 100% of the antibiotic with a plant extract, or the like.

In some embodiments, the present invention may provide maintain an in vivo redox potential within biological cells perhaps during the preservation or even the transportation steps, or the like. This may be accomplished with a combination of lipid soluble and aqueous antioxidants perhaps in a holding media or the like. These antioxidants may be a plant extract.

In some embodiments, the present invention may include method and systems that may leverage a cooling & shipping methodology and may extend it to a methodology for cryopreservation, and or preparation for cryopreservation.

The combination of proper cooling, holding media, and even antioxidants can facilitate the shipment of cells or tissues for diagnostic testing, cryopreservation, replication or other such methodologies as may require cells, tissue, biologic materials to arrive intact and biologically active.

The present invention, in embodiments, may be utilized to optimally prepare cells to receive downstream (or later) processes such that the cells may benefit maximally from other treatments that may occur. In some embodiments related to harvesting cells for cryopreservation, a small amount of cryoprotectant may be added to biological cells early in the process which may enable the cells, tissues or organs to be less damaged, may have a longer equilibration period, may require less cryoprotectant after transportation, and may even result in better cellular health when thawing. Accordingly, embodiments of the present invention may provide an improved post-warm cellular health (21) for the biological cells perhaps after a hypothermic treatment. An improved post-warm cellular health may be analyzed by pregnancy rates such as when sperm may be frozen and then thawed. An improvement to cell health after thawing may be compared to pregnancy rates of thawed sperm which have not been treated earlier in a process. For example, an improved post-warm cellular health may be greater than about 25% pregnancy rate artificial insemination of post-warmed bovine sperm cells.

In embodiments, a cryoprotectant may include but is not limited to glycerol, glycine, dimethylsulfoxide, proline, modified betaines, glycinebetaine, dimethyl sulphoniopropionate, cyclohexanediol, methyl formamide, dimethyl formamide, ethylene glycol, trehalose, concentrated complex sugars, tree sap, concentrated sugars, penetrating cryoprotectants, non-penetrating cryoprotectants, plant extracts, any combination thereof, or the like.

Embodiments of the present invention may provide that a system may be used with any step for processing of cells. For example, a preservation step may be accomplished by a system (13) such as but not limited to microfluidics, flow cytometry, or the like systems. In addition, or even alternatively, a preparing step may be accomplished by a system (13) such as but not limited to microfluidics, flow cytometry, or the like systems.

Embodiments of the present invention may provide a composition of fatty acids perhaps in the range of about 0.5% to about 10% v/v to provide stabilization to membranes to protect the cells from exposure to oxidative compounds, to prevent cell disruption, cell rupture and expulsion of cytoplasmic compounds that may be injurious to adjacent, intact cells, individually and any combination thereof.

Other embodiments of the present invention may provide a method or device to limit oxygen exposure of the cells and surrounding solutions.

Some embodiments of the present invention may include a step post-collection that limits the type or variety of cell. Sex selection of cells may be a step between collection and hypothermic preservation. Additionally, there may be some selection of cells including flow cytometric selection of cells of a specific type such as selection of stem cells containing various markers. The invention should be understood to encompass the media that the cells may be collected or processed within as part of the synergistic continuity for optimum final product.

Embodiments of the present invention may include methods for freezing or alternatively freeze-drying, lyophilization, vitrification or other preservation steps. Also, the invention may also include the addition of the cryoprotectant which can occur in either a stepwise addition or a single step as is necessary for the particular cell or tissue type. This addition may further enable rapid freezing after transportation. The invention may include traditional cryoprotectants such as glycerol, glycine, DMSO (dimethylsulfoxide), methyl formamide, dimethyl formamide, ethylene glycol, or may include alternative cryoprotectants such as may be derived from plants or other compounds that serve to create a hypertonic environment and perhaps desiccate or otherwise prepare the cells for cryopreservation such as proline, modified betaines perhaps glycinebetaine, dimethylsulphoniopropionate, cyclohexanediol, and perhaps even trehalose or tree sap

A method and formulation for protecting cells during preserving, transporting, processing, hypothermically treating, or the like may include methods to modify the immediate environment surrounding the cell. Embodiments of the present invention may provide an ability to add specific extracts perhaps customized to an innate lipid composition of the cell perhaps to be cooled and/or cryopreserved, have it exposed to the cell in such a way as to make it be more effective, and may thereby protect the integrity of the cell, or tissue with respect to its functionality. Some embodiments of the present invention may provide an ability to change the membrane composition, and/or to adjust the environment directly surrounding the cells to compensate for deficits in the membrane composition. In other embodiments, the present invention may provide a medium surrounding the cell that may be customized to the cell and may be uniform to all cells. Embodiments of the present invention may provide an ability to retain a uniform environment surrounding the cell until such a time as it may be desirable to release said environment. Accordingly, some embodiments of the present invention may provide creating a uniform environment (15). This uniform environment may be created with a system such as but not limited to microfluidics, encapsulation, creating liposomes, creating a micelle, creating a biological cage structure, any combination thereof, or the like. A uniform environment (15) may provide encapsulation of cells.

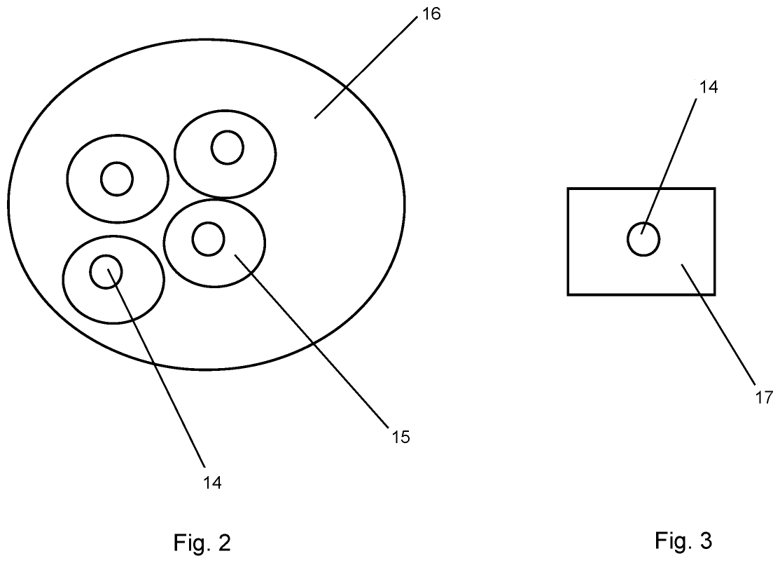

As may be understood from the conceptual representation provided in FIG. 2, a biological cell (14) may be encapsulated or even surrounded by a uniform environment (15) which may then be surrounded by a media (16). FIG. 3 shows an example of a conceptual representation of a cage-like structure (17) around a biological cell (14). A cage-like structure may be a three-dimensional complex.

Embodiments of the present invention may relate to the ability to add compounds which may compensate for the endogenous (or native) lipids which may be inhibiting or conversely enabling membrane fluidity. Such lipids may be external to the cell or may be attached to the cell or may be incorporated into the cell. The choice of interaction of the lipids with the membrane may be dependent on the cell type, the ultimate goal for use of the cell, the type of storage media, the type of desiccation and/or cryopreservation method utilized or any combination thereof. Embodiments of the present invention may relate to a method of creating and maintaining such a customized environment perhaps by encapsulating the cells.

Some embodiments of the present invention may include any commonly identified membrane lipids, may be free fatty acids, may contain phosphoglycerides, sterols or sphingolipids, and may also contain membrane proteins, salts, agarose, or other materials. Such materials may interact with the phospholipid head group to form a cage-like structure (17) that may stabilize the lipids and/or lipid rafts in the cell membranes. A uniform environment may include a compound such as but not limited to membrane lipids, glycolipids, cholesterol, free fatty acids, phosphoglycerides, sterols, sphingolipids, membrane proteins, salts, agarose, any combination thereof, or the like.

Embodiments of the present invention may contain an unusual distribution of lipids such as linolenic acid (18:3) at approximately 40%, linoleic (18:2) at about 15% and palmitic at about 20%. Lipids may be commonly found in the species of the plant or animal or may be lipids not found within the family, genus or species. Lipids may also be from a different phylogenetic kingdom, such as plants with animal cells and the converse. In some embodiments, a blend of lipids, free fatty acids, phospholipids, and cholesterol may be added to biological cells which may be optimally beneficial to an individual cell type and a cell derivation.

Some embodiments of the present invention may provide the addition of glycolipids, cholesterol and proteins as part of the addition. It may be understood that the addition could be aqueous and/or lipid-based, and could contain a combination of compounds.

The present invention, in embodiments, may include methods to encapsulate the lipids such that they can be integrated into the membrane. Such encapsulation may include liposomes, or similar methods to enable the separation, the addition and the incorporation of the lipids as the invention may dictate.

The present invention, in some embodiments, may include a specific energy to be added to the lipids to enable the lipids to be added without interfering with the cryopreservation process.

Embodiments of the present invention may include a micellular structure, a lipid bilayer or monolayer surrounding a core. The core may also be lipids, or other beneficial materials such as antioxidants. Such structure may serve to deliver the lipids to the cell or to enhance the functionality of such cells.

Some embodiments of the present invention may provide a method for analysis prior to cryopreservation to allow individual optimization of the formulation. Such optimization may occur on the phylogenetic family, genus, species or individual animal level.

The present invention, in embodiments, could include a variety of salts or other compounds or group of compounds, lipids, salts, proteins, BSA proteins, combinations thereof, or the like, that function to create a complex that may function as a cage, a support or a three-dimensional framework to stabilize the membrane such that membrane components are not externalized prematurely. Examples may include phosphatidyl serine, agarose, which may be normally found in the inner leaflet of the membrane but externalized during cryopreservation.

The present invention, in some embodiments, may include the creation of a ‘bubble’ or ‘blanket’ of beneficial compounds surrounding the cell which may limit changes in the membrane composition of the cell itself.

Embodiments of the present invention may provide a specialized method to optimize or standardize the environment surrounding the cells during treatment or exposure to the invention. Such a method may include a method to encapsulate a small grouping of the cells into a limited uniform environment. Such methods may include a microfluidic type system to create a microenvironment. Encapsulating a biological cell may be accomplished by micellular structure; a lipid layer, a lipid monolayer, a lipid bilayer, or the like. The microenvironment may include antioxidants, plant lipids, egg yolk, and/or any number of a variety of moieties which may be known to be beneficial to cells in addition to the lipids in which to expose the cells. The microenvironment may include any of the aforementioned attributes such that the continuity of the system and indeed of the cellular or tissue environment is maintained over the entire course of processing. The creation of the microenvironment may be achieved by methods similar to, or including the ‘Dolomite Microfluidics Droplet System’ or perhaps a system as may be made by ‘Elveflow.’ While said system may not have been previously used to encapsulate cells such as sperm for use in artificial insemination, this system may provide one method for development of an appropriate microenvironment to encapsulate a small number of cells.

A grouping of said microenvironments may then be further surrounded by the appropriate media to create a suitable environment for processing. Such microenvironment may include some type of external support that may provide a method to maintain the microenvironment until such a time that it may be desirable to release the cells from said microenvironment. As but one example, the microenvironment (15) may be maintained by agarose. The microenvironment may be intact at cooled temperatures such as about 4° C. or at frozen temperatures such as about −20° C., or maybe as cold as about −196° C., as may be imparted by liquid nitrogen storage. Embodiments of the present invention may provide processing a microenvironment including but not limited to cooling said microenvironment to between about 0° C. to about 37° C., cooling said microenvironment to about 4° C., cooling said microenvironment to about 10° C., cooling said microenvironment to about 17° C., freezing said microenvironment, freezing said microenvironment to about −20° C., and freezing said microenvironment to about −196° C., or the like. The microenvironment may be released at temperatures such as about 20° C. or may be intact until the temperature reaches about 37° C.

Embodiments of the present invention may be applicable to a wide variety of commonly utilized media, buffer or extenders for cryopreservation, shipping, thawing, tissue preservation cells, tissues or organs.

The present invention, in embodiments, may include a method to promote integration of the lipids into the cellular lipids or may enable distinct separation of the lipids. Such methods may include creating liposomes that increase fusion of lipids with membranes, or may include methods to inject lipids into membrane, perhaps via a molecular method.

Lipids may include lipids, free fatty acids, phospholipids, proteins, glycoproteins, lipoproteins, and other compound containing lipids and the like as might be described above. Compounds may include lipids and lipid components but may also include as sugars, salts, proteins, compound molecules, phytochemicals, secondary metabolites of plants, and similar moieties that might all function in a similar, or substantially similar, manner.

Embodiments of the present invention may provide a system for analysis of cells, tissues or organs, to verify use either prior to use or for a subsample retained to analyze after the majority of cells have been utilized. Such analysis may include analysis of motility, analysis of membrane quality, analysis of oxidation within the cell, tissue or organ, analysis of

[symbol]

Embodiments of the present invention may be achieved using a microfluidics systems that treats cells or small groups of cells nearly individually thus ensuring optimal exposure to a uniform environment. The disclosed invention may also utilize a microfluidics system to encapsulate the cells in a discrete, optimum environment.

As mentioned before, embodiments of the invention may provide a method to shorten the entire process by preparing the cells or tissues for the later steps, earlier in the process. This preparation may include optimally preparing cells to receive downstream (or later) processing. For example, if phospholipase may be inhibited, then the exposure of the cells and cellular components to egg yolk later in the process, may have no negative reaction. The exposure of cells and cellular components not treated with a phospholipase inhibitor until egg yolk is present (in step 3) may cause a 50% reduction of coagulation whereas treatment in step 1 may cause a 80% reduction in coagulation of egg yolk.

### EXAMPLE 1

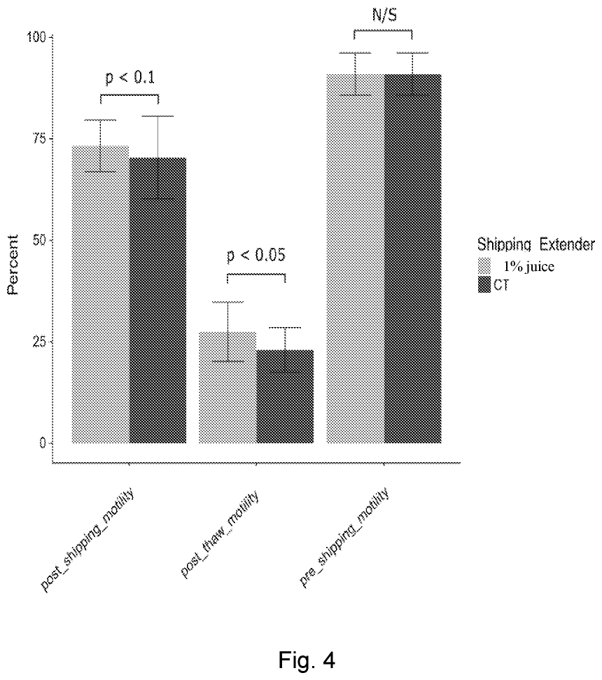

The first step in creating synergistic continuity is the immediate treatment of the cell as it transitions from an in vivo to an in vitro environment. This experiment demonstrates the importance of this immediate treatment using antimicrobial, antioxidant and anti-inflammatory compounds on the final product (post-thaw motility in FIG. 4). This impact demonstrates the need for synergistic continuity (maintaining a high level of antioxidants, antimicrobial and anti-inflammatory compounds) within the system to obtain a cell ready for its predetermined use. All cells were frozen with the presence of these compounds and therefore this demonstrates importance of the continuity of these compounds.

An experiment was conducted to assess the effectiveness of adding plant extracts in a shipping extender to assess post-thaw sperm quality of 10 boars. For each of the 10 boars sperm was collected then diluted into 1 of 2 solutions at a rate of 1:1 (v/v), in either extender alone (Control (“CT”): commercial extender AndroStar® Plus; Minitube) or in an extender containing 1% (v/v) Sea Buckthorn juice extract in extender AndroStar Plus™ (1% juice). Motility was first analyzed, then sperm was cooled to 17° C., and transported approximately 6 hours (e.g., about 330 miles). Upon arrival at the lab, samples were assessed for motility, then centrifuged, the supernatant removed, and the sperm pellet was resuspended in the same freezing media (USDA lactose egg yolk extender). Sperm was frozen per USDA protocol. A frozen straw of each treatment was thawed at 70° C. for 8 seconds then analyzed for motility using a CASA. This was replicated two times.

In FIG. 4, the average motility of the boar sperm is graphically shown for cooled boar sperm prior to shipping, after shipping, and after thawing from freezing. As expected, the motility does not differ before transportation. In the laboratory however, by addressing the initial environment in a way that is compatible with the final product, and by limiting the oxidants and bacterial load during transportation, the final product is improved. Improving the pre-freeze health of the sperm with the addition of plant extracts, causes a statistically significant improvement in the final product (post-thaw motility). The plant extracts provided antimicrobial, anti-inflammatory and antioxidant effects. When assessing post-thaw sperm health, the improvement imparted by transporting in Sea Buckthorn juice, are also statistically significant. The addition of the extract prior to transportation resulted in improved post-thaw sperm health.

### EXAMPLE 2

Synergistic continuity is important in frozen cells (Example 1) as well as cooled cells (this example). This experiment assessed the impact of antioxidants, antimicrobials and anti-inflammatory compounds in the initial holding or shipping media (step 1). The presence of antioxidants and antimicrobials results in a 11-14% (depending on combination of antioxidants and antimicrobials) improvement over control which contained no antioxidants or antimicrobial compounds. Presumably the healthier cells prior to final hypothermic treatment will result in healthier final cells.

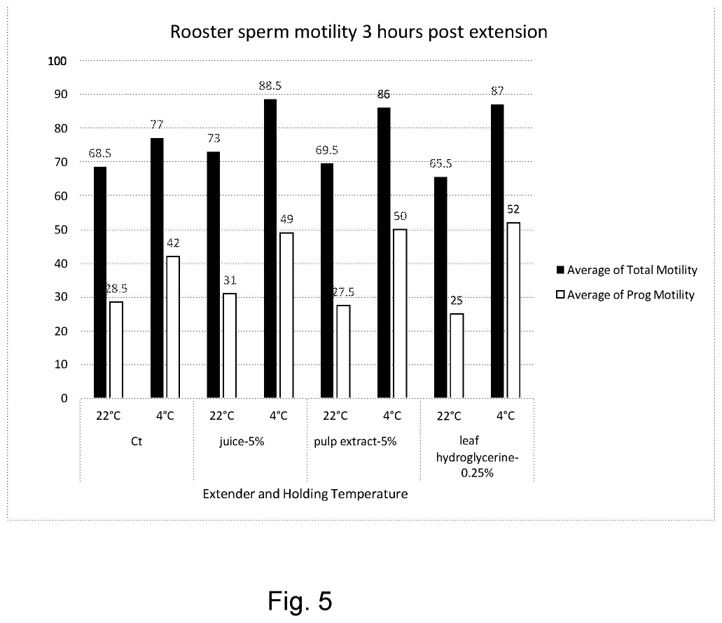

In order to assess the effectiveness of three extracts on the holding of cells for future hypothermic storage, semen from one rooster was collected using abdominal message and extended 1:1 (v:v) with Beltsville Poultry Semen Extender (“BPSE”) then transported back to the lab. Sperm was extended into treatments at a concentration of 2×10⁹ sperm/ml. Treatments included 5% juice in BPSE (“juice—5%”), 5% Pulp Extract in BPSE (“pulp extract—5%”), 0.25% leaf hydroglycerine extract in BPSE (“leaf hydroglycerine—0.25%”), and control BPSE with no additives (“Ct”). Aliquots of each sample were stored for 3 hours at either 4° C. or 22° C. then analyzed for motility and progressive motility using a computer assisted sperm analyzer (CASA). FIG. 5 and Table 1 illustrate the improvement in motility as compared to the control demonstrating that the improvement with a variety of transportation media containing antioxidants, anti-inflammatory, and antimicrobial compounds. Presumably the improvement within the first step of the continuous process will result in improvement in the end product.

TABLE 1
Changes in post-thaw motility depending on shipping treatment.

| | Total | Total | Percent | Percent |
|---|---|---|---|---|
| | Motility | Motility | improvement | improvement |
| Treatment | 22 C. | 4 C. | over control | over control |
| juice-5% | 73 | 88.5 | 6.5 | 14.9 |
| pulp extract- [symbol] | 69.5 | 86 | 1.4 | 11.7 |
| leaf | 65.5 | 87 | — | 12.9 |
| hydroglycerine - | | | | |
| Control | 68.5 | 77 | — | — |

[symbol]

indicates data missing or illegible when filed

### EXAMPLE 3

Sea Buckthorn extracts as utilized in some of the aforementioned experiments were analyzed for antioxidant, anti-inflammatory compounds, and antimicrobial compounds. In addition to antioxidants, Sea Buckthorn is known to contain Triterpenes, which are steroid-like molecules that can function as anti-microbial agents (See Baoru Yang, Riina M. Karlsson, Pentti H. Oksman, and Kallio, H. P., “Phytosterols in Sea Buckthorn (Hippophaë rhamnoides L.) Berries: Identification and Effects of Different Origins and Harvesting Times,” (2001), doi:10.1021/JF010813M, 5620-5629 and Mokoka, T. A. et al., “Antimicrobial activity and cytotoxicity of triterpenes isolated from leaves of Maytenus undata (Celastraceae),” BMC Complement. Altern. Med. 13, 111 (2013), 9 pages, each hereby incorporated by reference herein). Indeed, Sea Buckthorn berries have antimicrobial properties which may be in part due to the Triterpenes (See Michel, T., Destandau, E., Le Floch, G., Lucchesi, M. E. & Elfakir, C., “Antimicrobial, antioxidant and phytochemical investigations of sea buckthorn (Hippophae rhamnoides L.) leaf, stem, root and seed,” Food Chem. 131, 754-760 (2012), hereby incorporated by reference herein).

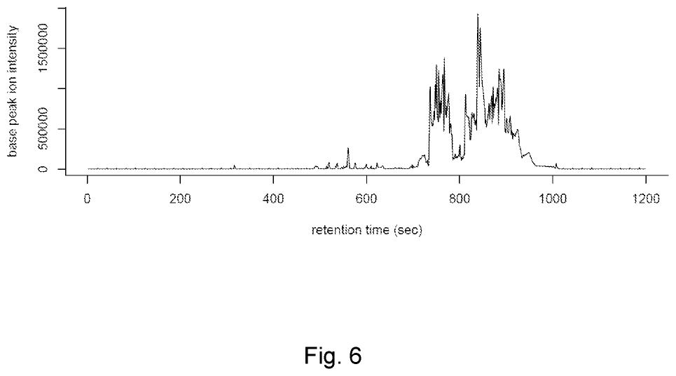

LC-MS research on Sea Buckthorn identified a set of compounds that support the aforementioned properties as shown in FIG. 6 and Table 2. A number of potentially anti-inflammatory compounds were also identified in this dataset. From these data and the data present in literature, Sea Buckthorn extracts can be described as having antioxidant, antimicrobial, and even anti-inflammatory activities that can be useful in a synergistic continuity of processing cells including transportation.

TABLE 2
Identified compounds in laboratory prepared extracts with biological
activities (see FIG. 6 for the chromatogram).

| COMPOUND | FUNCTIONALTY/TYPE |
|---|---|
| Alatanin | Antioxidant |
| B-Carotene | Antioxidant |
| Chlorobiphenyl-chloroeremomycin | Anti-microbial |
| diiodoplatinum(2+); [4-(pyridin-2- | Anti-microbial |
| ylsulfamoyl)phenyl]azanide | |
| lipoglycopeptides | Anti-microbial |
| Heptadecanoyl ethanolamide | Anti-inflammatory |
| steroid-like triterpenes | Anti-inflammatory |

### EXAMPLE 4

An experiment was conducted to assess the effects of adding compounds to inhibit harmful moieties that are inadvertently collected when a collection of biological cells are obtained. A portion of synergistic continuity is anticipating the final use of the cell in order to determine which compounds may be detrimental to the final product then inhibiting or slowing the effects of said detrimental compounds in the early steps of in vitro processing to result in a superior final product. As but one example of a detrimental compound is the presence of phospholipase A2 in goat sperm seminal plasma expelled during ejaculation. The following experiment demonstrates the effects of early inhibition of phospholipase A2 and the positive longer-term impact. A second portion of synergistic continuity is to utilize compounds that might have multiple effects within the system. It should be understood that phospholipids function in inflammation as well as oxidative stress therefore phospholipase A2 inhibition is decreasing two of the collective harmful moieties that might affect successful predetermined cellular use.

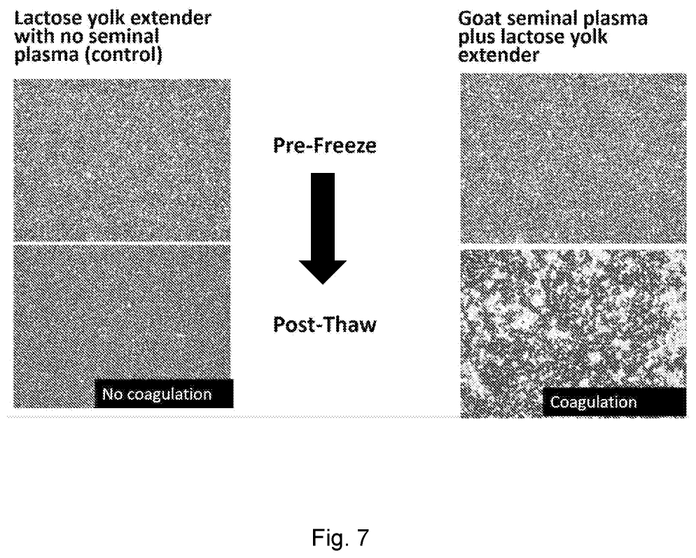

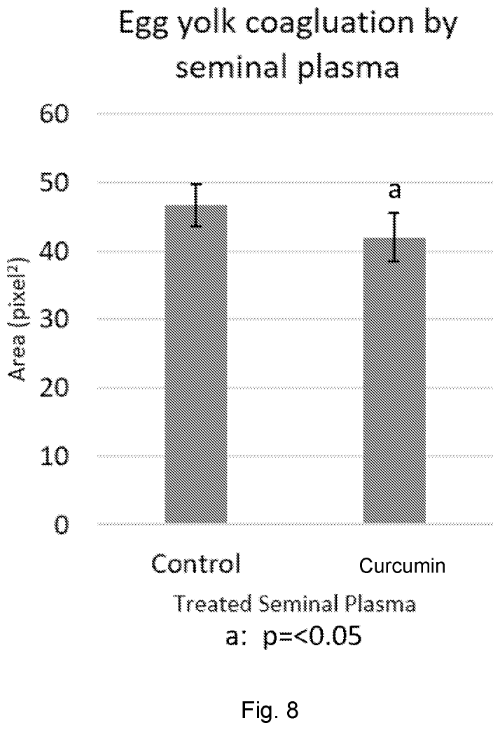

An egg yolk extender was prepared substituting lactose (Trizma base 131.5 mM, Lactose 73 mM 40% v:v egg yolk), without citric acid and pH was adjusted to a standard extender pH of 7. Semen from 3 bucks was collected using an artificial vagina. The ejaculate was centrifuged for 5 min at 1000×g. The supernatant (seminal plasma) was removed, then 1 part seminal plasma was added to 1 part extender to create a 20% yolk solution. Extender was either lactose yolk extender alone or lactose yolk extender modified to contain the phospholipase inhibitor curcumin as found in Sea Buckthorn (see Gupta et al. Molecular and Cellular biochemistry October 2006 290:193, hereby incorporated by reference). Representative pictures were taken of each solution, then the 22° C. solutions of seminal plasma plus the various extenders were frozen to −80° C. and allowed to remain frozen for >24 hrs. Samples were thawed by incubation in 37° C. water bath for 1 minute, then representative pictures were taken. For each picture, the negative space (the white area visible between droplets) was measured using ImageJ software by adjusting the image threshold using “Auto” setting then analyzing particle size (see Rasband, W. S., ImageJ, U.S. National Institutes of Health, Bethesda, Md., USA, https://imagej.nih.gov/ij/, 1997-2018, hereby incorporated by reference). The mean particle size was recorded and the images were analyzed in triplicate. FIG. 7 shows representative pictures of coagulation that can occur in egg yolk containing extenders if phospholipase A2 is not inhibited. The left column is a non-limiting example of a pictorial representation of the lactose yolk solution without seminal plasma (control) pre-freezing and post-freezing. One observes that there is no significant change in the size of the egg yolk lipid droplets, and no coagulation has occurred. The right column shows a non-limiting example of lactose egg yolk solution to which seminal plasma was added. Before freezing there is no coagulation, and yet as can be observed in Table 3, the pre-freeze seminal plasma treated with 0.2% plant-derived curcumin is less coagulated than those without curcumin it is not different from control. Post-freeze (coagulation (large, dark particles) are a visible (see non-limiting example in FIG. 7 right column). This is a visual representation of coagulation that can occur in egg yolk containing extenders if phospholipase A2 is not inhibited. The amount of egg yolk coagulation induced by endogenous phospholipase in the seminal plasma is significantly greater if no inhibitor is present (FIG. 8).

TABLE 3
Aggregate data from a single pygmy buck demonstrating increased
coagulation when cucurmin is absent.

| | Pre- | Post- | |
|---|---|---|---|
| | Freeze | Thaw | |
| | Area | Area | Coagulation |
| Extender/seminal plasma | (pixel²) | (pixel²) | rank |
| Lactose yolk extender with NO | 33.7 | 24.1 | — |
| seminal plasma | | | |
| goat seminal plasma plus lactose | 36.1 | 43.6 | 2 |
| yolk extender | | | |
| goat seminal plasma plus lactose yolk | 26.3* | 35.6* | 1 |
| extender treated with curcumin | | | |

(*p < 0.05)

The above example demonstrates the importance of inhibition of harmful moieties collected in vivo early in the process of hypothermic preservation. Treatment of the in vitro cells immediately upon collection has a positive impact both on the collection media (pre-freeze, Table 3) as well as on the cryopreservation media (post-Thaw table 3 and FIG. 8), again demonstrating the importance of synergistic continuity, consideration of the end product and detrimental moieties, within the entire process.

### EXAMPLE 5

Consideration of harmful moieties present in the cellular milieu is of essence in the synergistic system and to provide continuity to a productive final product. One such harmful moiety might include bacteria. Bacteria take time to replicate therefore suppression of bacterial growth or elimination of the bacteria, through some means at the beginning of the process will result in improved use of the cells. Bioactive compounds isolated from Sea Buckthorn were tested for effectiveness on post-thaw health when used immediately after in vitro collection of the cells.

Handling of cells may be increase, cause or inadvertently collect bacterial contamination. As a non-limiting example, a split ejaculate study was performed using cooled boar semen. Semen was collected from 12 boars using the digital pressure method and the gel fraction was removed. Semen was extended to 1.25×10⁹ sperm/dose (75 ml doses) in 3 different treatments: AndroStar Plus (MOFA global) (Control), AndroStar Plus with 300 ppm complex phenolic polymers as antimicrobial compounds isolated from berries (Formulation 1), and AndroStar Plus with Formulation 2 containing 200 ppm complex phenolic polymers plus 100 ppm organic acids isolated from berries (Formulation 2). Samples were sealed and shipped at 17° C. overnight to a laboratory for analysis.

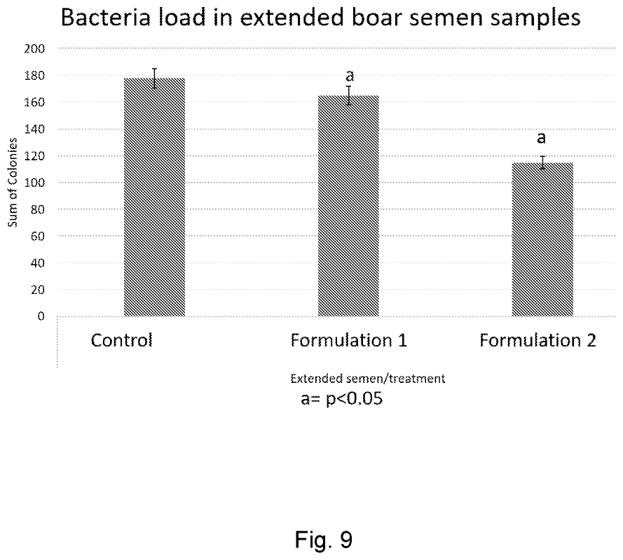

After shipping, samples were plated on blood agar plates in duplicate and incubated at 37° C. for 48 hours. The total number of colonies for each treatment was counted. Treatments were compared statistically using pairwise comparison ANOVA. As shown in FIG. 9, both plant extract treatments (Formulation 1 and 2) were statistically improved compared to control indicating a suppression of microbial growth in the treated samples.

Suppression of microbial growth early in the process will result in sperm cells that are not acrosome reacted, have greater intact membrane and fewer oxidants within the solution. All of these result in a greater concentration of healthy cells after hypothermic preservation. These data demonstrate the effectiveness of synergistic continuity.

### EXAMPLE 6

To further demonstrate the importance of synergistic continuity, a split ejaculate study was performed using buck semen. The addition of plant extracts was assessed throughout each step of the cryopreservation process to benchmark the importance of protecting cells in a complete system.

Sperm was collected from 2 bucks (Step 1) and immediately diluted in a standard medium (Control), or a medium containing antioxidants, membrane protectants, and bacteriostatic compounds (Treated). The cells were held for 24 hours at 17° C. to simulate shipping (Step 2) then cryopreserved (Step 3). Cryopreservation media was either Control medium (industry standard Egg Yolk Citrate) or Treated medium (Egg yolk Citrate plus antioxidants, membrane protectants, and bacteriostatic compounds). Samples were loaded into 0.25cc straws and frozen over nitrogen vapor for 20 minutes before plunging in liquid nitrogen. Straws were stored for a minimum of 24 hours, thawed in 37° C. waterbath for 1 minute and analyzed for motility at 0 and 3 hours post-thaw. Samples were also analyzed using flow cytometry at 0 and 3 hours post thaw for membrane and acrosome integrity using SYBR Green/Propidium Iodide and AlexaFluor 647 respectively.
Note, this experiment did not include Step 5, use or insemination as does were not available to the researcher.

Table 4 demonstrates the effect of the synergistic continuity on the cumulative data. Sperm that was treated with antioxidants, membrane protectants and bacteriostatic compounds were 7% healthier (improved motility (% motile) and improved membrane and acrosome quality (% membrane % acrosome intact)). This then indicates that these cells would have a greater chance of performing their intended consequence (fertilization of an oocyte) than cells treated with only part of the system, or untreated.

TABLE 4

| | | | % | |
|---|---|---|---|---|
| | | | membrane | % change |
| Holding | Freezing | | & acrosome | from |
| Medium | Medium | % motile | intact | control |
| Control | Control | 35.85 | 56.6 | |
| Control | Treated | 41.025 | 49.5 | −13% |
| Treated | Control | 39.925 | 50.05 | −12% |
| Treated | Treated | 42.875 | 60.55 | 7% |

While the invention has been described in connection with some preferred embodiments, it is not intended to limit the scope of the invention to the particular form set forth, but on the contrary, it is intended to cover such alternatives, modifications, and equivalents as may be included within the spirit and scope of the invention as defined by the statements of inventions. Examples of alternative claims may include:
1. A method of protecting in vitro biological cells with synergistic continuity comprising the steps of:
   harvesting a collection of biological cells from an in vivo source;
   preserving said collection of said biological cells based on an anticipated cell damage limiting regimen and a predetermined use;
   providing a holding media applicable for said anticipated cell damage limiting regimen and said predetermined use, wherein said holding media comprises at least two components selected from an antioxidant, a phospholipase inhibitor, membrane stabilizing agent, and an antimicrobial agent;
   adding said holding media to said collection of said biological cells;
   transporting said collection of said biological cells in said holding media based on said anticipated cell damage limiting regimen and said predetermined use;
   receiving said collection of said biological cells after said step of transporting said collection of said biological cells in said holding media;
   preparing said biological cells to be hypothermically treated;
   hypothermically treating said biological cells;
   warming said biological cells; and
   using said biological cells for said predetermined use.
2. A method of protecting in vitro biological cells as described in clause 1, or any other clause, wherein said collection of biological cells is selected from a group consisting of cells, tissues, sperm, equine sperm, bovine sperm, caprine sperm, ovine sperm, porcine sperm, fowl sperm, ovaries, oocytes, embryos, organs, stem cells, genetically modified cells, artificially derived cells, and any combination thereof.
3. A method of protecting in vitro biological cells as described in clause 1, or any other clause, wherein said in vivo source is selected from a group consisting of mammal, human, rodents, equine, bovine, caprine, ovine, porcine, fowl, fish, shell fish, reptile, nephropidae, poikilothermic, and aquatic vertebrates.
4. A method of protecting in vitro biological cells as described in clause 1, or any other clause, wherein said predetermined use is selected from a group consisting of insemination, implantation, culturing, research, diagnostic testing, replication, gamete preservation, genetic preservation, cryopreservation, reproduction, and any combination thereof.
5. A method of protecting in vitro biological cells as described in clause 1, or any other clause, wherein said holding media comprises at least one additional component selected from a group consisting of natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, or Tetrapleura tetraptera extract, silibinin, phosphofructokinase, carnosine, lignans, fagaronine, ellagitannins, eschscholtzidine, saponin, and any combination thereof.
6. A method of protecting in vitro biological cells as described in clause 5, or any other clause, wherein said plant extract comprises a plant extract derived from a source selected from a group consisting of sap, berries, seeds, leaves, flowers, stems, bark, and any combination thereof.
7. A method of protecting in vitro biological cells as described in clause 5, or any other clause, wherein said plant extract is selected from a group consisting of a crude plant extract, a single source plant extract, a combination of extracts from more than one source, alcohol extracts, juice components, sodium hydroxide extracts, aqueous extracts, hydroglycerine extracts, and any combination thereof.
8. A method of protecting in vitro biological cells as described in clause 1, or any other clause, wherein said holding media comprises an anti-microbial component selected from a group consisting of heptadecanoyl ethanolamide, triterpenes, steroid-like triterpenes, lipoglycopeptides, natural gums, natural resins, essential oils, tea tree oil, hyperenone A, hypercalin B, hyperphorin, phenolics, polyphenols, terpenes, flavonoids, alkaloids, propolis, spermidine, rutin, quercetin, coumarins, kaempferol, stigmasterol, campesterol, tocopherol, carotenoids, horseradish juice extract, tobramycin and any combination thereof.
9. A method of protecting in vitro biological cells as described in clause 1, or any other clause, wherein said phospholipase inhibitor comprises a phospholipase A2 inhibitor.
10. A method of protecting in vitro biological cells as described in clause 1, or any other clause, wherein said phospholipase inhibitor is selected from a group consisting of zinc, manganese, citric acid, and any combination thereof.
11. A method of protecting in vitro biological cells as described in clause 1, or any other clause, wherein said phospholipase inhibitor is selected from a group consisting of a plant extract, cucurmin, Gingko biloba extract, Centella asiatica extract, Hippophae extract, a chemical phospholipase inhibitor, pyrrolidone-based compounds, aristolochic acid, spermine neomycin sulfate, and any combination thereof.
12. A method of protecting in vitro biological cells as described in clause 1, or any other clause, wherein said step of adding said holding media to said collection of said biological cells comprises the step of adding enough holding media to said collection of said biological cells to last throughout said step of transporting said collection of said biological cells.
13. A method of protecting in vitro biological cells as described in clause 1, or any other clause, wherein said step of providing said holding media applicable for said anticipated cell damage limiting regimen and said predetermined use comprises the step of providing time released compounds in said holding media.
14. A method of protecting in vitro biological cells as described in clause 1, or any other clause, and further comprising a step of adding additional holding media to said collection of biological cells during said step of transporting said collection of said biological cells.
15. A method of protecting in vitro biological cells as described in clause 5, or any other clause, wherein said cryoprotectant is selected from a group consisting of glycerol, glycine, dimethylsulfoxide, proline, modified betaines, glycinebetaine, dimethyl sulphoniopropionate, cyclohexanediol, methyl formamide, dimethyl formamide, ethylene glycol, trehalose, concentrated complex sugars, tree sap, concentrated sugars, penetrating cryoprotectants, non-penetrating cryoprotectants, plant extracts, and any combination thereof.
16. A method of protecting in vitro biological cells as described in clause 1, or any other clause, wherein said step of preserving said collection of said biological cells based on said anticipated cell damage limiting regimen and said predetermined use comprises the step of cooling said collection of biological cells.
17. A method of protecting in vitro biological cells as described in clause 1, or any other clause, wherein said step of transporting said collection of said biological cells in said holding media comprises the step of cooling said collection of said biological cells in said holding media.
18. A method of protecting in vitro biological cells as described in clauses 16, 17, or any other clause, wherein said step of cooling said collection of biological cells comprises the step of cooling said collection of biological cells to a temperature selected from a group consisting of between about 0° C. to about 37° C., about 4° C., about 10° C., and about 17° C.
19. A method of protecting in vitro biological cells as described in clauses 16, 17, or any other clause, wherein said step of cooling said collection of biological cells comprises the step of cooling said collection of biological cells at a cooling rate from between about 0.01° C./min to about 1° C./min.
20. A method of protecting in vitro biological cells as described in clause 1, or any other clause, wherein said step of preserving said collection of said biological cells based on said anticipated cell damage limiting regimen and said predetermined use comprises the step of pre-processing said collection of biological cells during said step of transporting said collection of said biological cells based on said anticipated cell damage limiting regimen and said predetermined use.
21. A method of protecting in vitro biological cells as described in clause 1, or any other clause, wherein said step of preserving said collection of said biological cells based on said anticipated cell damage limiting regimen and said predetermined use comprises the step of maintaining in vivo redox potential within said biological cells.
22. A method of protecting in vitro biological cells as described in clause 21, or any other clause, wherein said step of maintaining said in vivo redox potential within said biological cells comprise the step of utilizing a combination of lipid soluble and aqueous antioxidants in said holding media.
23. A method of protecting in vitro biological cells as described in clause 22, or any other clause, wherein said lipid soluble and aqueous antioxidants comprises a plant extract.
24. A method of protecting in vitro biological cells as described in clause 1, or any other clause, wherein said step of preserving said collection of said biological cells based on said anticipated cell damage limiting regimen and said predetermined use comprise the step of utilizing a system selected from a group consisting of microfluidics and flow cytometry.
25. A method of protecting in vitro biological cells as described in clause 1, or any other clause, wherein said step of preserving said collection of said biological cells based on said anticipated cell damage limiting regimen and said predetermined use comprise the step of utilizing a system to create a uniform environment around said biological cells, said system selected from a group consisting of microfluidics, encapsulation, creating liposomes, creating a micelle, creating a biological cage structure, and any combination thereof.
26. A method of protecting in vitro biological cells as described in clause 1, or any other clause, wherein said step of receiving said collection of said biological cells after said step of transporting said biological cells in said holding media comprises the step of providing shipped biological cells with a characteristic selected from a group consisting of reduced bacterial growth, increased bacteriostatic effect, and increased bactericidal effects.
27. A method of protecting in vitro biological cells as described in clause 1, or any other clause, wherein said step of preparing said biological cells to be hypothermically treated comprises the step of adding hypothermic components to said shipped biological cells.
28. A method of protecting in vitro biological cells as described in clause 27, or any other clause, wherein said hypothermic components is selected from a group consisting of antibiotics, natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemical s, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
29. A method of protecting in vitro biological cells as described in clause , or any other clause,1 wherein said step of preparing said biological cells to be hypothermically treated comprises the step of utilizing less antibiotics with said biological cells, wherein said less antibiotics is selected from a group consisting of less than about 50 IU/ml penicillin, less than about 100 IU/ml penicillin, less than about50 μg/ml streptomycin, less than about 100 μg/ml streptomycin, less than about 500 ug/ml streptomycin, less than about 500 IU/ml penicillin, less than about 150 ug/ml lincomycin, and less than about 300 ug/ml spectinomycin.
30. A method of protecting in vitro biological cells as described in clause 1, or any other clause, wherein said step of preparing said biological cells to be hypothermically treated comprises the step of adding antibiotics to said shipped biological cells and substituting at least part of said antibiotics with a plant extract.
31. A method of protecting in vitro biological cells as described in clause 30, or any other clause, wherein said step of substituting at least part of said antibiotics with a plant extract is selected from a group consisting of: substituting about 10% of the antibiotic with a plant extract; substituting about 20% of the antibiotic with a plant extract; substituting about 30% of the antibiotic with a plant extract; substituting about 40% of the antibiotic with a plant extract; substituting about 50% of the antibiotic with a plant extract; substituting about 60% of the antibiotic with a plant extract; substituting about 70% of the antibiotic with a plant extract; substituting about 80% of the antibiotic with a plant extract; substituting about 90% of the antibiotic with a plant extract; and substituting about 100% of the antibiotic with a plant extract.
32. A method of protecting in vitro biological cells as described in clause 1, or any other clause, wherein said step of preparing said biological cells to be hypothermically treated comprises the step of adding an antioxidant to said biological cells.
33. A method of protecting in vitro biological cells as described in clause 32, or any other clause, wherein said antioxidant is selected from a group consisting of allene oxide synthase, phenolics, flavonoids, ascorbic acid, tocopherols, carotenoids, tannins, butylated hydroxyanisole, butylated hydroxytoluene, tert-butylhydroxyquinone, propyl gallate, and compounds, plant derived or synthetic, sufficient to reduce or scavenge reactive oxygen species superoxide, hydroxyl, peroxyl, alkoxyl, nitric oxide, singlet oxygen, hydrogen peroxide, and any combination thereof.
34. A method of protecting in vitro biological cells as described in clause 1, 20, or any other clause, and further comprising the step of reducing an amount of in vitro exposure laboratory time spent on said step of preparing said biological cells to be hypothermically preserved.
35. A method of protecting in vitro biological cells as described in clause 1, or any other clause, wherein said anticipated cell damage limiting regimen comprises a reduction in cell damage, said cell damage caused from an aspect selected from a group consisting of biological contamination, chemical contamination, contamination caused by invasive species, chemical residues, detergents, disinfectant residues, solvent compounds, organic compounds, photo activation, photo modification, improper handling, bacteria, fungi, mycoplasma, virus, and any combination thereof.
36. A method of protecting in vitro biological cells as described in clause 34, or any other clause, wherein said step of reducing said amount of in vitro exposure laboratory time spent on said step of preparing said biological cells to be hypothermically preserved comprise the step of decreasing an equilibration time of said biological cells at said laboratory in preparation for cryopreservation and exposure to an osmotic agent
37. A method of protecting in vitro biological cells as described in clause 28, or any other clause, wherein said osmotic agent comprise a plant extract.
38. A method of protecting in vitro biological cells as described in clause 1, or any other clause, wherein said step of preparing said biological cells to be hypothermically treated and said step of hypothermically treating said biological cells comprises a hypothermic treatment selected from a group consisting of cooling, cryopreservation, freeze-drying, lyophilization, and vitrification.
39. A method of protecting in vitro biological cells as described in clause 1, or any other clause, wherein said step of preparing said biological cells to be hypothermically treated comprises the step of preparing said biological cells to be cryopreserved; wherein said step of hypothermically treating said biological cells comprises the step of cryopreserving said biological cells; and wherein said step of warming said biological cells comprises the step of thawing said biological cells.
40. A method of protecting in vitro biological cells as described in clause 1, or any other clause, and further comprising the step of utilizing a single collection of biological cells for said step of using said biological cells for said predetermined use.
41. A method of protecting in vitro biological cells as described in clause 1, or any other clause, wherein said step of using said biological cells for said predetermined use comprises the step of providing an improved post-warm cellular health.
42. A method of protecting in vitro biological cells as described in clause 41, or any other clause, wherein said improved post-warm cellular health comprises greater than about 25% pregnancy rate artificial insemination of post-warmed bovine sperm cells.
43. A method of protecting in vitro biological cells as described in clause 1, or any other clause, wherein said step of preserving said collection of said biological cells comprises the step of encapsulating said biological cells.
44. A method of protecting in vitro biological cells as described in clause 1, or any other clause, wherein said step of preserving said collection of said biological cells comprise the step of limiting oxygen exposure to said biological cells.
45. A method of protecting in vitro biological cells as described in clause 1, or any other clause, wherein said step of preserving said collection of said biological cells comprises the step of creating a uniform environment around said biological cells.
46. A method of protecting in vitro biological cells as described in clause 45, or any other clause, wherein said step of creating said uniform environment around said biological cells comprises the step of creating a cage-like environment around each of said biological cells.
47. A method of protecting in vitro biological cells as described in clause 46, or any other clause, wherein said step of creating a cage-like environment around each of said biological cells comprises the step of interacting compounds with a phospholipid head group of said biological cells.
48. A method of protecting in vitro biological cells as described in clause 45, or any other clause, wherein said step of creating a uniform environment around said biological cells comprises the step of adding compounds to said collection of biological cells, said compounds selected from a group consisting of membrane lipids, glycolipids, cholesterol, free fatty acids, phosphoglycerides, sterols, sphingolipids, membrane proteins, salts, agarose, and any combination thereof.
49. A method of protecting in vitro biological cells as described in clause 45, or any other clause, wherein said step of creating a uniform environment around said biological cells comprises the step of encapsulating said biological cells in a microenvironment.
50. A method of protecting in vitro biological cells as described in clause 49, or any other clause, wherein said step of encapsulating said biological cells in a microenvironment comprise the step of adding liposomes or micelles to said collection of biological cells.
51. A method of protecting in vitro biological cells as described in clause 49, or any other clause, wherein said step of encapsulating said biological cells in a microenvironment comprise the step of utilizing a microfluidic system.
52. A method of protecting in vitro biological cells as described in clause 49, or any other clause, wherein said microenvironment comprises a component selected form a group consisting of antioxidant, plant lipid, egg yolk, and any combination thereof.
53. A method of protecting in vitro biological cells as described in clause 49, or any other clause, and further comprising the step of surrounding said microenvironment with a media.
54. A method of protecting in vitro biological cells as described in clause 53, or any other clause, wherein said media comprises agarose.
55. A method of protecting in vitro biological cells as described in clause 49, or any other clause, wherein said microenvironment is processed selected from a group consisting of cooling said microenvironment to between about 0° C. to about 37° C., cooling said microenvironment to about 4° C., cooling said microenvironment to about 10° C., cooling said microenvironment to about 17° C., freezing said microenvironment, freezing said microenvironment to about −20° C., and freezing said microenvironment to about −196° C.
56. A method of protecting in vitro biological cells as described in clause 49, or any other clause, and further comprising the step of releasing said microenvironment at 20° C. or up to 37° C.
57. A method of protecting in vitro biological cells as described in clause 43, or any other clause, wherein said step of encapsulating said biological cells comprises a step selected from a group consisting of providing a micellular structure around said biological cells; providing a lipid layer around said biological cells, and providing a lipid monolayer around said biological cells, and providing a lipid bilayer around said biological cells.
58. A method of protecting in vitro biological cells as described in clause 46, or any other clause, wherein said cage-like environment comprises an encapsulation of said biological cells with a three-dimensional complex.
59. A method of protecting in vitro biological cells as described in clause 49, or any other clause, wherein said microenvironment can be achieved by utilizing microfluidics to create said microenvironment.
60. A method of protecting in vitro biological cells as described in clause 46, or any other clause, wherein said cage-like environment comprises compounds selected from a group consisting of lipids, salts, proteins, BSA protein, phosphatidyl serine, agarose, and any combination thereof.
61. A method of protecting in vitro biological cells as described in clause 45, or any other clause, wherein said step of creating said uniform environment around said biological cells comprises the step of adding fatty acids to said collection of biological cells.
62. A method of protecting in vitro biological cells as described in clause 61, or any other clause, wherein said step of adding fatty acids to said collection of biological cells comprises the step of adding from about 0.5% to about 10% v/v of fatty acids to said collection of biological cells.
63. A method of protecting in vitro biological cells as described in clause 45, or any other clause, wherein said step of creating said uniform environment around said biological cells comprises the step of adding lipids containing about 40% linolenic acid (18:3), about 15% linoleic (18:2) and about 20% palmitic to said collection of biological cells.
64. A method of protecting in vitro biological cells as described in clause 45, or any other clause, wherein said step of creating said uniform environment around said biological cells comprises the step of providing lipids and biological cells together encapsulated in a micellular or liposomal structure.
65. A method of protecting in vitro biological cells as described in clause 45, or any other clause, wherein said step of creating said uniform environment around said biological cells comprises the step of adding a blend of lipids, free fatty acids, phospholipids, and cholesterol optimally beneficial to an individual cell type and a cell derivation.
66. A method of protecting in vitro biological cells as described in clause 1, or any other clause, and further comprising the step of providing said holding media in a preservation kit for biological cells.
67. A method of protecting in vitro biological cells as described in clause 66, or any other clause, wherein said preservation kit comprises at least two components selected from an antioxidant, a phospholipase inhibitor, and an antimicrobial agent.
68. A method of protecting in vitro biological cells as described in clauses 5, 22, 28, 48, 60, 64, or any other clause, wherein said lipid is selected from a group consisting of lipids, free fatty acids, phospholipids, proteins, glycoproteins, and lipoproteins.
69. A method of protecting in vitro biological cells with synergistic continuity comprising the steps of:
   harvesting a collection of biological cells from an in vivo source;
   preserving said collection of said biological cells based on an anticipated cell damage limiting regimen and a predetermined use;
   providing a holding media applicable for said anticipated cell damage limiting regimen and said predetermined use;
   adding said holding media to said collection of said biological cells;
   transporting said collection of said biological cells in said holding media based on said anticipated cell damage limiting regimen and said predetermined use;
   receiving said collection of said biological cells after said step of transporting said collection of said biological cells in said holding media;
   preparing said biological cells to be hypothermically treated;
   hypothermically treating said biological cells;
   warming said biological cells; and
   using said biological cells for said predetermined use.
70. A method of protecting in vitro biological cells as described in clause 69, or any other clause, wherein said collection of biological cells is selected from a group consisting of cells, tissues, sperm, equine sperm, bovine sperm, caprine sperm, ovine sperm, porcine sperm, fowl sperm, ovaries, oocytes, embryos, organs, stem cells, genetically modified cells, artificially derived cells, and any combination thereof.
71. A method of protecting in vitro biological cells as described in clause 69, or any other clause, wherein said in vivo source is selected from a group consisting of mammal, human, rodents, equine, bovine, caprine, ovine, porcine, fowl, fish, shell fish, reptile, nephropidae, poikilothermic, and aquatic vertebrates.
72. A method of protecting in vitro biological cells as described in clause 69, or any other clause, wherein said predetermined use is selected from a group consisting of insemination, implantation, culturing, research, diagnostic testing, replication, gamete preservation, genetic preservation, cryopreservation, reproduction, and any combination thereof.
73. A method of protecting in vitro biological cells as described in clause 69, or any other clause, wherein said holding media comprises at least one component selected from a group consisting of natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
74. A method of protecting in vitro biological cells as described in clause 73, or any other clause, wherein said plant extract comprises a plant extract derived from a source selected from a group consisting of sap, berries, seeds, leaves, flowers, stems, bark, and any combination thereof.
75. A method of protecting in vitro biological cells as described in clause 73, or any other clause, wherein said plant extract is selected from a group consisting of a crude plant extract, a single source plant extract, a combination of extracts from more than one source, alcohol extracts, juice components, sodium hydroxide extracts, aqueous extracts, hydroglycerine extracts, and any combination thereof.
76. A method of protecting in vitro biological cells as described in clause 69, or any other clause, wherein said holding media comprises an anti-microbial component selected from a group consisting of heptadecanoyl ethanolamide, triterpenes, steroid-like triterpenes, lipoglycopeptides, natural gums, natural resins, essential oils, tea tree oil, hyperenone A, hypercalin B, hyperphorin, phenolics, polyphenols, terpenes, flavonoids, alkaloids, propolis, spermidine, rutin, quercetin, coumarins, kaempferol, stigmasterol, campesterol, tocopherol, carotenoids, horseradish juice extract, tobramycin and any combination thereof.
77. A method of protecting in vitro biological cells as described in clause 69, or any other clause, wherein said holding media comprises at least two components selected from an antioxidant, a phospholipase inhibitor, membrane stabilizing agent, and an antimicrobial agent.
78. A method of protecting in vitro biological cells as described in clause 73, or any other clause, wherein said phospholipase inhibitor comprises a phospholipase A2 inhibitor.
79. A method of protecting in vitro biological cells as described in clause 73, or any other clause, wherein said phospholipase inhibitor is selected from a group consisting of zinc, manganese, citric acid, and any combination thereof.
80. A method of protecting in vitro biological cells as described in clause 73, or any other clause, wherein said phospholipase inhibitor is selected from a group consisting of a plant extract, cucurmin, Gingko biloba extract, Centella asiatica extract, Hippophae extract, a chemical phospholipase inhibitor, pyrrolidone-based compounds, aristolochic acid, spermine neomycin sulfate, and any combination thereof.
81. A method of protecting in vitro biological cells as described in clause 73, 77, or any other clause, wherein said microbial inhibitor is a plant derived component.
82. A method of protecting in vitro biological cells as described in clause 69, or any other clause, wherein said step of adding said holding media to said collection of said biological cells comprises the step of adding enough holding media to said collection of said biological cells to last throughout said step of transporting said collection of said biological cells.
83. A method of protecting in vitro biological cells as described in clause 69, or any other clause, wherein said step of providing said holding media applicable for said anticipated cell damage limiting regimen and said predetermined use comprises the step of providing time released compounds in said holding media.
84. A method of protecting in vitro biological cells as described in clause 69, or any other clause, and further comprising a step of adding additional holding media to said collection of biological cells during said step of transporting said collection of said biological cells.
85. A method of protecting in vitro biological cells as described in clause 73, or any other clause, wherein said cryoprotectant is selected from a group consisting of glycerol, glycine, dimethylsulfoxide, proline, modified betaines, glycinebetaine, dimethyl sulphoniopropionate, cyclohexanediol, methyl formamide, dimethyl formamide, ethylene glycol, trehalose, concentrated complex sugars, tree sap, concentrated sugars, penetrating cryoprotectants, non-penetrating cryoprotectants, plant extracts, and any combination thereof.
86. A method of protecting in vitro biological cells as described in clause 69, or any other clause, wherein said step of preserving said collection of said biological cells based on said anticipated cell damage limiting regimen and said predetermined use comprises the step of cooling said collection of biological cells.
87. A method of protecting in vitro biological cells as described in clause 69, or any other clause, wherein said step of transporting said collection of said biological cells in said holding media comprises the step of cooling said collection of said biological cells in said holding media.
88. A method of protecting in vitro biological cells as described in clauses 86, 87, or any other clause, wherein said step of cooling said collection of biological cells comprises the step of cooling said collection of biological cells to a temperature selected from a group consisting of between about 0° C. to about 37° C., about 4° C., about 10° C., and about 17° C.
89. A method of protecting in vitro biological cells as described in clauses 86, 87, or any other clause, wherein said step of cooling said collection of biological cells comprises the step of cooling said collection of biological cells at a cooling rate from between about 0.01° C./min to about 1° C./min.
90. A method of protecting in vitro biological cells as described in clause 69, or any other clause, wherein said step of preserving said collection of said biological cells based on said anticipated cell damage limiting regimen and said predetermined use comprises the step of pre-processing said collection of biological cells during said step of transporting said collection of said biological cells based on said anticipated cell damage limiting regimen and said predetermined use.
91. A method of protecting in vitro biological cells as described in clause 69, or any other clause, wherein said step of preserving said collection of said biological cells based on said anticipated cell damage limiting regimen and said predetermined use comprises the step of maintaining in vivo redox potential within said biological cells.
92. A method of protecting in vitro biological cells as described in clause 91, or any other clause, wherein said step of maintaining said in vivo redox potential within said biological cells comprise the step of utilizing a combination of lipid soluble and aqueous antioxidants in said holding media.
93. A method of protecting in vitro biological cells as described in clause 92, or any other clause, wherein said lipid soluble and aqueous antioxidants comprises a plant extract.
94. A method of protecting in vitro biological cells as described in clause 69, or any other clause, wherein said step of preserving said collection of said biological cells based on said anticipated cell damage limiting regimen and said predetermined use comprise the step of utilizing a system selected from a group consisting of microfluidics, and flow cytometry.
95. A method of protecting in vitro biological cells as described in clause 69, or any other clause, wherein said step of preserving said collection of said biological cells based on said anticipated cell damage limiting regimen and said predetermined use comprise the step of utilizing a system to create a uniform environment around said biological cells, said system selected from a group consisting of microfluidics, encapsulation, creating liposomes, creating a micelle, creating a biological cage structure, and any combination thereof.
96. A method of protecting in vitro biological cells as described in clause 69, or any other clause, wherein said step of receiving said collection of said biological cells after said step of transporting said biological cells in said holding media comprises the step of providing shipped biological cells with a characteristic selected from a group consisting of reduced bacterial growth, increased bacteriostatic effect, and increased bactericidal effects.
97. A method of protecting in vitro biological cells as described in clause 69, or any other clause, wherein said step of preparing said biological cells to be hypothermically treated comprises the step of adding hypothermic components to said shipped biological cells.
98. A method of protecting in vitro biological cells as described in clause 97, or any other clause, wherein said hypothermic components is selected from a group consisting of antibiotics, natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemical s, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
99. A method of protecting in vitro biological cells as described in clause 69, or any other clause, wherein said step of preparing said biological cells to be hypothermically treated comprises the step of utilizing less antibiotics with said biological cells, wherein said less antibiotics is selected from a group consisting of less than about 50 IU/ml penicillin, less than about 100 IU/ml penicillin, less than about50 μg/ml streptomycin, less than about 100 μg/ml streptomycin, less than about 500 ug/ml streptomycin, less than about 500 IU/ml penicillin, less than about 150 ug/ml lincomycin, and less than about 300 ug/ml spectinomycin.
100. A method of protecting in vitro biological cells as described in clause 69, or any other clause, wherein said step of preparing said biological cells to be hypothermically treated comprises the step of adding antibiotics to said shipped biological cells and substituting at least part of said antibiotics with a plant extract.
101. A method of protecting in vitro biological cells as described in clause 100, or any other clause, wherein said step of substituting at least part of said antibiotics with a plant extract is selected from a group consisting of: substituting about 10% of the antibiotic with a plant extract; substituting about 20% of the antibiotic with a plant extract; substituting about 30% of the antibiotic with a plant extract; substituting about 40% of the antibiotic with a plant extract; substituting about 50% of the antibiotic with a plant extract; substituting about 60% of the antibiotic with a plant extract; substituting about 70% of the antibiotic with a plant extract; substituting about 80% of the antibiotic with a plant extract; substituting about 90% of the antibiotic with a plant extract; and substituting about 100% of the antibiotic with a plant extract.
102. A method of protecting in vitro biological cells as described in clause 69, or any other clause, wherein said step of preparing said biological cells to be hypothermically treated comprises the step of adding an antioxidant to said biological cells.
103. A method of protecting in vitro biological cells as described in clause 102, or any other clause, wherein said antioxidant is selected from a group consisting of allene oxide synthase, phenolics, flavonoids, ascorbic acid, tocopherols, carotenoids, tannins, butylated hydroxyanisole, butylated hydroxytoluene, tert-butylhydroxyquinone, propyl gallate, and compounds, plant derived or synthetic, sufficient to reduce or scavenge reactive oxygen species superoxide, hydroxyl, peroxyl, alkoxyl, nitric oxide, singlet oxygen, hydrogen peroxide, and any combination thereof.
104. A method of protecting in vitro biological cells as described in clause 69, 14, or any other clause, and further comprising the step of reducing an amount of in vitro exposure laboratory time spent on said step of preparing said biological cells to be hypothermically preserved.
105. A method of protecting in vitro biological cells as described in clause 69, or any other clause, wherein said anticipated cell damage limiting regimen comprises a reduction in cell damage, said cell damage caused from an aspect selected from a group consisting of biological contamination, chemical contamination, contamination caused by invasive species, chemical residues, detergents, disinfectant residues, solvent compounds, organic compounds, photo activation, photo modification, improper handling, bacteria, fungi, mycoplasma, virus, and any combination thereof.
106. A method of protecting in vitro biological cells as described in clause 104, or any other clause, wherein said step of reducing said amount of in vitro exposure laboratory time spent on said step of preparing said biological cells to be hypothermically preserved comprise the step of decreasing an equilibration time of said biological cells at said laboratory in preparation for cryopreservation and exposure to an osmotic agent
107. A method of protecting in vitro biological cells as described in clause 98, or any other clause, wherein said osmotic agent comprise a plant extract.
108. A method of protecting in vitro biological cells as described in clause 69, or any other clause, wherein said step of preparing said biological cells to be hypothermically treated and said step of hypothermically treating said biological cells comprises a hypothermic treatment selected from a group consisting of cooling, cryopreservation, freeze-drying, lyophilization, and vitrification.
109. A method of protecting in vitro biological cells as described in clause 69, or any other clause, wherein said step of preparing said biological cells to be hypothermically treated comprises the step of preparing said biological cells to be cryopreserved; wherein said step of hypothermically treating said biological cells comprises the step of cryopreserving said biological cells; and wherein said step of warming said biological cells comprises the step of thawing said biological cells.
110. A method of protecting in vitro biological cells as described in clause 69, or any other clause, and further comprising the step of utilizing a single collection of biological cells for said step of using said biological cells for said predetermined use.
111. A method of protecting in vitro biological cells as described in clause 69, or any other clause, wherein said step of using said biological cells for said predetermined use comprises the step of providing an improved post-warm cellular health.
112. A method of protecting in vitro biological cells as described in clause 111, or any other clause, wherein said improved post-warm cellular health comprises greater than about 25% pregnancy rate artificial insemination of post-warmed bovine sperm cells.
113. A method of protecting in vitro biological cells as described in clause 69, or any other clause, wherein said step of preserving said collection of said biological cells comprises the step of encapsulating said biological cells.
114. A method of protecting in vitro biological cells as described in clause 69, or any other clause, wherein said step of preserving said collection of said biological cells comprise the step of limiting oxygen exposure to said biological cells.
115. A method of protecting in vitro biological cells as described in clause 69, or any other clause, wherein said step of preserving said collection of said biological cells comprises the step of creating a uniform environment around said biological cells.
116. A method of protecting in vitro biological cells as described in clause 115, or any other clause, wherein said step of creating said uniform environment around said biological cells comprises the step of creating a cage-like environment around each of said biological cells.
117. A method of protecting in vitro biological cells as described in clause 116, or any other clause, wherein said step of creating a cage-like environment around each of said biological cells comprises the step of interacting compounds with a phospholipid head group of said biological cells.
118. A method of protecting in vitro biological cells as described in clause 115, or any other clause, wherein said step of creating a uniform environment around said biological cells comprises the step of adding compounds to said collection of biological cells, said compounds selected from a group consisting of membrane lipids, glycolipids, cholesterol, free fatty acids, phosphoglycerides, sterols, sphingolipids, membrane proteins, salts, agarose, and any combination thereof.
119. A method of protecting in vitro biological cells as described in clause 115, or any other clause, wherein said step of creating a uniform environment around said biological cells comprises the step of encapsulating said biological cells in a microenvironment.
120. A method of protecting in vitro biological cells as described in clause 119, or any other clause, wherein said step of encapsulating said biological cells in a microenvironment comprise the step of adding liposomes or micelles to said collection of biological cells.
121. A method of protecting in vitro biological cells as described in clause 119, or any other clause, wherein said step of encapsulating said biological cells in a microenvironment comprise the step of utilizing a microfluidic system.
122. A method of protecting in vitro biological cells as described in clause 119, or any other clause, wherein said microenvironment comprises a component selected form a group consisting of antioxidant, plant lipid, egg yolk, and any combination thereof.
123. A method of protecting in vitro biological cells as described in clause 119, or any other clause, and further comprising the step of surrounding said microenvironment with a media.
124. A method of protecting in vitro biological cells as described in clause 123, or any other clause, wherein said media comprises agarose.
125. A method of protecting in vitro biological cells as described in clause 119, or any other clause, wherein said microenvironment is processed selected from a group consisting of cooling said microenvironment to between about 0° C. to about 37° C., cooling said microenvironment to about 4° C., cooling said microenvironment to about 10° C., cooling said microenvironment to about 17° C., freezing said microenvironment, freezing said microenvironment to about −20° C., and freezing said microenvironment to about −196° C.
126. A method of protecting in vitro biological cells as described in clause 119, or any other clause, and further comprising the step of releasing said microenvironment at 20° C. or up to 37° C.
127. A method of protecting in vitro biological cells as described in clause 113, or any other clause, wherein said step of encapsulating said biological cells comprises a step selected from a group consisting of providing a micellular structure around said biological cells; providing a lipid layer around said biological cells, providing a lipid monolayer around said biological cells, and providing a lipid bilayer around said biological cells.
128. A method of protecting in vitro biological cells as described in clause 116, or any other clause, wherein said cage-like environment comprises an encapsulation of said biological cells with a three-dimensional complex.
129. A method of protecting in vitro biological cells as described in clause 119, or any other clause, wherein said microenvironment can be achieved by utilizing microfluidics to create said microenvironment.
130. A method of protecting in vitro biological cells as described in clause 116, or any other clause, wherein said cage-like environment comprises compounds selected from a group consisting of lipids, salts, proteins, BSA protein, phosphatidyl serine, agarose, and any combination thereof.
131. A method of protecting in vitro biological cells as described in clause 115, or any other clause, wherein said step of creating said uniform environment around said biological cells comprises the step of adding fatty acids to said collection of biological cells.
132. A method of protecting in vitro biological cells as described in clause 131, or any other clause, wherein said step of adding fatty acids to said collection of biological cells comprises the step of adding from about 0.5% to about 10% v/v of fatty acids to said collection of biological cells.
133. A method of protecting in vitro biological cells as described in clause 115, or any other clause, wherein said step of creating said uniform environment around said biological cells comprises the step of adding lipids containing about 40% linolenic acid (18:3), about 15% linoleic (18:2) and about 20% palmitic to said collection of biological cells.
134. A method of protecting in vitro biological cells as described in clause 115, or any other clause, wherein said step of creating said uniform environment around said biological cells comprises the step of providing lipids and biological cells together encapsulated in a micellular or liposomal structure.
135. A method of protecting in vitro biological cells as described in clause 115, or any other clause, wherein said step of creating said uniform environment around said biological cells comprises the step of adding a blend of lipids, free fatty acids, phospholipids, and cholesterol optimally beneficial to an individual cell type and a cell derivation.
136. A method of protecting in vitro biological cells as described in clause 69, or any other clause, and further comprising the step of providing said holding media in a preservation kit for biological cells.
137. A method of protecting in vitro biological cells as described in clause 136, or any other clause, wherein said preservation kit comprises at least two components selected from an antioxidant, a phospholipase inhibitor, and an antimicrobial agent.
138. A method of protecting in vitro biological cells as described in clauses 73, 92, 98, 118, 130, 134, or any other clause, wherein said lipid is selected from a group consisting of lipids, free fatty acids, phospholipids, proteins, glycoproteins, and lipoproteins.
139. A method for maximizing viability of each cell in a collection of biological cells comprising the steps of:
   harvesting a collection of biological cells from an in vivo source;
   establishing a uniform environment around each biological cell of said collection of biological cells; and
   adding a phospholipase inhibitor to said collection of biological cells.
140. A method for maximizing viability of each cell in a collection of biological cells as described in clause 139 wherein said collection of biological cells is selected from a group consisting of cells, tissues, sperm, equine sperm, bovine sperm, caprine sperm, ovine sperm, porcine sperm, fowl sperm, ovaries, oocytes, embryos, organs, stem cells, genetically modified cells, artificially derived cells, and any combination thereof.
141. A method for maximizing viability of each cell in a collection of biological cells as described in clause 139, or any other clause, wherein said in vivo source is selected from a group consisting of mammal, human, rodents, equine, bovine, caprine, ovine, porcine, fowl, fish, shell fish, reptile, nephropidae, poikilothermic, and aquatic vertebrates.
142. A method for maximizing viability of each cell in a collection of biological cells as described in clause 139 , or any other clause, and further comprising the steps of preserving said collection of biological cells based on an anticipated cell damage limiting regimen and a predetermined use; providing a holding media applicable for said anticipated cell damage limiting regimen and said predetermined use; adding said holding media to said collection of said biological cells; transporting said collection of said biological cells in said holding media based on said anticipated cell damage limiting regimen and said predetermined use.
143. A method for maximizing viability of each cell in a collection of biological cells as described in clause 142, or any other clause, wherein said predetermined use is selected from a group consisting of insemination, implantation, culturing, research, diagnostic testing, replication, gamete preservation, genetic preservation, cryopreservation, reproduction, and any combination thereof.
144. A method for maximizing viability of each cell in a collection of biological cells as described in clause 142, or any other clause, wherein said holding media comprises at least one component selected from a group consisting of natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
145. A method for maximizing viability of each cell in a collection of biological cells as described in clause 144, or any other clause, wherein said plant extract comprises a plant extract derived from a source selected from a group consisting of sap, berries, seeds, leaves, flowers, stems, bark, and any combination thereof.
146. A method for maximizing viability of each cell in a collection of biological cells as described in clause 144, or any other clause, wherein said plant extract is selected from a group consisting of a crude plant extract, a single source plant extract, a combination of extracts from more than one source, alcohol extracts, juice components, sodium hydroxide extracts, aqueous extracts, hydroglycerine extracts, and any combination thereof.
147. A method for maximizing viability of each cell in a collection of biological cells as described in clause 142, or any other clause, wherein said holding media comprises an anti-microbial component selected from a group consisting of heptadecanoyl ethanolamide, triterpenes, steroid-like triterpenes, lipoglycopeptides, natural gums, natural resins, essential oils, tea tree oil, hyperenone A, hypercalin B, hyperphorin, phenolics, polyphenols, terpenes, flavonoids, alkaloids, propolis, spermidine, rutin, quercetin, coumarins, kaempferol, stigmasterol, campesterol, tocopherol, carotenoids, horseradish juice extract, tobramycin and any combination thereof.
148. A method for maximizing viability of each cell in a collection of biological cells as described in clause 142, or any other clause, wherein said holding media comprises at least two components selected from an antioxidant, a phospholipase inhibitor, membrane stabilizing agent, and an antimicrobial agent.
149. A method for maximizing viability of each cell in a collection of biological cells as described in clause 139, or any other clause, wherein said phospholipase inhibitor comprises a phospholipase A2 inhibitor.
150. A method for maximizing viability of each cell in a collection of biological cells as described in clause 139, or any other clause, wherein said phospholipase inhibitor is selected from a group consisting of zinc, manganese, citric acid, and any combination thereof.
151. A method for maximizing viability of each cell in a collection of biological cells as described in clause 139, or any other clause, wherein said phospholipase inhibitor is selected from a group consisting of a plant extract, cucurmin, Gingko biloba extract, Centella asiatica extract, Hippophae extract, a chemical phospholipase inhibitor, pyrrolidone-based compounds, aristolochic acid, spermine neomycin sulfate, and any combination thereof.
152. A method for maximizing viability of each cell in a collection of biological cells as described in clauses 144, 148, or any other clause, wherein said microbial inhibitor is a plant derived component.
153. A method for maximizing viability of each cell in a collection of biological cells as described in clause 142, or any other clause, wherein said step of adding said holding media to said collection of said biological cells comprises the step of adding enough holding media to said collection of said biological cells to last throughout said step of transporting said collection of said biological cells.
154. A method for maximizing viability of each cell in a collection of biological cells as described in clause 142, or any other clause, wherein said step of providing said holding media applicable for said predetermined use comprises the step of providing time released compounds in said holding media.
155. A method for maximizing viability of each cell in a collection of biological cells as described in clause 142, or any other clause, and further comprising a step of adding additional holding media to said collection of biological cells during said step of transporting said collection of said biological cells.
156. A method for maximizing viability of each cell in a collection of biological cells as described in clause 144, or any other clause, wherein said cryoprotectant is selected from a group consisting of glycerol, glycine, dimethylsulfoxide, proline, modified betaines, glycinebetaine, dimethyl sulphoniopropionate, cyclohexanediol, methyl formamide, dimethyl formamide, ethylene glycol, trehalose, concentrated complex sugars, tree sap, concentrated sugars, penetrating cryoprotectants, non-penetrating cryoprotectants, plant extracts, and any combination thereof.
157. A method for maximizing viability of each cell in a collection of biological cells as described in clause 139, or any other clause, and further comprising the step of cooling said collection of biological cells.
158. A method for maximizing viability of each cell in a collection of biological cells as described in clause 157, or any other clause, wherein said step of cooling said collection of biological cells comprises the step of cooling said collection of biological cells to a temperature selected from a group consisting of between about 0° C. to about 37° C., about 4° C., about 10° C., and about 17° C.
159. A method for maximizing viability of each cell in a collection of biological cells as described in clause 157, or any other clause, wherein said step of cooling said collection of biological cells comprises the step of cooling said collection of biological cells at a cooling rate from between about 0.01° C./min to about 1° C./min.
160. A method for maximizing viability of each cell in a collection of biological cells as described in clause 142, or any other clause, wherein said step of preserving said collection of said biological cells based on said predetermined use comprises the step of pre-processing said collection of biological cells during said step of transporting said collection of said biological cells based on said predetermined use.
161. A method for maximizing viability of each cell in a collection of biological cells as described in clause 142, or any other clause, wherein said step of preserving said collection of said biological cells based on said predetermined use comprises the step of maintaining in vivo redox potential within said biological cells.
162. A method for maximizing viability of each cell in a collection of biological cells as described in clause 161, or any other clause, wherein said step of maintaining said in vivo redox potential within said biological cells comprise the step of utilizing a combination of lipid soluble and aqueous antioxidants in said holding media.
163. A method for maximizing viability of each cell in a collection of biological cells as described in clause 162, or any other clause, wherein said lipid soluble and aqueous antioxidants comprises a plant extract.
164. A method for maximizing viability of each cell in a collection of biological cells as described in clause 142, or any other clause, wherein said step of preserving said collection of said biological cells based on said predetermined use comprise the step of utilizing a system selected from a group consisting of microfluidics, and flow cytometry.
165. A method for maximizing viability of each cell in a collection of biological cells as described in clause 142, or any other clause, wherein said step of preserving said collection of said biological cells based on said predetermined use comprise the step of utilizing a system to create a uniform environment around said biological cells, said system selected from a group consisting of microfluidics, encapsulation, creating liposomes, creating a micelle, creating a biological cage structure, and any combination thereof.
166. A method for maximizing viability of each cell in a collection of biological cells as described in clause 142, or any other clause, and further comprising the step of receiving said collection of said biological cells after said step of transporting said biological cells in said holding media, wherein said shipped biological cells comprise a characteristic selected from a group consisting of reduced bacterial growth, increased bacteriostatic effect, and increased bactericidal effects.
167. A method for maximizing viability of each cell in a collection of biological cells as described in clause 142, or any other clause, and further comprising the step of adding hypothermic components to said shipped biological cells.
168. A method for maximizing viability of each cell in a collection of biological cells as described in clause 167, or any other clause, wherein said hypothermic components is selected from a group consisting of antibiotics, natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
169. A method for maximizing viability of each cell in a collection of biological cells as described in clause 139 , or any other clause, and further comprising the step of preparing said biological cells to be hypothermically treated by utilizing less antibiotics with said biological cells, wherein said less antibiotics is selected from a group consisting of less than about 50 IU/ml penicillin, less than about 100 IU/ml penicillin, less than about50 μg/ml streptomycin, less than about 100 μg/ml streptomycin, less than about 500 ug/ml streptomycin, less than about 500 IU/ml penicillin, less than about 150 ug/ml lincomycin, and less than about 300 ug/ml spectinomycin.
170. A method for maximizing viability of each cell in a collection of biological cells as described in clause 139, or any other clause, and further comprising the step of preparing said biological cells to be hypothermically treated by adding antibiotics to said shipped biological cells and substituting at least part of said antibiotics with a plant extract.
171. A method for maximizing viability of each cell in a collection of biological cells as described in clause 170, or any other clause, wherein said step of substituting at least part of said antibiotics with a plant extract is selected from a group consisting of: substituting about 10% of the antibiotic with a plant extract; substituting about 20% of the antibiotic with a plant extract; substituting about 30% of the antibiotic with a plant extract; substituting about 40% of the antibiotic with a plant extract; substituting about 50% of the antibiotic with a plant extract; substituting about 60% of the antibiotic with a plant extract; substituting about 70% of the antibiotic with a plant extract; substituting about 80% of the antibiotic with a plant extract; substituting about 90% of the antibiotic with a plant extract; and substituting about 100% of the antibiotic with a plant extract.
172. A method for maximizing viability of each cell in a collection of biological cells as described in clause 139, or any other clause, and further comprising the step of preparing said biological cells to be hypothermically treated by adding an antioxidant to said biological cells.
173. A method for maximizing viability of each cell in a collection of biological cells as described in clause 172, or any other clause, wherein said antioxidant is selected from a group consisting of allene oxide synthase, phenolics, flavonoids, ascorbic acid, tocopherols, carotenoids, tannins, butylated hydroxyanisole, butylated hydroxytoluene, tert-butylhydroxyquinone, propyl gallate, and compounds, plant derived or synthetic, sufficient to reduce or scavenge reactive oxygen species superoxide, hydroxyl, peroxyl, alkoxyl, nitric oxide, singlet oxygen, hydrogen peroxide, and any combination thereof.
174. A method for maximizing viability of each cell in a collection of biological cells as described in clauses 142, 160, or any other clause, and further comprising the step of reducing an amount of in vitro exposure laboratory time spent on preparing said biological cells to be hypothermically preserved.
175. A method for maximizing viability of each cell in a collection of biological cells as described in clause 142, or any other clause, wherein said anticipated cell damage limiting regimen comprises a reduction in cell damage, said cell damage caused from an aspect selected from a group consisting of biological contamination, chemical contamination, contamination caused by invasive species, chemical residues, detergents, disinfectant residues, solvent compounds, organic compounds, photo activation, photo modification, improper handling, bacteria, fungi, mycoplasma, virus, and any combination thereof.
176. A method for maximizing viability of each cell in a collection of biological cells as described in clause 174, or any other clause, wherein said step of reducing said amount of in vitro exposure laboratory time spent on said step of preparing said biological cells to be hypothermically preserved comprise the step of decreasing an equilibration time of said biological cells at said laboratory in preparation for cryopreservation and exposure to an osmotic agent
177. A method for maximizing viability of each cell in a collection of biological cells as described in clause 168, or any other clause, wherein said osmotic agent comprise a plant extract.
178. A method for maximizing viability of each cell in a collection of biological cells as described in clause 139, or any other clause, and further comprising the step of preparing said biological cells to be hypothermically treated with a hypothermic treatment selected from a group consisting of cooling, cryopreservation, freeze-drying, lyophilization, and vitrification.
179. A method for maximizing viability of each cell in a collection of biological cells as described in clause 139, or any other clause, and further comprising the step of utilizing a single collection of biological cells.
180. A method for maximizing viability of each cell in a collection of biological cells as described in clause 142 , or any other clause, and further comprising the steps of receiving said collection of said biological cells after said step of transporting said collection of said biological cells in said holding media; preparing said biological cells to be hypothermically treated; hypothermically treating said biological cells; warming said biological cells; and using said biological cells for said predetermined use.
181. A method for maximizing viability of each cell in a collection of biological cells as described in clause 180, or any other clause, wherein said step of using said biological cells for said predetermined use comprises the step of providing an improved post-warm cellular health.
182. A method for maximizing viability of each cell in a collection of biological cells as described in clause 181, or any other clause, wherein said improved post-warm cellular health comprises greater than about 25% pregnancy rate artificial insemination of post-warmed bovine sperm cells.
183. A method for maximizing viability of each cell in a collection of biological cells as described in clause 142, or any other clause, wherein said step of preserving said collection of said biological cells comprises the step of encapsulating said biological cells.
184. A method for maximizing viability of each cell in a collection of biological cells as described in clause 142, or any other clause, wherein said step of preserving said collection of said biological cells comprise the step of limiting oxygen exposure to said biological cells.
185. A method for maximizing viability of each cell in a collection of biological cells as described in clause 142, or any other clause, wherein said step of preserving said collection of said biological cells comprises the step of creating a uniform environment around said biological cells.
186. A method for maximizing viability of each cell in a collection of biological cells as described in clause 185, or any other clause, wherein said step of creating said uniform environment around said biological cells comprises the step of creating a cage-like environment around each of said biological cells.
187. A method for maximizing viability of each cell in a collection of biological cells as described in clause 186, or any other clause, wherein said step of creating a cage-like environment around each of said biological cells comprises the step of interacting compounds with a phospholipid head group of said biological cells.
188. A method for maximizing viability of each cell in a collection of biological cells as described in clause 185, or any other clause, wherein said step of creating a uniform environment around said biological cells comprises the step of adding compounds to said collection of biological cells, said compounds selected from a group consisting of membrane lipids, glycolipids, cholesterol, free fatty acids, phosphoglycerides, sterols, sphingolipids, membrane proteins, salts, and any combination thereof.
189. A method for maximizing viability of each cell in a collection of biological cells as described in clause 185, or any other clause, wherein said step of creating a uniform environment around said biological cells comprises the step of encapsulating said biological cells in a microenvironment.
190. A method for maximizing viability of each cell in a collection of biological cells as described in clause 189, or any other clause, wherein said step of encapsulating said biological cells in a microenvironment comprise the step of adding liposomes or micelles to said collection of biological cells.
191. A method for maximizing viability of each cell in a collection of biological cells as described in clause 189, or any other clause, wherein said step of encapsulating said biological cells in a microenvironment comprise the step of utilizing a microfluidic system.
192. A method for maximizing viability of each cell in a collection of biological cells as described in clause 189 wherein said microenvironment comprises a component selected form a group consisting of antioxidant, plant lipid, egg yolk, and any combination thereof.
193. A method for maximizing viability of each cell in a collection of biological cells as described in clause 189, or any other clause, and further comprising the step of surrounding said microenvironment with a media.
194. A method for maximizing viability of each cell in a collection of biological cells as described in clause 193, or any other clause, wherein said media comprises agarose.
195. A method for maximizing viability of each cell in a collection of biological cells as described in clause 189, or any other clause, wherein said microenvironment is processed selected from a group consisting of cooling said microenvironment to between about 0° C. to about 37° C., cooling said microenvironment to about 4° C., cooling said microenvironment to about 10° C., cooling said microenvironment to about 17° C., freezing said microenvironment, freezing said microenvironment to about −20° C., and freezing said microenvironment to about -196° C.
196. A method for maximizing viability of each cell in a collection of biological cells as described in clause 189, or any other clause, and further comprising the step of releasing said microenvironment at 20° C. or up to 37° C.
197. A method for maximizing viability of each cell in a collection of biological cells as described in clause 183, or any other clause, wherein said step of encapsulating said biological cells comprises a step selected from a group consisting of providing a micellular structure around said biological cells; providing a lipid layer around said biological cells, providing a lipid monolayer around said biological cells, and providing a lipid bilayer around said biological cells.
198. A method for maximizing viability of each cell in a collection of biological cells as described in clause 186, or any other clause, wherein said cage-like environment comprises an encapsulation of said biological cells with a three-dimensional complex.
199. A method for maximizing viability of each cell in a collection of biological cells as described in clause 189, or any other clause, wherein said microenvironment can be achieved by utilizing microfluidics to create said microenvironment.
200. A method for maximizing viability of each cell in a collection of biological cells as described in clause 186, or any other clause, wherein said cage-like environment comprises compounds selected from a group consisting of lipids, salts, proteins, BSA protein, phosphatidyl serine, agarose, and any combination thereof.
201. A method for maximizing viability of each cell in a collection of biological cells as described in clause 185, or any other clause, wherein said step of creating said uniform environment around said biological cells comprises the step of adding fatty acids to said collection of biological cells.
202. A method for maximizing viability of each cell in a collection of biological cells as described in clause 201, or any other clause, wherein said step of adding fatty acids to said collection of biological cells comprises the step of adding from about 0.5% to about 10% v/v of fatty acids to said collection of biological cells.
203. A method for maximizing viability of each cell in a collection of biological cells as described in clause 185, or any other clause, wherein said step of creating said uniform environment around said biological cells comprises the step of adding lipids containing about 40% linolenic acid (18:3), about 15% linoleic (18:2) and about 20% palmitic to said collection of biological cells.
204. A method for maximizing viability of each cell in a collection of biological cells as described in clause 185, or any other clause, wherein said step of creating said uniform environment around said biological cells comprises the step of providing lipids and biological cells together encapsulated in a micellular or liposomal structure.
205. A method for maximizing viability of each cell in a collection of biological cells as described in clause 185, or any other clause, wherein said step of creating said uniform environment around said biological cells comprises the step of adding a blend of lipids, free fatty acids, phospholipids, and cholesterol optimally beneficial to an individual cell type and a cell derivation.
206. A method for maximizing viability of each cell in a collection of biological cells as described in clause 142, or any other clause, and further comprising the step of providing said holding media in a preservation kit for biological cells.
207. A method for maximizing viability of each cell in a collection of biological cells as described in clause 206, or any other clause, wherein said preservation kit comprises at least two components selected from an antioxidant, a phospholipase inhibitor, and an antimicrobial agent.
208. A method for maximizing viability of each cell in a collection of biological cells as described in clauses 144, 162, 168, 188, 200, 204, or any other clause, wherein said lipid is selected from a group consisting of lipids, free fatty acids, phospholipids, proteins, glycoproteins, and lipoproteins.
209. A method for preserving harvested biological cells comprising the steps of:
   harvesting a collection of biological cells from an in vivo source;
   creating a uniform environment around substantially each biological cell in said collection of said biological cells;
   hypothermically treating said biological cells;
   warming said biological cells; and
   using said biological cells.
210. A method for preserving harvested biological cells as described in clause 209, or any other clause, wherein said collection of biological cells is selected from a group consisting of cells, tissues, sperm, equine sperm, bovine sperm, caprine sperm, ovine sperm, porcine sperm, fowl sperm, ovaries, oocytes, embryos, organs, stem cells, genetically modified cells, artificially derived cells, and any combination thereof.
211. A method for preserving harvested biological cells as described in clause 209, or any other clause, wherein said in vivo source is selected from a group consisting of mammal, human, rodents, equine, bovine, caprine, ovine, porcine, fowl, fish, shell fish, reptile, nephropidae, poikilothermic, and aquatic vertebrates.
212. A method for preserving harvested biological cells as described in clause 209, or any other clause, and further comprising the steps of preserving said collection of said biological cells based on an anticipated cell damage limiting regimen and a predetermined use.
213. A method for preserving harvested biological cells as described in clause 212, or any other clause, wherein said predetermined use is selected from a group consisting of insemination, implantation, culturing, research, diagnostic testing, replication, gamete preservation, genetic preservation, cryopreservation, reproduction, and any combination thereof.
214. A method for preserving harvested biological cells as described in clause 212, or any other clause, and further comprising the steps of providing a holding media applicable for said anticipated cell damage limiting regimen and said predetermined use; and adding said holding media to said collection of said biological cells
215. A method for preserving harvested biological cells as described in clause 214, or any other clause, wherein said holding media comprises at least one component selected from a group consisting of natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
216. A method for preserving harvested biological cells as described in clause 215, or any other clause, wherein said plant extract comprises a plant extract derived from a source selected from a group consisting of sap, berries, seeds, leaves, flowers, stems, bark, and any combination thereof.
217. A method for preserving harvested biological cells as described in clause 215, or any other clause, wherein said plant extract is selected from a group consisting of a crude plant extract, a single source plant extract, a combination of extracts from more than one source, alcohol extracts, juice components, sodium hydroxide extracts, aqueous extracts, hydroglycerine extracts, and any combination thereof.
218. A method for preserving harvested biological cells as described in clause 214, or any other clause, wherein said holding media comprises an anti-microbial component selected from a group consisting of heptadecanoyl ethanolamide, triterpenes, steroid-like triterpenes, lipoglycopeptides, natural gums, natural resins, essential oils, tea tree oil, hyperenone A, hypercalin B, hyperphorin, phenolics, polyphenols, terpenes, flavonoids, alkaloids, propolis, spermidine, rutin, quercetin, coumarins, kaempferol, stigmasterol, campesterol, tocopherol, carotenoids, horseradish juice extract, tobramycin and any combination thereof.
219. A method for preserving harvested biological cells as described in clause 214, or any other clause, wherein said holding media comprises at least two components selected from an antioxidant, a phospholipase inhibitor, membrane stabilizing agent, and an antimicrobial agent.
220. A method for preserving harvested biological cells as described in clause 215, or any other clause, wherein said phospholipase inhibitor comprises a phospholipase A2 inhibitor.
221. A method for preserving harvested biological cells as described in clause 215, or any other clause, wherein said phospholipase inhibitor is selected from a group consisting of zinc, manganese, citric acid, and any combination thereof.
222. A method for preserving harvested biological cells as described in clause 215, or any other clause, wherein said phospholipase inhibitor is selected from a group consisting of a plant extract, cucurmin, Gingko biloba extract, Centella asiatica extract, Hippophae extract, a chemical phospholipase inhibitor, pyrrolidone-based compounds, aristolochic acid, spermine neomycin sulfate, and any combination thereof.
223. A method for preserving harvested biological cells as described in clauses 215, 219, or any other clause, wherein said microbial inhibitor is a plant derived component.
224. A method for preserving harvested biological cells as described in clause 214, or any other clause, wherein said step of adding said holding media to said collection of said biological cells comprises the step of adding enough holding media to said collection of said biological cells to last throughout a step of transporting said collection of said biological cells.
225. A method for preserving harvested biological cells as described in clause 214, or any other clause, wherein said step of providing said holding media applicable for said predetermined use comprises the step of providing time released compounds in said holding media.
226. A method for preserving harvested biological cells as described in clause 214, or any other clause, and further comprising a step of adding additional holding media to said collection of biological cells during a step of transporting said collection of said biological cells.
227. A method for preserving harvested biological cells as described in clause 215, or any other clause, wherein said cryoprotectant is selected from a group consisting of glycerol, glycine, dimethylsulfoxide, proline, modified betaines, glycinebetaine, dimethyl sulphoniopropionate, cyclohexanediol, methyl formamide, dimethyl formamide, ethylene glycol, trehalose, concentrated complex sugars, tree sap, concentrated sugars, penetrating cryoprotectants, non-penetrating cryoprotectants, plant extracts, and any combination thereof.
228. A method for preserving harvested biological cells as described in clause 212, or any other clause, wherein said step of preserving said collection of said biological cells based on said predetermined use comprises the step of cooling said collection of biological cells.
229. A method for preserving harvested biological cells as described in clause 209, or any other clause, and further comprising the step of transporting said collection of said biological cells and cooling said collection of said biological cells during said transporting step.
230. A method for preserving harvested biological cells as described in clauses 228, 229, or any other clause, wherein said step of cooling said collection of biological cells comprises the step of cooling said collection of biological cells to a temperature selected from a group consisting of between about 0° C. to about 37° C., about 4° C., about 10° C., and about 17° C.
231. A method for preserving harvested biological cells as described in clauses 228, 229, or any other clause, wherein said step of cooling said collection of biological cells comprises the step of cooling said collection of biological cells at a cooling rate from between about 0.01° C./min to about 1° C./min.
232. A method for preserving harvested biological cells as described in clause 212, or any other clause, wherein said step of preserving said collection of said biological cells based on said predetermined use comprises the step of pre-processing said collection of biological cells during a step of transporting said collection of said biological cells based on said predetermined use.
233. A method for preserving harvested biological cells as described in clause 212, or any other clause, wherein said step of preserving said collection of said biological cells based on said predetermined use comprises the step of maintaining in vivo redox potential within said biological cells.
234. A method for preserving harvested biological cells as described in clause 233, or any other clause, wherein said step of maintaining said in vivo redox potential within said biological cells comprise the step of utilizing a combination of lipid soluble and aqueous antioxidants in said holding media.
235. A method for preserving harvested biological cells as described in clause 234, or any other clause, wherein said lipid soluble and aqueous antioxidants comprises a plant extract.
236. A method for preserving harvested biological cells as described in clause 212, or any other clause, wherein said step of preserving said collection of said biological cells based on said predetermined use comprise the step of utilizing a system selected from a group consisting of microfluidics, and flow cytometry.
237. A method for preserving harvested biological cells as described in clause 209, or any other clause, wherein said uniform environment around said biological cells is creating by a system selected from a group consisting of microfluidics, encapsulation, creating liposomes, creating a micelle, creating a biological cage structure, and any combination thereof.
238. A method for preserving harvested biological cells as described in clause 209, or any other clause, and further comprising the steps of transporting said biological cells comprises the step of providing shipped biological cells and receiving said biological cells after said step of transporting said biological cells, wherein said shipped biological cells comprise a characteristic selected from a group consisting of reduced bacterial growth, increased bacteriostatic effect, and increased bactericidal effects.
239. A method for preserving harvested biological cells as described in clause 209, or any other clause, wherein said step of preparing said biological cells to be hypothermically treated comprises the step of adding hypothermic components to said shipped biological cells.
240. A method for preserving harvested biological cells as described in clause 239, or any other clause, wherein said hypothermic components is selected from a group consisting of antibiotics, natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemical s, carbohydrates, phenolics, polyphenol, organic acids, lipid, sugar, salt, protein, compound molecules, phytochemicals, secondary metabolites of plants, plant extract, sea buckthorn extract, Fagara Zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
241. A method for preserving harvested biological cells as described in clause 209, or any other clause, wherein said step of preparing said biological cells to be hypothermically treated comprises the step of utilizing less antibiotics with said biological cells, wherein said less antibiotics is selected from a group consisting of less than about 50 IU/ml penicillin, less than about 100 IU/ml penicillin, less than about50 μg/ml streptomycin, less than about 100 μg/ml streptomycin, less than about 500 ug/ml streptomycin, less than about 500 IU/ml penicillin, less than about 150 ug/ml lincomycin, and less than about 300 ug/ml spectinomycin.
242. A method for preserving harvested biological cells as described in clause 209, or any other clause, wherein said step of preparing said biological cells to be hypothermically treated comprises the step of adding antibiotics to said shipped biological cells and substituting at least part of said antibiotics with a plant extract.
243. A method for preserving harvested biological cells as described in clause 242, or any other clause, wherein said step of substituting at least part of said antibiotics with a plant extract is selected from a group consisting of: substituting about 10% of the antibiotic with a plant extract; substituting about 20% of the antibiotic with a plant extract; substituting about 30% of the antibiotic with a plant extract; substituting about 40% of the antibiotic with a plant extract; substituting about 50% of the antibiotic with a plant extract; substituting about 60% of the antibiotic with a plant extract; substituting about 70% of the antibiotic with a plant extract; substituting about 80% of the antibiotic with a plant extract; substituting about 90% of the antibiotic with a plant extract; and substituting about 100% of the antibiotic with a plant extract.
244. A method for preserving harvested biological cells as described in clause 209, or any other clause, wherein said step of preparing said biological cells to be hypothermically treated comprises the step of adding an antioxidant to said biological cells.
245. A method for preserving harvested biological cells as described in clause 244, or any other clause, wherein said antioxidant is selected from a group consisting of allene oxide synthase, phenolics, flavonoids, ascorbic acid, tocopherols, carotenoids, tannins, butylated hydroxyanisole, butylated hydroxytoluene, tert-butylhydroxyquinone, propyl gallate, and compounds, plant derived or synthetic, sufficient to reduce or scavenge reactive oxygen species superoxide, hydroxyl, peroxyl, alkoxyl, nitric oxide, singlet oxygen, hydrogen peroxide, and any combination thereof.
246. A method for preserving harvested biological cells as described in clauses 209, 232, or any other clause, and further comprising the step of reducing an amount of in vitro exposure laboratory time spent on said step of preparing said biological cells to be hypothermically preserved.
247. A method for preserving harvested biological cells as described in clause 212, or any other clause, wherein said anticipated cell damage limiting regimen comprises a reduction in cell damage, said cell damage caused from an aspect selected from a group consisting of biological contamination, chemical contamination, contamination caused by invasive species, chemical residues, detergents, disinfectant residues, solvent compounds, organic compounds, photo activation, photo modification, improper handling, bacteria, fungi, mycoplasma, virus, and any combination thereof.
248. A method for preserving harvested biological cells as described in clause 246, or any other clause, wherein said step of reducing said amount of in vitro exposure laboratory time spent on said step of preparing said biological cells to be hypothermically preserved comprise the step of decreasing an equilibration time of said biological cells at said laboratory in preparation for cryopreservation and exposure to an osmotic agent
249. A method for preserving harvested biological cells as described in clause 240, or any other clause, wherein said osmotic agent comprise a plant extract.
250. A method for preserving harvested biological cells as described in clause 209, or any other clause, wherein said step of preparing said biological cells to be hypothermically treated and said step of hypothermically treating said biological cells comprises a hypothermic treatment selected from a group consisting of cooling, cryopreservation, freeze-drying, lyophilization, and vitrification.
251. A method for preserving harvested biological cells as described in clause 209, or any other clause, wherein said step of preparing said biological cells to be hypothermically treated comprises the step of preparing said biological cells to be cryopreserved; wherein said step of hypothermically treating said biological cells comprises the step of cryopreserving said biological cells; and wherein said step of warming said biological cells comprises the step of thawing said biological cells.
252. A method for preserving harvested biological cells as described in clause 209, or any other clause, and further comprising the step of utilizing a single collection of biological cells for said step of using said biological cells for said predetermined use.
253. A method for preserving harvested biological cells as described in clause 212, or any other clause, wherein said step of using said biological cells for said predetermined use comprises the step of providing an improved post-warm cellular health.
254. A method for preserving harvested biological cells as described in clause 253, or any other clause, wherein said improved post-warm cellular health comprises greater than about 25% pregnancy rate artificial insemination of post-warmed bovine sperm cells.
255. A method for preserving harvested biological cells as described in clause 209, or any other clause, wherein said step of creating a uniform environment around substantially each of said biological cell comprises the step of encapsulating said biological cells.
256. A method for preserving harvested biological cells as described in clause 212, or any other clause, wherein said step of preserving said collection of said biological cells comprise the step of limiting oxygen exposure to said biological cells.
257. A method for preserving harvested biological cells as described in clause 209, or any other clause, wherein said step of creating said uniform environment around said biological cells comprises the step of creating a cage-like environment around each of said biological cells.
258. A method for preserving harvested biological cells as described in clause 257, or any other clause, wherein said step of creating a cage-like environment around each of said biological cells comprises the step of interacting compounds with a phospholipid head group of said biological cells.
259. A method for preserving harvested biological cells as described in clause 209, or any other clause, wherein said step of creating a uniform environment around said biological cells comprises the step of adding compounds to said collection of biological cells, said compounds selected from a group consisting of membrane lipids, glycolipids, cholesterol, free fatty acids, phosphoglycerides, sterols, sphingolipids, membrane proteins, salts, agarose, and any combination thereof.
260. A method for preserving harvested biological cells as described in clause 209, or any other clause, wherein said step of creating a uniform environment around said biological cells comprises the step of encapsulating said biological cells in a microenvironment.
261. A method for preserving harvested biological cells as described in clause 260, or any other clause, wherein said step of encapsulating said biological cells in a microenvironment comprise the step of adding liposomes or micelles to said collection of biological cells.
262. A method for preserving harvested biological cells as described in clause 260, or any other clause, wherein said step of encapsulating said biological cells in a microenvironment comprise the step of utilizing a microfluidic system.
263. A method for preserving harvested biological cells as described in clause 260, or any other clause, wherein said microenvironment comprises a component selected form a group consisting of antioxidant, plant lipid, egg yolk, and any combination thereof.
264. A method for preserving harvested biological cells as described in clause 260, or any other clause, and further comprising the step of surrounding said microenvironment with a media.
265. A method for preserving harvested biological cells as described in clause 264, or any other clause, wherein said media comprises agarose.
266. A method for preserving harvested biological cells as described in clause 260, or any other clause, wherein said microenvironment is processed selected from a group consisting of cooling said microenvironment to between about 0° C. to about 37° C., cooling said microenvironment to about 4° C., cooling said microenvironment to about 10° C., cooling said microenvironment to about 17° C., freezing said microenvironment, freezing said microenvironment to about −20° C., and freezing said microenvironment to about -196° C.
267. A method for preserving harvested biological cells as described in clause 260, or any other clause, and further comprising the step of releasing said microenvironment at 20° C. or up to 37° C.
268. A method for preserving harvested biological cells as described in clause 255, or any other clause, wherein said step of encapsulating said biological cells comprises a step selected from a group consisting of providing a micellular structure around said biological cells; providing a lipid layer around said biological cells, providing a lipid monolayer around said biological cells, and providing a lipid bilayer around said biological cells.
269. A method for preserving harvested biological cells as described in clause 257, or any other clause, wherein said cage-like environment comprises an encapsulation of said biological cells with a three-dimensional complex.
270. A method for preserving harvested biological cells as described in clause 260, or any other clause, wherein said microenvironment can be achieved by utilizing microfluidics to create said microenvironment.
271. A method for preserving harvested biological cells as described in clause 257, or any other clause, wherein said cage-like environment comprises compounds selected from a group consisting of lipids, salts, proteins, BSA protein, phosphatidyl serine, agarose, and any combination thereof.
272. A method for preserving harvested biological cells as described in clause 209, or any other clause, wherein said step of creating said uniform environment around said biological cells comprises the step of adding fatty acids to said collection of biological cells.
273. A method for preserving harvested biological cells as described in clause 272, or any other clause, wherein said step of adding fatty acids to said collection of biological cells comprises the step of adding from about 0.5% to about 10% v/v of fatty acids to said collection of biological cells.
274. A method for preserving harvested biological cells as described in clause 209, or any other clause, wherein said step of creating said uniform environment around said biological cells comprises the step of adding lipids containing about 40% linolenic acid (18:3), about 15% linoleic (18:2) and about 20% palmitic to said collection of biological cells.
275. A method for preserving harvested biological cells as described in clause 209, or any other clause, wherein said step of creating said uniform environment around said biological cells comprises the step of providing lipids and biological cells together encapsulated in a micellular or liposomal structure.
276. A method for preserving harvested biological cells as described in clause 209, or any other clause, wherein said step of creating said uniform environment around said biological cells comprises the step of adding a blend of lipids, free fatty acids, phospholipids, and cholesterol optimally beneficial to an individual cell type and a cell derivation.
277. A method for preserving harvested biological cells as described in clause 214, or any other clause, and further comprising the step of providing said holding media in a preservation kit for biological cells.
278. A method for preserving harvested biological cells as described in clause 277, or any other clause, wherein said preservation kit comprises at least two components selected from an antioxidant, a phospholipase inhibitor, and an antimicrobial agent.
279. A method for preserving harvested biological cells as described in clauses 215, 234, 240, 259, 271, 275, or any other clause, wherein said lipid is selected from a group consisting of lipids, free fatty acids, phospholipids, proteins, glycoproteins, and lipoproteins.
280. A method for maximizing viability of each cell in a collection of biological cells comprising the steps of:
   harvesting a collection of biological cells from an in vivo source;
   preserving said collection of said biological cells; and
   adding a phospholipase inhibitor to said collection of biological cells.
281. A biological cell transport preservation composition comprising:
   a collection of biological cells obtained from an in vivo source;
   a holding media comprising at least two components selected from an antioxidant, a phospholipase inhibitor, membrane stabilizing agent, and an antimicrobial agent, wherein said holding media is configured to be applied to said collection of biological cells before transporting said collection of biological cells, and wherein said holding media is applicable for an anticipated cell damage limiting regimen and a predetermined use of said collection of biological cells; and
   a hypothermic treatment preparation media to be applied to said collection of biological cells after said step of transporting said collection of biological cells.
282. A biological cell transport preservation composition as described in clause 281, or any other clause, wherein said collection of biological cells is selected from a group consisting of cells, tissues, sperm, equine sperm, bovine sperm, caprine sperm, ovine sperm, porcine sperm, fowl sperm, ovaries, oocytes, embryos, organs, stem cells, genetically modified cells, artificially derived cells, and any combination thereof.
283. A biological cell transport preservation composition as described in clause 281, or any other clause, wherein said in vivo source is selected from a group consisting of mammal, human, rodents, equine, bovine, caprine, ovine, porcine, fowl, fish, shell fish, reptile, nephropidae, poikilothermic, and aquatic vertebrates.
284. A biological cell transport preservation composition as described in clause 281, or any other clause, wherein said predetermined use is selected from a group consisting of insemination, implantation, culturing, research, diagnostic testing, replication, gamete preservation, genetic preservation, cryopreservation, reproduction, and any combination thereof.
285. A biological cell transport preservation composition as described in clause 281, or any other clause, wherein said holding media comprises at least one component selected from a group consisting of natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara Zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
286. A biological cell transport preservation composition as described in clause 285, or any other clause, wherein said plant extract comprises a plant extract derived from a source selected from a group consisting of sap, berries, seeds, leaves, flowers, stems, bark, and any combination thereof.
287. A biological cell transport preservation composition as described in clause 285, or any other clause, wherein said plant extract is selected from a group consisting of a crude plant extract, a single source plant extract, a combination of extracts from more than one source, alcohol extracts, juice components, sodium hydroxide extracts, aqueous extracts, hydroglycerine extracts, and any combination thereof.
288. A biological cell transport preservation composition as described in clause 281, or any other clause, wherein said holding media comprises an anti-microbial component selected from a group consisting of heptadecanoyl ethanolamide, triterpenes, steroid-like triterpenes, lipoglycopeptides, natural gums, natural resins, essential oils, tea tree oil, hyperenone A, hypercalin B, hyperphorin, phenolics, polyphenols, terpenes, flavonoids, alkaloids, propolis, spermidine, rutin, quercetin, coumarins, kaempferol, stigmasterol, campesterol, tocopherol, carotenoids, horseradish juice extract, tobramycin and any combination thereof.
289. A biological cell transport preservation composition as described in clause 285, or any other clause, wherein said phospholipase inhibitor comprises a phospholipase A2 inhibitor.
290. A biological cell transport preservation composition as described in clause 285, or any other clause, wherein said phospholipase inhibitor is selected from a group consisting of zinc, manganese, citric acid, and any combination thereof.
291. A biological cell transport preservation composition as described in clause 285, or any other clause, wherein said phospholipase inhibitor is selected from a group consisting of a plant extract, cucurmin, Gingko biloba extract, Centella asiatica extract, Hippophae extract, a chemical phospholipase inhibitor, pyrrolidone-based compounds, aristolochic acid, spermine neomycin sulfate, and any combination thereof.
292. A biological cell transport preservation composition as described in clause 285, or any other clause, wherein said microbial inhibitor is a plant derived component.
293. A biological cell transport preservation composition as described in clause 281, or any other clause, wherein said holding media comprises time released compounds in said holding media.
294. A biological cell transport preservation composition as described in clause 281, or any other clause, and further comprising additional holding media to be applied to said collection of biological cells during transportation of said collection of said biological cells.
295. A biological cell transport preservation composition as described in clause 285, or any other clause, wherein said cryoprotectant is selected from a group consisting of glycerol, glycine, dimethylsulfoxide, proline, modified betaines, glycinebetaine, dimethylsulphoniopropionate, cyclohexanediol, methyl formamide, dimethyl formamide, ethylene glycol, trehalose, concentrated complex sugars, tree sap, concentrated sugars, penetrating cryoprotectants, non-penetrating cryoprotectants, plant extracts, and any combination thereof.
296. A biological cell transport preservation composition as described in clause 281, or any other clause, and further comprising a cooler of said collection of said biological cells.
297. A biological cell transport preservation composition as described in clause 296, or any other clause, wherein said cooler is configured to cool said collection of biological cells to a temperature selected from a group consisting of between about 0° C. to about 37° C., about 4° C., about 10° C., and about 17° C.
298. A biological cell transport preservation composition as described in clause 296, or any other clause, wherein said cooler is configured to cool said collection of biological cells at a cooling rate from between about 0.01° C./min to about 1° C./min.
299. A biological cell transport preservation composition as described in clause 281, or any other clause, wherein said holding media comprises a pre-processing media.
300. A biological cell transport preservation composition as described in clause 281, or any other clause, wherein said holding media is configured to maintain an in vivo redox potential within said biological cells.
301. A biological cell transport preservation composition as described in clause 300, or any other clause, wherein said holding media configured to maintain an in vivo redox potential within said biological cells comprise a combination of lipid soluble and aqueous antioxidants in said holding media.
302. A biological cell transport preservation composition as described in clause 301, or any other clause, wherein said lipid soluble and aqueous antioxidants comprises a plant extract.
303. A biological cell transport preservation composition as described in clause 281, or any other clause, and further comprising a system selected from a group consisting of microfluidics, and flow cytometry.
304. A biological cell transport preservation composition as described in clause 281, or any other clause, and further comprising a uniform environment created around said biological cells, wherein said uniform environment is created by a system selected from a group consisting of microfluidics, encapsulation, creating liposomes, creating a micelle, creating a biological cage structure, and any combination thereof.
305. A biological cell transport preservation composition as described in clause 281, or any other clause, wherein said collection of said biological cells after transportation comprises a characteristic selected from a group consisting of reduced bacterial growth, increased bacteriostatic effect, and increased bactericidal effects.
306. A biological cell transport preservation composition as described in clause 281, or any other clause, wherein said hypothermic treatment preparation media is selected from a group consisting of antibiotics, natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara Zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
307. A biological cell transport preservation composition as described in clause 281, or any other clause, wherein said hypothermic treatment preparation media comprises less antibiotics, wherein said less antibiotics is selected from a group consisting of less than about 50 IU/ml penicillin, less than about 100 IU/ml penicillin, less than about50 μg/ml streptomycin, less than about 100 μg/ml streptomycin, less than about 500 ug/ml streptomycin, less than about 500 IU/ml penicillin, less than about 150 ug/ml lincomycin, and less than about 300 ug/ml spectinomycin.
308. A biological cell transport preservation composition as described in clause 281, or any other clause, wherein said hypothermic treatment preparation media comprises antibiotics that have been substituted at least in part with a plant extract.
309. A biological cell transport preservation composition as described in clause 308, or any other clause, wherein said step of substitution is selected from a group consisting of: about 10% of the antibiotic is substituted with a plant extract; about 20% of the antibiotic is substituted with a plant extract; about 30% of the antibiotic is substituted with a plant extract; about 40% of the antibiotic is substituted with a plant extract; about 50% of the antibiotic is substituted with a plant extract; about 60% of the antibiotic is substituted with a plant extract; about 70% of the antibiotic is substituted with a plant extract; about 80% of the antibiotic is substituted with a plant extract; about 90% of the antibiotic is substituted with a plant extract; and about 100% of the antibiotic is substituted with a plant extract.
310. A biological cell transport preservation composition as described in clause 281, or any other clause, wherein said hypothermic treatment preparation media comprises an antioxidant.
311. A biological cell transport preservation composition as described in clause 310, or any other clause, wherein said antioxidant is selected from a group consisting of allene oxide synthase, phenolics, flavonoids, ascorbic acid, tocopherols, carotenoids, tannins, butylated hydroxyanisole, butylated hydroxytoluene, tert-butylhydroxyquinone, propyl gallate, and compounds, plant derived or synthetic, sufficient to reduce or scavenge reactive oxygen species superoxide, hydroxyl, peroxyl, alkoxyl, nitric oxide, singlet oxygen, hydrogen peroxide, and any combination thereof.
312. A biological cell transport preservation composition as described in clause 281, or any other clause, wherein said anticipated cell damage limiting regimen comprises a reduction in cell damage, said cell damage caused from an aspect selected from a group consisting of biological contamination, chemical contamination, contamination caused by invasive species, chemical residues, detergents, disinfectant residues, solvent compounds, organic compounds, photo activation, photo modification, improper handling, bacteria, fungi, mycoplasma, virus, and any combination thereof.
313. A biological cell transport preservation composition as described in clause 306, or any other clause, wherein said osmotic agent comprise a plant extract.
314. A biological cell transport preservation composition as described in clause 281, or any other clause, wherein said hypothermic treatment is selected from a group consisting of cooling, cryopreservation, freeze-drying, lyophilization, and vitrification.
315. A biological cell transport preservation composition as described in clause 281, or any other clause, and further comprising an improved post-warm cellular health of said biological cells after a hypothermic treatment.
316. A biological cell transport preservation composition as described in clause 315, or any other clause, wherein said improved post-warm cellular health comprises greater than about 25% pregnancy rate artificial insemination of post-warmed bovine sperm cells.
317. A biological cell transport preservation composition as described in clause 281, or any other clause, and further comprising encapsulated biological cells.
318. A biological cell transport preservation composition as described in clause 281, or any other clause, wherein said biological cells comprises a limited oxygen exposure.
319. A biological cell transport preservation composition as described in clause 281, or any other clause, and further comprising a uniform environment around said biological cells.
320. A biological cell transport preservation composition as described in clause 319, or any other clause, wherein said uniform environment around said biological cells comprises a cage-like environment around each of said biological cells.
321. A biological cell transport preservation composition as described in clause 320, or any other clause, wherein said cage-like environment around each of said biological cells comprises compounds interacting with a phospholipid head group of said biological cells.
322. A biological cell transport preservation composition as described in clause 319, or any other clause, wherein said uniform environment comprises a compound selected from a group consisting of membrane lipids, glycolipids, cholesterol, free fatty acids, phosphoglycerides, sterols, sphingolipids, membrane proteins, salts, agarose, and any combination thereof.
323. A biological cell transport preservation composition as described in clause 319, or any other clause, wherein said uniform environment around said biological cells comprises encapsulated biological cells in a microenvironment.
324. A biological cell transport preservation composition as described in clause 323, or any other clause, wherein said encapsulated biological cells in said microenvironment comprises liposomes.
325. A biological cell transport preservation composition as described in clause 323, or any other clause, and further comprising a microfluidic system.
326. A biological cell transport preservation composition as described in clause 323, or any other clause, wherein said microenvironment comprises a component selected form a group consisting of antioxidant, plant lipid, egg yolk, and any combination thereof.
327. A biological cell transport preservation composition as described in clause 323, or any other clause, and further comprising a media surrounding said microenvironment.
328. A biological cell transport preservation composition as described in clause 327, or any other clause, wherein said media comprises agarose.
329. A biological cell transport preservation composition as described in clause 323, or any other clause, wherein said microenvironment is treated according to a treatment selected from a group consisting of cooled to about 4° C., frozen to about −20° C., and frozen to about -196° C.
330. A biological cell transport preservation composition as described in clause 323, or any other clause, and further comprising a microenvironment release at a temperature of about 20° C. or up to about 37° C.
331. A biological cell transport preservation composition as described in clause 317, or any other clause, wherein said encapsulated biological cells comprises a structure selected from a group consisting of a micellular structure; a lipid layer, a lipid monolayer, a lipid bilayer.
332. A biological cell transport preservation composition as described in clause 320, or any other clause, wherein said cage-like environment comprises an encapsulation of said biological cells with a three-dimensional complex.
333. A biological cell transport preservation composition as described in clause 323, or any other clause, wherein said microenvironment can be achieved by utilizing microfluidics to create said microenvironment.
334. A biological cell transport preservation composition as described in clause 323, or any other clause, wherein said cage-like environment comprises compounds selected from a group consisting of lipids, salts, proteins, BSA protein, phosphatidyl serine, agarose, and any combination thereof.
335. A biological cell transport preservation composition as described in clause 319, or any other clause, wherein said step of uniform environment comprises fatty acids.
336. A biological cell transport preservation composition as described in clause 335, or any other clause, wherein fatty acids comprises about 0.5% to about 10% v/v of fatty acids.
337. A biological cell transport preservation composition as described in clause 319, or any other clause, wherein said uniform environment comprises lipids containing about 40% linolenic acid (18:3), about 15% linoleic (18:2), and about 20% palmitic.
338. A biological cell transport preservation composition as described in clause 319, or any other clause, wherein said uniform environment comprises lipids and biological cells together encapsulated in a micellular or liposomal structure.
339. A biological cell transport preservation composition as described in clause 319, or any other clause, wherein said uniform environment comprises a blend of lipids, free fatty acids, phospholipids, and cholesterol optimally beneficial to an individual cell type and a cell derivation.
340. A biological cell transport preservation composition as described in clause 285, 301, 306, 322, 334, 338, 339, or any other clause, wherein said lipid is selected from a group consisting of lipids, free fatty acids, phospholipids, proteins, glycoproteins, and lipoproteins.
341. A biological cell transport preservation composition comprising:
   a collection of biological cells obtained from an in vivo source;
   a holding media to be applied to said collection of biological cells before transporting said collection of biological cells, said holding media applicable for an anticipated cell damage limiting regimen and a predetermined use of said collection of biological cells; and
   a hypothermic treatment preparation media to be applied to said collection of biological cells after said step of transporting said collection of biological cells.
342. A biological cell transport preservation composition as described in clause 341, or any other clause, wherein said collection of biological cells is selected from a group consisting of cells, tissues, sperm, equine sperm, bovine sperm, caprine sperm, ovine sperm, porcine sperm, fowl sperm, ovaries, oocytes, embryos, organs, stem cells, genetically modified cells, artificially derived cells, and any combination thereof.
343. A biological cell transport preservation composition as described in clause 341, or any other clause, wherein said in vivo source is selected from a group consisting of mammal, human, rodents, equine, bovine, caprine, ovine, porcine, fowl, fish, shell fish, reptile, nephropidae, poikilothermic, and aquatic vertebrates.
344. A biological cell transport preservation composition as described in clause 341, or any other clause, wherein said predetermined use is selected from a group consisting of insemination, implantation, culturing, research, diagnostic testing, replication, gamete preservation, genetic preservation, cryopreservation, reproduction, and any combination thereof.
345. A biological cell transport preservation composition as described in clause 341, or any other clause, wherein said holding media comprises at least one component selected from a group consisting of natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara Zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
346. A biological cell transport preservation composition as described in clause 345, or any other clause, wherein said plant extract comprises a plant extract derived from a source selected from a group consisting of sap, berries, seeds, leaves, flowers, stems, bark, and any combination thereof.
347. A biological cell transport preservation composition as described in clause 345, or any other clause, wherein said plant extract is selected from a group consisting of a crude plant extract, a single source plant extract, a combination of extracts from more than one source, alcohol extracts, juice components, sodium hydroxide extracts, aqueous extracts, hydroglycerine extracts, and any combination thereof.
348. A biological cell transport preservation composition as described in clause 341, or any other clause, wherein said holding media comprises an anti-microbial component selected from a group consisting of heptadecanoyl ethanolamide, triterpenes, steroid-like triterpenes, lipoglycopeptides, natural gums, natural resins, essential oils, tea tree oil, hyperenone A, hypercalin B, hyperphorin, phenolics, polyphenols, terpenes, flavonoids, alkaloids, propolis, spermidine, rutin, quercetin, coumarins, kaempferol, stigmasterol, campesterol, tocopherol, carotenoids, horseradish juice extract, tobramycin and any combination thereof.
349. A biological cell transport preservation composition as described in clause 341, or any other clause, wherein said holding media comprises at least two components selected from an antioxidant, a phospholipase inhibitor, membrane stabilizing agent, and an antimicrobial agent.
350. A biological cell transport preservation composition as described in clause 345, or any other clause, wherein said phospholipase inhibitor comprises a phospholipase A2 inhibitor.
351. A biological cell transport preservation composition as described in clause 345, or any other clause, wherein said phospholipase inhibitor is selected from a group consisting of zinc, manganese, citric acid, and any combination thereof.
352. A biological cell transport preservation composition as described in clause 345, or any other clause, wherein said phospholipase inhibitor is selected from a group consisting of a plant extract, cucurmin, Gingko biloba extract, Centella asiatica extract, Hippophae extract, a chemical phospholipase inhibitor, pyrrolidone-based compounds, aristolochic acid, spermine neomycin sulfate, and any combination thereof.
353. A biological cell transport preservation composition as described in clause 345, 349, or any other clause, wherein said microbial inhibitor is a plant derived component.
354. A biological cell transport preservation composition as described in clause 341, or any other clause, wherein said holding media comprises time released compounds in said holding media.
355. A biological cell transport preservation composition as described in clause 341, or any other clause, and further comprising additional holding media to be applied to said collection of biological cells during transportation of said collection of said biological cells.
356. A biological cell transport preservation composition as described in clause 345, or any other clause, wherein said cryoprotectant is selected from a group consisting of glycerol, glycine, dimethylsulfoxide, proline, modified betaines, glycinebetaine, dimethylsulphoniopropionate, cyclohexanediol, methyl formamide, dimethyl formamide, ethylene glycol, trehalose, concentrated complex sugars, tree sap, concentrated sugars, penetrating cryoprotectants, non-penetrating cryoprotectants, plant extracts, and any combination thereof.
357. A biological cell transport preservation composition as described in clause 341, or any other clause, and further comprising a cooler of said collection of said biological cells.
358. A biological cell transport preservation composition as described in clause 357, or any other clause, wherein said cooler is configured to cool said collection of biological cells to a temperature selected from a group consisting of between about 0° C. to about 37° C., about 4° C., about 10° C., and about 17° C.
359. A biological cell transport preservation composition as described in clause 357, or any other clause, wherein said cooler is configured to cool said collection of biological cells at a cooling rate from between about 0.01° C./min to about 1° C./min.
360. A biological cell transport preservation composition as described in clause 341, or any other clause, wherein said holding media comprises a pre-processing media.
361. A biological cell transport preservation composition as described in clause 341, or any other clause, wherein said holding media is configured to maintain an in vivo redox potential within said biological cells.
362. A biological cell transport preservation composition as described in clause 361, or any other clause, wherein said holding media configured to maintain an in vivo redox potential within said biological cells comprise a combination of lipid soluble and aqueous antioxidants in said holding media.
363. A biological cell transport preservation composition as described in clause 362, or any other clause, wherein said lipid soluble and aqueous antioxidants comprises a plant extract.
364. A biological cell transport preservation composition as described in clause 341, or any other clause, and further comprising a system selected from a group consisting of microfluidics, and flow cytometry.
365. A biological cell transport preservation composition as described in clause 341, or any other clause, and further comprising a uniform environment created around said biological cells, wherein said uniform environment is created by a system selected from a group consisting of microfluidics, encapsulation, creating liposomes, creating a micelle, creating a biological cage structure, and any combination thereof.
366. A biological cell transport preservation composition as described in clause 341, or any other clause, wherein said collection of said biological cells after transportation comprises a characteristic selected from a group consisting of reduced bacterial growth, increased bacteriostatic effect, and increased bactericidal effects.
367. A biological cell transport preservation composition as described in clause 341, or any other clause, wherein said hypothermic treatment preparation media is selected from a group consisting of antibiotics, natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
368. A biological cell transport preservation composition as described in clause 34, or any other clause,1 wherein said hypothermic treatment preparation media comprises less antibiotics, wherein said less antibiotics is selected from a group consisting of less than about 50 IU/ml penicillin, less than about 100 IU/ml penicillin, less than about50 μg/ml streptomycin, less than about 100 μg/ml streptomycin, less than about 500 ug/ml streptomycin, less than about 500 IU/ml penicillin, less than about 150 ug/ml lincomycin, and less than about 300 ug/ml spectinomycin.
369. A biological cell transport preservation composition as described in clause 341, or any other clause, wherein said hypothermic treatment preparation media comprises antibiotics that have been substituted at least in part with a plant extract.
370. A biological cell transport preservation composition as described in clause 369 wherein said step of substitution is selected from a group consisting of: about 10% of the antibiotic is substituted with a plant extract; about 20% of the antibiotic is substituted with a plant extract; about 30% of the antibiotic is substituted with a plant extract; about 40% of the antibiotic is substituted with a plant extract; about 50% of the antibiotic is substituted with a plant extract; about 60% of the antibiotic is substituted with a plant extract; about 70% of the antibiotic is substituted with a plant extract; about 80% of the antibiotic is substituted with a plant extract; about 90% of the antibiotic is substituted with a plant extract; and about 100% of the antibiotic is substituted with a plant extract.
371. A biological cell transport preservation composition as described in clause 341, or any other clause, wherein said hypothermic treatment preparation media comprises an antioxidant.
372. A biological cell transport preservation composition as described in clause 371, or any other clause, wherein said antioxidant comprises is selected from a group consisting of allene oxide synthase, phenolics, flavonoids, ascorbic acid, tocopherols, carotenoids, tannins, butylated hydroxyanisole, butylated hydroxytoluene, tert-butylhydroxyquinone, propyl gallate, and compounds, plant derived or synthetic, sufficient to reduce or scavenge reactive oxygen species superoxide, hydroxyl, peroxyl, alkoxyl, nitric oxide, singlet oxygen, hydrogen peroxide, and any combination thereof.
373. A biological cell transport preservation composition as described in clause 341, or any other clause, wherein said anticipated cell damage limiting regimen comprises a reduction in cell damage, said cell damage caused from an aspect selected from a group consisting of biological contamination, chemical contamination, contamination caused by invasive species, chemical residues, detergents, disinfectant residues, solvent compounds, organic compounds, photo activation, photo modification, improper handling, bacteria, fungi, mycoplasma, virus, and any combination thereof.
374. A biological cell transport preservation composition as described in clause 367, or any other clause, wherein said osmotic agent comprise a plant extract.
375. A biological cell transport preservation composition as described in clause 341, or any other clause, wherein said hypothermic treatment is selected from a group consisting of cooling, cryopreservation, freeze-drying, lyophilization, and vitrification.
376. A biological cell transport preservation composition as described in clause 341, or any other clause, and further comprising an improved post-warm cellular health of said biological cells after a hypothermic treatment.
377. A biological cell transport preservation composition as described in clause 376, or any other clause, wherein said improved post-warm cellular health comprises greater than about 25% pregnancy rate artificial insemination of post-warmed bovine sperm cells.
378. A biological cell transport preservation composition as described in clause 341, or any other clause, and further comprising encapsulated biological cells.
379. A biological cell transport preservation composition as described in clause 341, or any other clause, wherein said biological cells comprises a limited oxygen exposure.
380. A biological cell transport preservation composition as described in clause 341, or any other clause, and further comprising a uniform environment around said biological cells.
381. A biological cell transport preservation composition as described in clause 380, or any other clause, wherein said uniform environment around said biological cells comprises a cage-like environment around each of said biological cells.
382. A biological cell transport preservation composition as described in clause 381, or any other clause, wherein said cage-like environment around each of said biological cells comprises compounds interacting with a phospholipid head group of said biological cells.
383. A biological cell transport preservation composition as described in clause 380, or any other clause, wherein said uniform environment comprises a compound selected from a group consisting of membrane lipids, glycolipids, cholesterol, free fatty acids, phosphoglycerides, sterols, sphingolipids, membrane proteins, salts, agarose, and any combination thereof.
384. A biological cell transport preservation composition as described in clause 380, or any other clause, wherein said uniform environment around said biological cells comprises encapsulated biological cells in a microenvironment.
385. A biological cell transport preservation composition as described in clause 384, or any other clause, wherein said encapsulated biological cells in said microenvironment comprises liposomes.
386. A biological cell transport preservation composition as described in clause 384, or any other clause, and further comprising a microfluidic system.
387. A biological cell transport preservation composition as described in clause 384, or any other clause, wherein said microenvironment comprises a component selected form a group consisting of antioxidant, plant lipid, egg yolk, and any combination thereof.
388. A biological cell transport preservation composition as described in clause 384, or any other clause, and further comprising a media surrounding said microenvironment.
389. A biological cell transport preservation composition as described in clause 338, or any other clause, wherein said media comprises agarose.
390. A biological cell transport preservation composition as described in clause 384, or any other clause, wherein said microenvironment is treated according to a treatment selected from a group consisting of cooled to about 4° C., frozen to about −20° C., and frozen to about −196° C.
391. A biological cell transport preservation composition as described in clause 384, or any other clause, and further comprising a microenvironment release at a temperature of about 20° C. or up to about 37° C.
392. A biological cell transport preservation composition as described in clause 378, or any other clause, wherein said encapsulated biological cells comprises a structure selected from a group consisting of a micellular structure; a lipid layer, a lipid monolayer, a lipid bilayer.
393. A biological cell transport preservation composition as described in clause 381, or any other clause, wherein said cage-like environment comprises an encapsulation of said biological cells with a three-dimensional complex.
394. A biological cell transport preservation composition as described in clause 384, or any other clause, wherein said microenvironment can be achieved by utilizing microfluidics to create said microenvironment.
395. A biological cell transport preservation composition as described in clause 381, or any other clause, wherein said cage-like environment comprises compounds selected from a group consisting of lipids, salts, proteins, BSA protein, phosphatidyl serine, agarose, and any combination thereof.
396. A biological cell transport preservation composition as described in clause 380, or any other clause, wherein said step of uniform environment comprises fatty acids.
397. A biological cell transport preservation composition as described in clause 396, or any other clause, wherein fatty acids comprises about 0.5% to about 10% v/v of fatty acids.
398. A biological cell transport preservation composition as described in clause 380, or any other clause, wherein said uniform environment comprises lipids containing about 40% linolenic acid (18:3), about 15% linoleic (18:2), and about 20% palmitic.
399. A biological cell transport preservation composition as described in clause 380, or any other clause, wherein said uniform environment comprises lipids and biological cells together encapsulated in a micellular or liposomal structure.
400. A biological cell transport preservation composition as described in clause 380, or any other clause, wherein said uniform environment comprises a blend of lipids, free fatty acids, phospholipids, and cholesterol optimally beneficial to an individual cell type and a cell derivation.
401. A biological cell transport preservation composition as described in clause 345, 362, 367, 383, 395, 399, 400, or any other clause, wherein said lipid is selected from a group consisting of lipids, free fatty acids, phospholipids, proteins, glycoproteins, and lipoproteins.
402. A biological cell preservation composition comprising:
   a collection of biological cells obtained from an in vivo source; and
   a uniform environment established around each biological cell of a collection of said biological cells; and
   a phospholipase inhibitor.
403. A biological cell preservation composition as described in clause 402, or any other clause, wherein said collection of biological cells is selected from a group consisting of cells, tissues, sperm, equine sperm, bovine sperm, caprine sperm, ovine sperm, porcine sperm, fowl sperm, ovaries, oocytes, embryos, organs, stem cells, genetically modified cells, artificially derived cells, and any combination thereof.
404. A biological cell preservation composition as described in clause 402, or any other clause, wherein said in vivo source is selected from a group consisting of mammal, human, rodents, equine, bovine, caprine, ovine, porcine, fowl, fish, shell fish, reptile, nephropidae, poikilothermic, and aquatic vertebrates.
405. A biological cell preservation composition as described in clause 402, or any other clause, and further comprising a holding media configured to be applied to said collection of biological cells, wherein said holding media is configured to be applicable for an anticipated cell damage limiting regimen and a predetermined use of said collection of biological cells
406. A biological cell preservation composition as described in clause 405, or any other clause, wherein said predetermined use is selected from a group consisting of insemination, implantation, culturing, research, diagnostic testing, replication, gamete preservation, genetic preservation, cryopreservation, reproduction, and any combination thereof.
407. A biological cell preservation composition as described in clause 405, or any other clause, wherein said holding media comprises at least one component selected from a group consisting of natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
408. A biological cell preservation composition as described in clause 407, or any other clause, wherein said plant extract comprises a plant extract derived from a source selected from a group consisting of sap, berries, seeds, leaves, flowers, stems, bark, and any combination thereof.
409. A biological cell preservation composition as described in clause 407, or any other clause, wherein said plant extract is selected from a group consisting of a crude plant extract, a single source plant extract, a combination of extracts from more than one source, alcohol extracts, juice components, sodium hydroxide extracts, aqueous extracts, hydroglycerine extracts, and any combination thereof.
410. A biological cell preservation composition as described in clause 405, or any other clause, wherein said holding media comprises an anti-microbial component selected from a group consisting of heptadecanoyl ethanolamide, triterpenes, steroid-like triterpenes, lipoglycopeptides, natural gums, natural resins, essential oils, tea tree oil, hyperenone A, hypercalin B, hyperphorin, phenolics, polyphenols, terpenes, flavonoids, alkaloids, propolis, spermidine, rutin, quercetin, coumarins, kaempferol, stigmasterol, campesterol, tocopherol, carotenoids, horseradish juice extract, tobramycin and any combination thereof.
411. A biological cell preservation composition as described in clause 405, or any other clause, wherein said holding media comprises at least two components selected from an antioxidant, a phospholipase inhibitor, membrane stabilizing agent, and an antimicrobial agent.
412. A biological cell preservation composition as described in clause 402, or any other clause, wherein said phospholipase inhibitor comprises a phospholipase A2 inhibitor.
413. A biological cell preservation composition as described in clause 402, or any other clause, wherein said phospholipase inhibitor is selected from a group consisting of zinc, manganese, citric acid, and any combination thereof.
414. A biological cell preservation composition as described in clause 402, or any other clause, wherein said phospholipase inhibitor is selected from a group consisting of a plant extract, cucurmin, Gingko biloba extract, Centella asiatica extract, Hippophae extract, a chemical phospholipase inhibitor, pyrrolidone-based compounds, aristolochic acid, spermine neomycin sulfate, and any combination thereof.
415. A biological cell preservation composition as described in clause 402, or any other clause, wherein said microbial inhibitor is a plant derived component.
416. A biological cell preservation composition as described in clause 405, or any other clause, wherein said holding media comprises time released compounds in said holding media.
417. A biological cell preservation composition as described in clause 405, or any other clause, and further comprising additional holding media to be applied to said collection of biological cells during transportation of said collection of said biological cells.
418. A biological cell preservation composition as described in clause 407, or any other clause, wherein said cryoprotectant is selected from a group consisting of glycerol, glycine, dimethylsulfoxide, proline, modified betaines, glycinebetaine, dimethylsulphoniopropionate, cyclohexanediol, methyl formamide, dimethyl formamide, ethylene glycol, trehalose, concentrated complex sugars, tree sap, concentrated sugars, penetrating cryoprotectants, non-penetrating cryoprotectants, plant extracts, and any combination thereof.
419. A biological cell preservation composition as described in clause 402, or any other clause, and further comprising a cooler of said collection of said biological cells.
420. A biological cell preservation composition as described in clause 419, or any other clause, wherein said cooler is configured to cool said collection of biological cells to a temperature selected from a group consisting of between about 0° C. to about 37° C., about 4° C., about 10° C., and about 17° C.
421. A biological cell preservation composition as described in clause 419, or any other clause, wherein said cooler is configured to cool said collection of biological cells at a cooling rate from between about 0.01° C./min to about 1° C./min.
422. A biological cell preservation composition as described in clause 405, or any other clause, wherein said holding media comprises a pre-processing media.
423. A biological cell preservation composition as described in clause 405, or any other clause, wherein said holding media is configured to maintain an in vivo redox potential within said biological cells.
424. A biological cell preservation composition as described in clause 423, or any other clause, wherein said holding media configured to maintain an in vivo redox potential within said biological cells comprise a combination of lipid soluble and aqueous antioxidants in said holding media.
425. A biological cell preservation composition as described in clause 424, or any other clause, wherein said lipid soluble and aqueous antioxidants comprises a plant extract.
426. A biological cell preservation composition as described in clause 402, or any other clause, and further comprising a system selected from a group consisting of microfluidics, and flow cytometry.
427. A biological cell preservation composition as described in clause 402, or any other clause, wherein said uniform environment is created by a system selected from a group consisting of microfluidics, encapsulation, creating liposomes, creating a micelle, creating a biological cage structure, and any combination thereof.
428. A biological cell preservation composition as described in clause 402, or any other clause, wherein said collection of said biological cells after transportation comprises a characteristic selected from a group consisting of reduced bacterial growth, increased bacteriostatic effect, and increased bactericidal effects.
429. A biological cell preservation composition as described in clause 402, or any other clause, and further comprising a hypothermic treatment preparation media selected from a group consisting of antibiotics, natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
430. A biological cell preservation composition as described in clause 402, or any other clause, and further comprising a hypothermic treatment preparation media comprising less antibiotics, wherein said less antibiotics is selected from a group consisting of less than about 50 IU/ml penicillin, less than about 100 IU/ml penicillin, less than about50 μg/ml streptomycin, less than about 100 μg/ml streptomycin, less than about 500 ug/ml streptomycin, less than about 500 IU/ml penicillin, less than about 150 ug/ml lincomycin, and less than about 300 ug/ml spectinomycin.
431. A biological cell preservation composition as described in clause 402, or any other clause, and further comprising a hypothermic treatment preparation media comprising antibiotics that have been substituted at least in part with a plant extract.
432. A biological cell preservation composition as described in clause 431, or any other clause, wherein said step of substitution is selected from a group consisting of: about 10% of the antibiotic is substituted with a plant extract; about 20% of the antibiotic is substituted with a plant extract; about 30% of the antibiotic is substituted with a plant extract; about 40% of the antibiotic is substituted with a plant extract; about 50% of the antibiotic is substituted with a plant extract; about 60% of the antibiotic is substituted with a plant extract; about 70% of the antibiotic is substituted with a plant extract; about 80% of the antibiotic is substituted with a plant extract; about 90% of the antibiotic is substituted with a plant extract; and about 100% of the antibiotic is substituted with a plant extract.
433. A biological cell preservation composition as described in clause 402, or any other clause, and further comprising a hypothermic treatment preparation media comprising an antioxidant.
434. A biological cell preservation composition as described in clause 433, or any other clause, wherein said antioxidant is selected from a group consisting of allene oxide synthase, phenolics, flavonoids, ascorbic acid, tocopherols, carotenoids, tannins, butylated hydroxyanisole, butylated hydroxytoluene, tert-butylhydroxyquinone, propyl gallate, and compounds, plant derived or synthetic, sufficient to reduce or scavenge reactive oxygen species superoxide, hydroxyl, peroxyl, alkoxyl, nitric oxide, singlet oxygen, hydrogen peroxide, and any combination thereof.
435. A biological cell preservation composition as described in clause 405, or any other clause, wherein said anticipated cell damage limiting regimen comprises a reduction in cell damage, said cell damage caused from an aspect selected from a group consisting of biological contamination, chemical contamination, contamination caused by invasive species, chemical residues, detergents, disinfectant residues, solvent compounds, organic compounds, photo activation, photo modification, improper handling, bacteria, fungi, mycoplasma, virus, and any combination thereof.
436. A biological cell preservation composition as described in clause 429, or any other clause, wherein said osmotic agent comprise a plant extract.
437. A biological cell preservation composition as described in clause 402, or any other clause, and further comprising a hypothermic treatment is selected from a group consisting of cooling, cryopreservation, freeze-drying, lyophilization, and vitrification.
438. A biological cell preservation composition as described in clause 402, or any other clause, and further comprising an improved post-warm cellular health of said biological cells after a hypothermic treatment.
439. A biological cell preservation composition as described in clause 438, or any other clause, wherein said improved post-warm cellular health comprises greater than about 25% pregnancy rate artificial insemination of post-warmed bovine sperm cells.
440. A biological cell preservation composition as described in clause 402, or any other clause, wherein said uniform environment comprises encapsulated biological cells.
441. A biological cell preservation composition as described in clause 402, or any other clause, wherein said biological cells comprises a limited oxygen exposure.
442. A biological cell preservation composition as described in clause 402, or any other clause, wherein said uniform environment around said biological cells comprises a cage-like environment around each of said biological cells.
443. A biological cell preservation composition as described in clause 442, or any other clause, wherein said cage-like environment around each of said biological cells comprises compounds interacting with a phospholipid head group of said biological cells.
444. A biological cell preservation composition as described in clause 402, or any other clause, wherein said uniform environment comprises a compound selected from a group consisting of membrane lipids, glycolipids, cholesterol, free fatty acids, phosphoglycerides, sterols, sphingolipids, membrane proteins, salts, agarose, and any combination thereof.
445. A biological cell preservation composition as described in clause 402, or any other clause, wherein said uniform environment around said biological cells comprises encapsulated biological cells in a microenvironment.
446. A biological cell preservation composition as described in clause 445, or any other clause, wherein said encapsulated biological cells in said microenvironment comprises liposomes.
447. A biological cell preservation composition as described in clause 445, or any other clause, and further comprising a microfluidic system.
448. A biological cell preservation composition as described in clause 445, or any other clause, wherein said microenvironment comprises a component selected form a group consisting of antioxidant, plant lipid, egg yolk, and any combination thereof.
449. A biological cell preservation composition as described in clause 445, or any other clause, and further comprising a media surrounding said microenvironment.
450. A biological cell preservation composition as described in clause 449, or any other clause, wherein said media comprises agarose.
451. A biological cell preservation composition as described in clause 445, or any other clause, wherein said microenvironment is treated according to a treatment selected from a group consisting of cooled to about 4° C., frozen to about −20° C., and frozen to about −196° C.
452. A biological cell preservation composition as described in clause 445, or any other clause, and further comprising a microenvironment release at a temperature of about 20° C. or up to about 37° C.
453. A biological cell preservation composition as described in clause 440, or any other clause, wherein said encapsulated biological cells comprises a structure selected from a group consisting of a micellular structure; a lipid layer, a lipid monolayer, a lipid bilayer.
454. A biological cell preservation composition as described in clause 442, or any other clause, wherein said cage-like environment comprises an encapsulation of said biological cells with a three-dimensional complex.
455. A biological cell preservation composition as described in clause 445, or any other clause, wherein said microenvironment can be achieved by utilizing microfluidics to create said microenvironment.
456. A biological cell preservation composition as described in clause 442, or any other clause, wherein said cage-like environment comprises compounds selected from a group consisting of lipids, salts, proteins, BSA protein, phosphatidyl serine, agarose, and any combination thereof.
457. A biological cell preservation composition as described in clause 402, or any other clause, wherein said step of uniform environment comprises fatty acids.
458. A biological cell preservation composition as described in clause 457, or any other clause, wherein fatty acids comprises about 0.5% to about 10% v/v of fatty acids.
459. A biological cell preservation composition as described in clause 402, or any other clause, wherein said uniform environment comprises lipids containing about 40% linolenic acid (18:3), about 15% linoleic (18:2), and about 20% palmitic.
460. A biological cell preservation composition as described in clause 402, or any other clause, wherein said uniform environment comprises lipids and biological cells together encapsulated in a micellular or liposomal structure.
461. A biological cell preservation composition as described in clause 402, or any other clause, wherein said uniform environment comprises a blend of lipids, free fatty acids, phospholipids, and cholesterol optimally beneficial to an individual cell type and a cell derivation.
462. A biological cell preservation composition as described in clause 407, 424, 429, 444, 456, 460, 461, or any other clause, wherein said lipid is selected from a group consisting of lipids, free fatty acids, phospholipids, proteins, glycoproteins, and lipoproteins.
463. A biological cell preservation composition comprising:
   a collection of biological cells obtained from an in vivo source;
   a holding media to be applied to said collection of biological cells, said holding media configured to establish a uniform environment around each biological cell; and
   a hypothermic treatment preparation media to be applied to said collection of biological cells after said step of transporting said collection of biological cells.
464. A biological cell preservation composition as described in clause 463, or any other clause, wherein said collection of biological cells is selected from a group consisting of cells, tissues, sperm, equine sperm, bovine sperm, caprine sperm, ovine sperm, porcine sperm, fowl sperm, ovaries, oocytes, embryos, organs, stem cells, genetically modified cells, artificially derived cells, and any combination thereof.
465. A biological cell preservation composition as described in clause 463, or any other clause, wherein said in vivo source is selected from a group consisting of mammal, human, rodents, equine, bovine, caprine, ovine, porcine, fowl, fish, shell fish, reptile, nephropidae, poikilothermic, and aquatic vertebrates.
466. A biological cell preservation composition as described in clause 463, or any other clause, wherein said holding media is configured to be applicable for an anticipated cell damage limiting regimen and a predetermined use of said collection of biological cells
467. A biological cell preservation composition as described in clause 466, or any other clause, wherein said predetermined use is selected from a group consisting of insemination, implantation, culturing, research, diagnostic testing, replication, gamete preservation, genetic preservation, cryopreservation, reproduction, and any combination thereof.
468. A biological cell preservation composition as described in clause 463, or any other clause, wherein said holding media comprises at least one component selected from a group consisting of natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemical s, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Tetrapleura Hippophae rhamnoides, tetraptera extract, silibinin, phosphofructokinase, carnosine, lignans, fagaronine, ellagitannins, eschscholtzidine, saponin, and any combination thereof.
469. A biological cell preservation composition as described in clause 468, or any other clause, wherein said plant extract comprises a plant extract derived from a source selected from a group consisting of sap, berries, seeds, leaves, flowers, stems, bark, and any combination thereof.
470. A biological cell preservation composition as described in clause 468, or any other clause, wherein said plant extract is selected from a group consisting of a crude plant extract, a single source plant extract, a combination of extracts from more than one source, alcohol extracts, juice components, sodium hydroxide extracts, aqueous extracts, hydroglycerine extracts, and any combination thereof.
471. A biological cell preservation composition as described in clause 463, or any other clause, wherein said holding media comprises an anti-microbial component selected from a group consisting of heptadecanoyl ethanolamide, triterpenes, steroid-like triterpenes, lipoglycopeptides, natural gums, natural resins, essential oils, tea tree oil, hyperenone A, hypercalin B, hyperphorin, phenolics, polyphenols, terpenes, flavonoids, alkaloids, propolis, spermidine, rutin, quercetin, coumarins, kaempferol, stigmasterol, campesterol, tocopherol, carotenoids, horseradish juice extract, tobramycin and any combination thereof.
472. A biological cell preservation composition as described in clause 463, or any other clause, wherein said holding media comprises at least two components selected from an antioxidant, a phospholipase inhibitor, membrane stabilizing agent, and an antimicrobial agent.
473. A biological cell preservation composition as described in clause 468, or any other clause, wherein said phospholipase inhibitor comprises a phospholipase A2 inhibitor.
474. A biological cell preservation composition as described in clause 468, or any other clause, wherein said phospholipase inhibitor is selected from a group consisting of zinc, manganese, citric acid, and any combination thereof.
475. A biological cell preservation composition as described in clause 468, or any other clause, wherein said phospholipase inhibitor is selected from a group consisting of a plant extract, cucurmin, Gingko biloba extract, Centella asiatica extract, Hippophae extract, a chemical phospholipase inhibitor, pyrrolidone-based compounds, aristolochic acid, spermine neomycin sulfate, and any combination thereof.
476. A biological cell preservation composition as described in clause 468, 472, or any other clause, wherein said microbial inhibitor is a plant derived component.
477. A biological cell preservation composition as described in clause 463, or any other clause, wherein said holding media comprises time released compounds in said holding media.
478. A biological cell preservation composition as described in clause 463, or any other clause, and further comprising additional holding media to be applied to said collection of biological cells during transportation of said collection of said biological cells.
479. A biological cell preservation composition as described in clause 468, or any other clause, wherein said cryoprotectant is selected from a group consisting of glycerol, glycine, dimethylsulfoxide, proline, modified betaines, glycinebetaine, dimethylsulphoniopropionate, cyclohexanediol, methyl formamide, dimethyl formamide, ethylene glycol, trehalose, concentrated complex sugars, tree sap, concentrated sugars, penetrating cryoprotectants, non-penetrating cryoprotectants, plant extracts, and any combination thereof.
480. A biological cell preservation composition as described in clause 463, or any other clause, and further comprising a cooler of said collection of said biological cells.
481. A biological cell preservation composition as described in clause 480, or any other clause, wherein said cooler is configured to cool said collection of biological cells to a temperature selected from a group consisting of between about 0° C. to about 37° C., about 4° C., about 10° C., and about 17° C.
482. A biological cell preservation composition as described in clause 480, or any other clause, wherein said cooler is configured to cool said collection of biological cells at a cooling rate from between about 0.01° C./min to about 1° C./min.
483. A biological cell preservation composition as described in clause 463, or any other clause, wherein said holding media comprises a pre-processing media.
484. A biological cell preservation composition as described in clause 463, or any other clause, wherein said holding media is configured to maintain an in vivo redox potential within said biological cells.
485. A biological cell preservation composition as described in clause 484, or any other clause, wherein said holding media configured to maintain an in vivo redox potential within said biological cells comprise a combination of lipid soluble and aqueous antioxidants in said holding media.
486. A biological cell preservation composition as described in clause 485, or any other clause, wherein said lipid soluble and aqueous antioxidants comprises a plant extract.
487. A biological cell preservation composition as described in clause 463, or any other clause, and further comprising a system selected from a group consisting of microfluidics, and flow cytometry.
488. A biological cell preservation composition as described in clause 463, or any other clause, wherein said uniform environment is created by a system selected from a group consisting of microfluidics, encapsulation, creating liposomes, creating a micelle, creating a biological cage structure, and any combination thereof.
489. A biological cell preservation composition as described in clause 463, or any other clause, wherein said collection of said biological cells after transportation comprises a characteristic selected from a group consisting of reduced bacterial growth, increased bacteriostatic effect, and increased bactericidal effects.
490. A biological cell preservation composition as described in clause 463, or any other clause, and further comprising a hypothermic treatment preparation media
491. A biological cell preservation composition as described in clause 463, or any other clause, wherein said hypothermic treatment preparation media selected from a group consisting of antibiotics, natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, Tetrapleura tetraptera extract, silibinin, phosphofructokinase, carnosine, lignans, fagaronine, ellagitannins, eschscholtzidine, saponin, and any combination thereof.
492. A biological cell preservation composition as described in clause 490, or any other clause, wherein said hypothermic treatment preparation media comprises less antibiotics, wherein said less antibiotics is selected from a group consisting of less than about 50 IU/ml penicillin, less than about 100 IU/ml penicillin, less than about50 μg/ml streptomycin, less than about 100 μg/ml streptomycin, less than about 500 ug/ml streptomycin, less than about 500 IU/ml penicillin, less than about 150 ug/ml lincomycin, and less than about 300 ug/ml spectinomycin.
493. A biological cell preservation composition as described in clause 490, or any other clause, wherein said hypothermic treatment preparation media comprises antibiotics that have been substituted at least in part with a plant extract.
494. A biological cell preservation composition as described in clause 493, or any other clause, wherein said step of substitution is selected from a group consisting of: about 10% of the antibiotic is substituted with a plant extract; about 20% of the antibiotic is substituted with a plant extract; about 30% of the antibiotic is substituted with a plant extract; about 40% of the antibiotic is substituted with a plant extract; about 50% of the antibiotic is substituted with a plant extract; about 60% of the antibiotic is substituted with a plant extract; about 70% of the antibiotic is substituted with a plant extract; about 80% of the antibiotic is substituted with a plant extract; about 90% of the antibiotic is substituted with a plant extract; and about 100% of the antibiotic is substituted with a plant extract.
495. A biological cell preservation composition as described in clause 490, or any other clause, wherein said hypothermic treatment preparation media comprises an antioxidant.
496. A biological cell preservation composition as described in clause 495, or any other clause, wherein said antioxidant comprises is selected from a group consisting of allene oxide synthase, phenolics, flavonoids, ascorbic acid, tocopherols, carotenoids, tannins, butylated hydroxyanisole, butylated hydroxytoluene, tert-butylhydroxyquinone, propyl gallate, and compounds, plant derived or synthetic, sufficient to reduce or scavenge reactive oxygen species superoxide, hydroxyl, peroxyl, alkoxyl, nitric oxide, singlet oxygen, hydrogen peroxide, and any combination thereof.
497. A biological cell preservation composition as described in clause 466, or any other clause, wherein said anticipated cell damage limiting regimen comprises a reduction in cell damage, said cell damage caused from an aspect selected from a group consisting of biological contamination, chemical contamination, contamination caused by invasive species, chemical residues, detergents, disinfectant residues, solvent compounds, organic compounds, photo activation, photo modification, improper handling, bacteria, fungi, mycoplasma, virus, and any combination thereof.
498. A biological cell preservation composition as described in clause 491, or any other clause, wherein said osmotic agent comprise a plant extract.
499. A biological cell preservation composition as described in clause 490, or any other clause, wherein said hypothermic treatment is selected from a group consisting of cooling, cryopreservation, freeze-drying, lyophilization, and vitrification.
500. A biological cell preservation composition as described in clause 463, or any other clause, and further comprising an improved post-warm cellular health of said biological cells after a hypothermic treatment.
501. A biological cell preservation composition as described in clause 500, or any other clause, wherein said improved post-warm cellular health comprises greater than about 25% pregnancy rate artificial insemination of post-warmed bovine sperm cells.
502. A biological cell preservation composition as described in clause 463, or any other clause, and further comprising encapsulated biological cells.
503. A biological cell preservation composition as described in clause 463, or any other clause, wherein said biological cells comprises a limited oxygen exposure.
504. A biological cell preservation composition as described in clause 463, or any other clause, wherein said uniform environment around said biological cells comprises a cage-like environment around each of said biological cells.
505. A biological cell preservation composition as described in clause 504, or any other clause, wherein said cage-like environment around each of said biological cells comprises compounds interacting with a phospholipid head group of said biological cells.
506. A biological cell preservation composition as described in clause 463, or any other clause, wherein said uniform environment comprises a compound selected from a group consisting of membrane lipids, glycolipids, cholesterol, free fatty acids, phosphoglycerides, sterols, sphingolipids, membrane proteins, salts, agarose, and any combination thereof.
507. A biological cell preservation composition as described in clause 463, or any other clause, wherein said uniform environment around said biological cells comprises encapsulated biological cells in a microenvironment.
508. A biological cell preservation composition as described in clause 507, or any other clause, wherein said encapsulated biological cells in said microenvironment comprises liposomes.
509. A biological cell preservation composition as described in clause 507, or any other clause, and further comprising a microfluidic system.
510. A biological cell preservation composition as described in clause 507, or any other clause, wherein said microenvironment comprises a component selected form a group consisting of antioxidant, plant lipid, egg yolk, and any combination thereof.
511. A biological cell preservation composition as described in clause 507, or any other clause, and further comprising a media surrounding said microenvironment.
512. A biological cell preservation composition as described in clause 511, or any other clause, wherein said media comprises agarose.
513. A biological cell preservation composition as described in clause 507, or any other clause, wherein said microenvironment is treated according to a treatment selected from a group consisting of cooled to about 4° C., frozen to about −20° C., and frozen to about −196° C.
514. A biological cell preservation composition as described in clause 507, or any other clause, and further comprising a microenvironment release at a temperature of about 20° C. or up to about 37° C.
515. A biological cell preservation composition as described in clause 502, or any other clause, wherein said encapsulated biological cells comprises a structure selected from a group consisting of a micellular structure; a lipid layer, a lipid monolayer, a lipid bilayer.
516. A biological cell preservation composition as described in clause 504, or any other clause, wherein said cage-like environment comprises an encapsulation of said biological cells with a three-dimensional complex.
517. A biological cell preservation composition as described in clause 507, or any other clause, wherein said microenvironment can be achieved by utilizing microfluidics to create said microenvironment.
518. A biological cell preservation composition as described in clause 504, or any other clause, wherein said cage-like environment comprises compounds selected from a group consisting of lipids, salts, proteins, BSA protein, phosphatidyl serine, agarose, and any combination thereof.
519. A biological cell preservation composition as described in clause 463, or any other clause, wherein said step of uniform environment comprises fatty acids.
520. A biological cell preservation composition as described in clause 519, or any other clause, wherein fatty acids comprises about 0.5% to about 10% v/v of fatty acids.
521. A biological cell preservation composition as described in clause 463, or any other clause, wherein said uniform environment comprises lipids containing about 40% linolenic acid (18:3), about 15% linoleic (18:2), and about 20% palmitic.
522. A biological cell preservation composition as described in clause 463, or any other clause, wherein said uniform environment comprises lipids and biological cells together encapsulated in a micellular or liposomal structure.
523. A biological cell preservation composition as described in clause 463, or any other clause, wherein said uniform environment comprises a blend of lipids, free fatty acids, phospholipids, and cholesterol optimally beneficial to an individual cell type and a cell derivation.
524. A biological cell preservation composition as described in clause 485, 491, 506, 518, 522, 523, or any other clause, wherein said lipid is selected from a group consisting of lipids, free fatty acids, phospholipids, proteins, glycoproteins, and lipoproteins.
530. A biological cell preservation composition as described in clause 472, or any other clause, wherein said antimicrobial agent comprises a bacteriostatic compound or a bacteriocidal compound.
531. A system substantially as herein described with reference to any one or more of the Figures and Description.

As can be easily understood from the foregoing, the basic concepts of the present invention may be embodied in a variety of ways. It involves both cell preserving techniques as well as devices to accomplish the appropriate cell preservation. In this application, the cell preserving techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “collection” should be understood to encompass disclosure of the act of “collecting”—whether explicitly discussed or not—and, conversely, were there effectively disclosure of the act of “collecting,” such a disclosure should be understood to encompass disclosure of a “collection” and even a “means for collecting.” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the below list of references or other information statement filed with the application are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

### I. US PATENTS

| | | | |
|---|---|---|---|
| 6,864,046 | B1 | 2005 Mar. 8 | Prien et al. |
| 8,685,563 | B1 | 2014 Apr. 1 | Lin |
| 6,982,172 | B2 | 2006 Jan. 3 | Yang et al. |
| 5,358,931 | | 1994 Oct. 25 | Rubinsky et al. |
| 6,238,920 | B1 | 2001 May 29 | Nagai et al. |
| 6,395,305 | B1 | 2002 May 28 | Buhr et al. |

### II. US PATENT PUBLICATIONS

| | | | |
|---|---|---|---|
| 2010/0196872 | A1 | 2010 Aug. 5 | Loskutoff et al. |
| 2017/0367324 | A1 | 2017 Dec. 28 | Lenz et al. |

### III. NON PATENT LITERATURE

Kono, Hajime, Dipti Karmarkar, Yoichiro Iwakura, and Kenneth L.
Rock, 2010, “Identification of the Cellular Sensor That Stimulates the
Inflammatory Response to Sterile Cell Death,” The Journal of
Immunology, 184: 4470-78.
Qin Zhang, Mustafa Raoof, Chen Yu, Sumi Yuka, Tolga Sursal, Junger
Wolfgang, Karim Brohi, Kiyoshi Itagaki, and Carl J. Hauser, 2010,
“Circulating mitochondrial DAMPs cause inflammatory responses to
injury,” Nature, 464: 104-07.
Rubinsky, Boris, 2003, “Principles of Low Temperature Cell
Preservation,” Heart Failure Reviews, 8: 277-84.
Ganley, O. H., Graessle, O. E., Robinson, H. J., et al., “Anti-inflammatory
activity of compounds obtained from egg yolk, peanut oil, and soybean
lecithin,” Journal of Laboratory and Clinical Medicine 51, 709-714
(1958).
Baoru Yang, Riina M. Karlsson, Pentti H. Oksman, and Kallio, H. P.,
“Phytosterols in Sea Buckthorn (Hippophaë rhamnoides L.) Berries:
Identification and Effects of Different Origins and Harvesting Times,”
(2001), doi: 10.1021/JF010813M, 5620-5629.
Mokoka, T. A. et al., “Antimicrobial activity and cytotoxicity of
triterpenes isolated from leaves of Maytenus undata (Celastraceae),”
BMC Complement. Altern. Med. 13, 111 (2013), 9 pages.
Michel, T., Destandau, E., Le Floch, G., Lucchesi, M. E. & Elfakir, C.,
“Antimicrobial, antioxidant and phytochemical investigations of sea
buckthorn (Hippophaë rhamnoides L.) leaf, stem, root and seed,”
Food Chem. 131, 754-760 (2012).
Johnson, L. A., et al. (2000) ″Storage of boar semen.″ Animal
Reproduction Science 62(1): 143-172.
United States Provisional Patent Application No. 62/594,394, filed
December 4, 2017. First Named Inventor: Herickhoff.
United States Provisional Patent Application No. 62/589,422, filed
November 21, 2017. First Named Inventor: Herickhoff.
Iritani, A. and Y. Nishikawa (1963), ″Studies on the egg-coagulating
enzyme in goat semen: IV. On the position of yolk constituents attacked
by the coagulating enzyme,″ Jpn J Anim Reprod 8(4): 113-117.
Purdy, P. (2006), ″A review on goat sperm cryopreservation,″ Small
Ruminant Research 63(3):215-225.
Roy, A. (1957), “Egg yolk-coagulating enzyme in the semen and
Cowper's gland of the goat,″ Nature 179 (4554): 318.
Rasband, W.S., ImageJ, U. S. National Institutes of Health, Bethesda,
Maryland, USA, https://imagej.nih.gov/ij/, 1997-2018.

Thus, the applicant(s) should be understood to have support to claim and make a statement of invention to at least: i) each of the cell preservation devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such processes, methods, systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 20 or any other claim” or the like, it could be re-drafted as dependent on claim 1, claim 15, or even claim 25 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

1. A method of protecting in vitro biological cells with synergistic continuity comprising the steps of:
harvesting a collection of biological cells from an in vivo source;
preserving said collection of said biological cells based on an anticipated cell damage limiting regimen and a predetermined use;
providing a holding media applicable for said anticipated cell damage limiting regimen and said predetermined use, wherein said holding media comprises at least two components selected from an antioxidant, a phospholipase inhibitor, membrane stabilizing agent, and an antimicrobial agent;
adding said holding media to said collection of said biological cells;
transporting said collection of said biological cells in said holding media based on said anticipated cell damage limiting regimen and said predetermined use;
receiving said collection of said biological cells after said step of transporting said collection of said biological cells in said holding media;
preparing said biological cells to be hypothermically treated;
hypothermically treating said biological cells;
warming said biological cells; and
using said biological cells for said predetermined use.

2. A method of protecting in vitro biological cells as described in claim 1 wherein said collection of biological cells is chosen from: cells, tissues, sperm, equine sperm, bovine sperm, caprine sperm, ovine sperm, porcine sperm, fowl sperm, ovaries, oocytes, embryos, organs, stem cells, genetically modified cells, artificially derived cells, and any combination thereof.

3. A method of protecting in vitro biological cells as described in claim 1 wherein said in vivo source is chosen from: mammal, human, rodents, equine, bovine, caprine, ovine, porcine, fowl, fish, shell fish, reptile, nephropidae, poikilothermic, and aquatic vertebrates.

4. A method of protecting in vitro biological cells as described in claim 1 wherein said predetermined use is chosen from: insemination, implantation, culturing, research, diagnostic testing, replication, gamete preservation, genetic preservation, cryopreservation, reproduction, and any combination thereof.

5. A method of protecting in vitro biological cells as described in claim 1 wherein said holding media comprises at least one additional component chosen from: natural ingredients, non-animal derived components, microbial inhibitor, bacteriostatic compound, bactericidal compound, a compound that inhibits bacterial replication, antibacterial component, phospholipase inhibitor, phospholipase A2 inhibitor, anti-inflammatory compound, immune suppressant compound, antiprotease compound, membrane stabilizing compound, cryoprotectant, osmotic agent, buffer, extender, antioxidant, ice nucleator, chemically defined media, vitamin E, vitamin C, trehalose, cholesterol, lecithin, phytochemicals, carbohydrates, phenolics, polyphenol, organic acids, lipid, sugar, salt, protein, compound molecules, phytochemicals, secondary metabolites of plants, plant extract, sea buckthorn extract, Fagara zanthoxyloides extract, Olax subscorpioides extract, Hippophae rhamnoides, or Tetrapleura tetraptera extract, silibinin, phosphofructokinase, carnosine, lignans, fagaronine, ellagitannins, eschscholtzidine, saponin, and any combination thereof.

6. (canceled)

7. A method of protecting in vitro biological cells as described in claim 5 wherein said plant extract is chosen from: a crude plant extract, a single source plant extract, a combination of extracts from more than one source, alcohol extracts, juice components, sodium hydroxide extracts, aqueous extracts, hydroglycerine extracts, and any combination thereof.

8. A method of protecting in vitro biological cells as described in claim 1 wherein said holding media comprises an anti-microbial component chosen from: heptadecanoyl ethanolamide, triterpenes, steroid-like triterpenes, lipoglycopeptides, natural gums, natural resins, essential oils, tea tree oil, hyperenone A, hypercalin B, hyperphorin, phenolics, polyphenols, terpenes, flavonoids, alkaloids, propolis, spermidine, rutin, quercetin, coumarins, kaempferol, stigmasterol, campesterol, tocopherol, carotenoids, horseradish juice extract, tobramycin and any combination thereof.

9. (canceled)

10. A method of protecting in vitro biological cells as described in claim 1 wherein said phospholipase inhibitor is chosen from: zinc, manganese, citric acid, and any combination thereof.

11.-12. (canceled)

13. A method of protecting in vitro biological cells as described in claim 1 wherein said step of providing said holding media applicable for said anticipated cell damage limiting regimen and said predetermined use comprises the step of providing time released compounds in said holding media.

14. (canceled)

15. A method of protecting in vitro biological cells as described in claim 5 wherein said cryoprotectant is chosen from: glycerol, glycine, dimethylsulfoxide, proline, modified betaines, glycinebetaine, dimethylsulphoniopropionate, cyclohexanediol, methyl formamide, dimethyl formamide, ethylene glycol, trehalose, concentrated complex sugars, tree sap, concentrated sugars, penetrating cryoprotectants, non-penetrating cryoprotectants, plant extracts, and any combination thereof.

16. (canceled)

17. A method of protecting in vitro biological cells as described in claim 1 wherein said step of transporting said collection of said biological cells in said holding media comprises the step of cooling said collection of said biological cells in said holding media.

18. (canceled)

19. A method of protecting in vitro biological cells as described in claim 17 wherein said step of cooling said collection of biological cells comprises the step of cooling said collection of biological cells at a cooling rate from between about 0.01° C./min to about 1° C./min.

20-25. (canceled)

26. A method of protecting in vitro biological cells as described in claim 1 wherein said step of receiving said collection of said biological cells after said step of transporting said biological cells in said holding media comprises the step of providing shipped biological cells with a characteristic selected from a group consisting of reduced bacterial growth, increased bacteriostatic effect, and increased bactericidal effects.

27-29. (canceled)

30. A method of protecting in vitro biological cells as described in claim 1 wherein said step of preparing said biological cells to be hypothermically treated comprises the step of adding antibiotics to said shipped biological cells and substituting at least part of said antibiotics with a plant extract.

31-34. (canceled)

35. A method of protecting in vitro biological cells as described in claim 1 wherein said anticipated cell damage limiting regimen comprises a reduction in cell damage, said cell damage caused from an aspect selected from a group consisting of biological contamination, chemical contamination, contamination caused by invasive species, chemical residues, detergents, disinfectant residues, solvent compounds, organic compounds, photo activation, photo modification, improper handling, bacteria, fungi, mycoplasma, virus, and any combination thereof.

36-37. (canceled)

38. A method of protecting in vitro biological cells as described in claim 1 wherein said step of preparing said biological cells to be hypothermically treated and said step of hypothermically treating said biological cells comprises a hypothermic treatment selected from a group consisting of cooling, cryopreservation, freeze-drying, lyophilization, and vitrification.

39-44. (canceled)

45. A method of protecting in vitro biological cells as described in claim 1 wherein said step of preserving said collection of said biological cells comprises the step of creating a uniform environment around said biological cells.

46. A method of protecting in vitro biological cells as described in claim 45 wherein said step of creating said uniform environment around said biological cells comprises the step of creating a cage-like environment around each of said biological cells.

47. A method of protecting in vitro biological cells as described in claim 46 wherein said step of creating a cage-like environment around each of said biological cells comprises the step of interacting compounds with a phospholipid head group of said biological cells.

48-62. (canceled)

63. A method of protecting in vitro biological cells as described in claim 45 wherein said step of creating said uniform environment around said biological cells comprises the step of adding lipids containing about 40% linolenic acid (18:3), about 15% linoleic (18:2) and about 20% palmitic to said collection of biological cells.

64-77. (canceled)
